(19) United States
(12) Patent Application Publication
(10) Pub. No.: US 2024/0408370 A1
(43) Pub. Date: Dec. 12, 2024

(54) System, Method, and Computer Program Product for Vascular Access Management

(71) Applicant: BECTON, DICKINSON AND COMPANY, Franklin Lakes, NJ (US)

(72) Inventors: Ramkumar Saptharishi, Bangalore (IN); Aswin Anand, Malappuram (IN); Shayan Ghosh, Bangalore (IN); Jack Balji, Mahwah, NJ (US); Vinay Parthan, Montville, NJ (US); Erik Kurt Witt, Oakland, NJ (US); Mark Jesser, Wyckoff, NJ (US); Ashley Rachel Rothenberg, Morris Plains, NJ (US); Yashar Seyed Vahedein, Mountain View, CA (US); Philip Ponce de Leon, Brooklyn, NY (US); Maarten Brand, Ridgewood, NJ (US); Mark Andrew Nelson, Harrison, NJ (US)

(21) Appl. No.: 18/694,777

(22) PCT Filed: Sep. 26, 2022
(86) PCT No.: PCT/US2022/044693

§ 371 Date: Mar. 22, 2024

Related U.S. Application Data
(60) Provisional application No. 63/248,818, filed on Sep. 27, 2021.

(51) Int. Cl. A61M 39/02; A61M 5/14; A61M 5/168; G16H 50/30

(52) U.S. Cl. CPC A61M 39/0247; A61M 5/16804; G16H 50/30; A61M 2005/1403; A61M 2039/0205; A61M 2039/0258; A61M 2205/3306; A61M 2205/332; A61M 2205/3334; A61M 2205/50

(57) ABSTRACT

A system, a method, and a computer program product for vascular access management: obtain vascular access management (VAM) data associated with a vascular access treatment associated with a patient; determine an insight associated with the vascular access treatment associated with the patient; and provide the insight associated with the vascular access treatment.

### CROSS-REFERENCE TO RELATED APPLICATIONS

This application is the United States national phase of International Application No. PCT/US2022/044693 filed Sep. 26, 2022, and claims priority to U.S. Provisional Application Ser. No. 63/248,818, entitled “System, Method, and Computer Program Product for Vascular Access Management”, filed Sep. 27, 2021, the entire disclosures of which are hereby incorporated by reference in their entireties.

### BACKGROUND

Vascular access treatment includes drug Infusion into a human body that is often achieved through catheters that are either inserted into a peripheral vein (PIVC) or central vein (PICC/CVC). A catheter may be connected to a fluid source, such as a pump, and/or the like, via a needleless connector.

A clinician providing a vascular access treatment may be affected by various factors, such as a high cognitive load due to a large variety of products used for vascular access treatment and a learning curve required for their usage, a large variety of patient profiles, a busy schedule, a lack of experience and/or expertise, and/or the like. These factors can lead to non-standardized practices and/or not using the correct device at the correct time during a vascular access treatment, which may lead to patients being exposed to various complications, such as phlebitis, occlusion, infiltration, Catheter Related Blood Stream Infection (CRBSI), Central Line-associated Bloodstream Infection (CLABSI), and/or the like. These complications may result in additional complications and/or increased costs through additional treatment, an incorrect choice of medical devices and insertion locations for vascular access treatment, an incorrect impression about the medical devices, additional pressure on more experienced staff, patient dissatisfaction impact hospital reputation, and/or the like.

Hospitals and homecare patient environments (e.g., in which nurses, caregivers, patient maintenance activities, etc. can be monitored) have adopted protocols that are aimed at ensuring proper catheter maintenance. However, multiple studies have shown poor adherence to these existing protocols, leading to sub-optimal patient outcomes. Moreover, these existing protocols do not support vascular access management due to different causes that create complications requiring different solutions.

### SUMMARY

Accordingly, provided are improved systems, devices, products, apparatus, and/or methods for vascular access management that obtain vascular access management (VAM) data associated with a vascular access treatment associated with a patient; determine an insight associated with the vascular access treatment associated with the patient; and provide the insight associated with the vascular access treatment.

In accordance with an embodiment of the present invention, a system includes at least one processor programmed and/or configured to obtain vascular access management (VAM) data associated with a vascular access treatment associated with a patient, determine an insight associated with the vascular access treatment associated with the patient, and provide the insight associated with the vascular access treatment.

In accordance with an embodiment of the present invention, the at least one processor is programmed and/or configured to determine the insight associated with the vascular access treatment associated with the patient by determining, based on the VAM data, an initial risk prediction for the vascular access treatment associated with the patient, wherein the initial risk prediction includes a probability that the patient experiences at least one complication in response to the vascular access treatment. The system also determines, based on the VAM data and the initial risk prediction, a recommendation associated with the vascular access treatment associated with the patient, wherein the recommendation includes at least one of a recommended process and a recommend product to be used for the vascular access treatment. The system also determines, based on the VAM data and the recommendation, an updated risk prediction for the vascular access treatment associated with the patient. The system also determines, based on the VAM data, the initial risk predication, the recommendation, and the updated risk prediction, a cost prediction associated with the vascular access treatment associated with the patient, wherein the cost prediction includes a predicted savings in terms of a reduced cost of complication from adoption of the at least one of the recommended process and the recommend product.

In accordance with an embodiment of the present invention, the at least one processor provides the insight by providing, to a user device, at least one of the following: the initial risk predication, the recommendation, and the updated risk prediction, the cost prediction, or any combination thereof.

In accordance with an embodiment of the present invention, the at least one processor provides the insight by automatically controlling, based on the insight, at least one medical device to adjust a flow of a fluid to the patient during the vascular access treatment.

In accordance with an embodiment of the present invention, the at least one processor is programmed and/or configured to obtain the VAM data by collecting, from a plurality of different data sources, source data, associating the source data with at least one clinical protocol, and aggregating the source data associated with the at least one clinical protocol as the VAM data associated with the vascular access treatment associated with the patient.

In accordance with an embodiment of the present invention, the VAM data includes one or more of the following parameters: a patient identifier; a hospital identifier; a patient name; a patient gender; a patient age, a co-morbidity associated with a patient; a medication associated with a patient, a symptom associated with a patient; a reason for admission associated with a patient; an infusion type associated with a patient; an admission date associated with a patient; a readmission indicator associated with a patient; a discharge date associated with a patient; a length of stay associated with a patient; a number of lines used associated with a patient; a type of accessories used associated with a patient; a date of use associated with a medical device; an average dwell time associated with a medical device, an average number of stick attempts associated with a patient, a complication associated with a patient; a department of a hospital; a user or nurse identifier; a user or nurse experience indicator; a question associated with a vascular access treatment; a question identifier associated with a question; an answer associated with a question; a time stamp associated with a usage of a medical device; a device identifier associated with a medical device, a type of a medical device, a device signal associated with a medical device; a number of occlusion cases in a period of time, a number of CRBSI and/or CLABSI cases in a time period; a predicted vascular signal (e.g., CRBSI, phlebitis, etc.); or any combination thereof.

In accordance with an embodiment of the present invention, the system of further includes a plurality of local systems, wherein each local system includes a central computing system, a sensor system including at least one sensor, and a user device, and a management system configured as a central unit or command center for remotely monitoring line maintenance activities at each local system of the plurality of local systems.

In accordance with an embodiment of the present invention, the system further includes one or more image capture devices configured to capture, over a period of time, a plurality of images of an environment surrounding the one or more image capture devices. The at least one processor is further programmed and/or configured to determine, based on the plurality of images, a plurality of locations of a plurality of medical devices within the environment over the period of time and a plurality of types of the plurality of medical devices. The processor is further programmed and/or configured to determine, based on the plurality of locations of the plurality of medical devices within the environment over the period of time and the plurality of types of the plurality of medical devices, at least a portion of the VAM data associated with the vascular access treatment associated with the patient.

In accordance with an embodiment of the present invention, further including a plurality of identifier elements associated with a plurality of medical devices, wherein the plurality of identifier elements encapsulates a plurality of identifiers associated a plurality of types of the plurality of medical devices. The system further includes one or more image capture devices configured to capture, over a period of time, a plurality of images of an environment surrounding the one or more image capture devices. The at least one processor is further programmed and/or configured to determine, based on the plurality of images, the plurality of identifier elements within the environment over the period of time, and determine, based on the plurality of identifier elements determined in the plurality of images, the plurality of types of the plurality of medical devices and a plurality of locations of the plurality of medical devices within the environment over the period of time. The processor is further programmed and/or configured to determine, based on the plurality of types of the plurality of medical devices and the plurality of locations of the plurality of medical devices within the environment over the period of time, at least a portion of the VAM data associated with the vascular access treatment associated with the patient.

In accordance with an embodiment of the present invention, the plurality of identifier elements includes at least one identifier element including at least one of the following types of identifier elements: a colored pattern, a reflective pattern, a fluorescent pattern, a predetermined shape and color, a LED pattern, a barcode, or any combination thereof.

In accordance with an embodiment of the present invention, the system further includes one or more image capture devices configured to capture, over a period of time, a plurality of images of an environment surrounding the one or more image capture devices. The at least one processor is further programmed and/or configured to determine, based on the plurality of images, a plurality of locations of a plurality of medical devices within the environment over the period of time and a plurality of types of the plurality of medical devices, and determine, based on the plurality of locations of the plurality of medical devices within the environment over the period of time, a plurality of distances between the plurality of medical devices over the period of time. The at least one processor is programmed and/or configured to determine, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices, and to determine, based on the at least one determined event, at least a portion of the VAM data associated with the vascular access treatment associated with the patient.

In accordance with an embodiment of the present invention, the system further includes a first identifier element associated with a medical device, wherein the first identifier element encapsulates a first identifier associated with the medical device, a second identifier element associated with a glove of a caregiver, wherein the second identifier element encapsulates a second identifier associated with the glove of the caregiver, one or more image capture devices configured to capture, over a period of time, a plurality of images of an environment surrounding the one or more image capture devices. The at least one processor is further programmed and/or configured to determine, based on the plurality of images, the first identifier element associated with the medical device and the second identifier element associated with the glove of a caregiver, determine, based on the first identifier element in the plurality of images, the medical device and a location of the medical device within the environment over the period of time, determine, based on the second identifier element in the plurality of images, the glove of the caregiver and a location of the glove of the caregiver within the environment over the period of time, determine, based on the location of the medical device within the environment over the period of time and the location of the glove of the caregiver within the environment over the period of time, at least one event associated with the medical device, and determine, based on the at least one determined event, at least a portion of the VAM data associated with the vascular access treatment associated with the patient.

In accordance with an embodiment of the present invention, the system further includes one or more image capture devices configured to capture, over a period of time, a plurality of images of an environment surrounding the one or more image capture devices, wherein the at least one processor is further programmed and/or configured to determine, based on the plurality of images, a location of a plunger of a syringe relative to a barrel of the syringe in the environment over the period of time, determine, based on the location of the plunger of the syringe relative to the barrel of the syringe over the period of time, at least one fluid delivery from the syringe, and determine, based on the at least one determined fluid delivery, at least a portion of the VAM data associated with the vascular access treatment associated with the patient.

In accordance with an embodiment of the present invention, the system further includes a package containing a medical device, one or more image capture devices configured to capture, over a period of time, a plurality of images of an environment surrounding the one or more image capture devices, and wherein the at least one processor is further programmed and/or configured to determine, based on the plurality of images, a state of the package over the period of time, determine, based on the state of the package over the period of time, whether the medical device is removed from the package, and determine, based on a determination that the medical device is removed from the package, at least a portion of the VAM data associated with the vascular access treatment associated with the patient.

In accordance with an embodiment of the present invention, further including a needleless connector including a fluid flow path, and a force sensor connected to the needleless connector. The at least one processor is further programmed and/or configured to receive, from the force sensor, a force signal, and determine, based on the force signal, at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof. The at least one processor is further programmed and/or configured to determine, based on the at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof, at least a portion of the VAM data associated with the vascular access treatment associated with the patient.

In accordance with an embodiment of the present invention, wherein the force sensor is positioned between an outer surface of an inner wall of the needleless connector defining the fluid flow path of the needleless connector and an inner surface of an outer wall of the needleless connector surrounding the inner wall of the needleless connector.

In accordance with an embodiment of the present invention, a first end of the needleless connector includes a septum including a surface facing in a first direction, wherein at least one of the force sensors is configured to detect a force in a second direction perpendicular to the surface of the septum facing in the first direction, and wherein the at least one processor are further programmed and/or configured to determine, based on the force signal indicating periodic forces in the second direction perpendicular to the surface of the septum facing in the first direction, the flushing event, wherein the flushing event includes a pulsatile flushing event.

In accordance with an embodiment of the present invention, the system further includes a needleless connector including a fluid flow path, a force sensor configured to measure a force signal, and a visual indicator, wherein the at least one processor is further programmed and/or configured to receive, from the force sensor, a force signal. The at least one processor further programmed and/or configured to determine, based on the force signal, at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof, and control the visual indicator to provide a visual indication associated with the at least one of: the scrubbing event in which the needleless connector is scrubbed with the disinfectant, the flushing event in which the needleless connector is flushed with the solution, the connection event in which the needleless connector is connected to the medical device, the disconnection event in which the needleless connector is disconnected from the medical device, or any combination thereof.

In accordance with an embodiment of the present invention, further including a needleless connector including a fluid flow path, an acoustic sensor connected to the needleless connector, wherein the at least one processor is further programmed and/or configured to receive, from the acoustic sensor, a signal including a sound signature, determine, based on the signal, an event associated with the needleless connector, and determine, based on the determined event associated with the needleless connector, at least a portion of the VAM data associated with the vascular access treatment associated with the patient.

In accordance with an embodiment of the present invention, the system further includes a needleless connector including a fluid flow path and a septum, an optical sensor connected to the needleless connector, wherein the optical sensor is configured to detect a movement of the septum, wherein the at least one processor is further programmed and/or configured to receive, from the optical sensor, a signal associated with the movement of the septum, determine, based on the signal, an event associated with the needleless connector, and determine, based on the determined event associated with the needleless connector, at least a portion of the VAM data associated with the vascular access treatment associated with the patient.

In accordance with an embodiment of the present invention, a method includes obtaining, with at least one processor, vascular access management (VAM) data associated with a vascular access treatment associated with a patient, determining, with the at least one processor, an insight associated with the vascular access treatment associated with the patient, and providing, with the at least one processor, the insight associated with the vascular access treatment.

In accordance with an embodiment of the present invention, the method includes that the insight associated with the vascular access treatment associated with the patient is determined by determining, based on the VAM data, an initial risk prediction for the vascular access treatment associated with the patient, wherein the initial risk prediction includes a probability that the patient experiences at least one complication in response to the vascular access treatment, determining, based on the VAM data and the initial risk prediction, a recommendation associated with the vascular access treatment associated with the patient, wherein the recommendation includes at least one of a recommended process and a recommend product to be used for the vascular access treatment, determining, based on the VAM data and the recommendation, an updated risk prediction for the vascular access treatment associated with the patient, and determining, based on the VAM data, the initial risk predication, the recommendation, and the updated risk prediction, a cost prediction associated with the vascular access treatment associated with the patient, wherein the cost prediction includes a predicted savings in terms of a reduced cost of complication from adoption of the at least one of the recommended process and the recommend product.

In accordance with an embodiment of the present invention, the at least one processor provides the insight by providing, to a user device, at least one of the following: the initial risk predication, the recommendation, and the updated risk prediction, the cost prediction, or any combination thereof.

In accordance with an embodiment of the present invention, the at least one processor provides the insight by automatically controlling, based on the insight, at least one medical device to adjust a flow of a fluid to the patient during the vascular access treatment.

In accordance with an embodiment of the present invention, the at least one processor obtains the VAM data by collecting, from a plurality of different data sources, source data, associating the source data with at least one clinical protocol, and aggregating the source data associated with the at least one clinical protocol as the VAM data associated with the vascular access treatment associated with the patient.

In accordance with an embodiment of the present invention, the VAM data includes one or more of the following parameters: a patient identifier; a hospital identifier; a patient name; a patient gender; a patient age, a co-morbidity associated with a patient; a medication associated with a patient, a symptom associated with a patient; a reason for admission associated with a patient; an infusion type associated with a patient; an admission date associated with a patient; a readmission indicator associated with a patient; a discharge date associated with a patient; a length of stay associated with a patient; a number of lines used associated with a patient; a type of accessories used associated with a patient; a date of use associated with a medical device; an average dwell time associated with a medical device, an average number of stick attempts associated with a patient, a complication associated with a patient; a department of a hospital; a user or nurse identifier; a user or nurse experience indicator; a question associated with a vascular access treatment; a question identifier associated with a question; an answer associated with a question; a time stamp associated with a usage of a medical device; a device identifier associated with a medical device, a type of a medical device, a device signal associated with a medical device; a number of occlusion cases in a period of time, a number of CRBSI and/or CLABSI cases in a time period; a predicted vascular signal (e.g., CRBSI, phlebitis, etc.); or any combination thereof.

In accordance with an embodiment of the present invention, the method further includes remotely monitoring, with a management system configured as a central unit or command center, line maintenance activities at a plurality of local systems, wherein each local system includes a central computing system, a sensor system including at least one sensor, and a user device.

In accordance with an embodiment of the present invention, the method further includes capturing, with one or more image capture devices, over a period of time, a plurality of images of an environment surrounding the one or more image capture devices. The method further includes determining, with the at least one processor, based on the plurality of images, a plurality of locations of a plurality of medical devices within the environment over the period of time and a plurality of types of the plurality of medical devices, and determining, with the at least one processor, based on the plurality of locations of the plurality of medical devices within the environment over the period of time and the plurality of types of the plurality of medical devices, at least a portion of the VAM data associated with the vascular access treatment associated with the patient.

In accordance with an embodiment of the present invention, the method further includes a plurality of identifier elements associated with a plurality of medical devices, wherein the plurality of identifier elements encapsulates a plurality of identifiers associated a plurality of types of the plurality of medical devices. The method also includes capturing, with one or more image capture devices, over a period of time, a plurality of images of an environment surrounding the one or more image capture devices, determining, with the at least one processor, based on the plurality of images, a plurality of identifier elements within the environment over the period of time, wherein the plurality of identifier elements is associated with a plurality of medical devices, and wherein the plurality of identifier elements encapsulates a plurality of identifiers associated a plurality of types of the plurality of medical devices, and determining, with the at least one processor, based on the plurality of identifier elements determined in the plurality of images, the plurality of types of the plurality of medical devices and a plurality of locations of the plurality of medical devices within the environment over the period of time.

In accordance with an embodiment of the present invention, the plurality of identifier elements includes at least one identifier element including at least one of the following types of identifier elements: a colored pattern, a reflective pattern, a fluorescent pattern, a predetermined shape and color, a LED pattern, a barcode, or any combination thereof.

In accordance with an embodiment of the present invention, the method further includes capturing, with one or more image capture devices, over a period of time, a plurality of images of an environment surrounding the one or more image capture devices, determining, with the at least one processor, based on the plurality of images, a plurality of locations of a plurality of medical devices within the environment over the period of time and a plurality of types of the plurality of medical devices, determining, with the at least one processor, based on the plurality of locations of the plurality of medical devices within the environment over the period of time, a plurality of distances between the plurality of medical devices over the period of time, determining, with the at least one processor, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices, and determining, with the at least one processor, based on the at least one determined event, at least a portion of the VAM data associated with the vascular access treatment associated with the patient.

In accordance with an embodiment of the present invention, the method further includes capturing, with one or more image capture devices, over a period of time, a plurality of images of an environment surrounding the one or more image capture devices, determining, with the at least one processor, based on the plurality of images, a first identifier element associated with a medical device and a second identifier element associated with a glove of a caregiver, wherein the first identifier element encapsulates a first identifier associated with the medical device, and wherein the second identifier element encapsulates a second identifier associated with the glove of the caregiver, determining, with the at least one processor, based on the first identifier element in the plurality of images, the medical device and a location of the medical device within the environment over the period of time, determining, with the at least one processor, based on the second identifier element in the plurality of images, the glove of the caregiver and a location of the glove of the caregiver within the environment over the period of time, determining, with the at least one processor, based on the location of the medical device within the environment over the period of time and the location of the glove of the caregiver within the environment over the period of time, at least one event associated with the medical device, and determining, with the at least one processor, based on the at least one determined event, at least a portion of the VAM data associated with the vascular access treatment associated with the patient.

In accordance with an embodiment of the present invention, the method further includes capturing, with one or more image capture devices, over a period of time, a plurality of images of an environment surrounding the one or more image capture devices, determining, with the at least one processor, based on the plurality of images, a location of a plunger of a syringe relative to a barrel of the syringe in the environment over the period of time, determining, with the at least one processor, based on the location of the plunger of the syringe relative to the barrel of the syringe over the period of time, at least one fluid delivery from the syringe, and determining, with the at least one processor, based on the at least one determined fluid delivery, at least a portion of the VAM data associated with the vascular access treatment associated with the patient.

In accordance with an embodiment of the present invention, the method further includes a package containing a medical device, one or more image capture devices configured to capturing, with one or more image capture devices, over a period of time, a plurality of images of an environment surrounding the one or more image capture devices, determining, with the at least one processor, based on the plurality of images, a state of a package containing a medical device over the period of time, determining, with the at least one processor, based on the state of the package over the period of time, whether the medical device is removed from the package, and determining, with the at least one processor, based on a determination that the medical device is removed from the package, at least a portion of the VAM data associated with the vascular access treatment associated with the patient.

In accordance with an embodiment of the present invention, the method further includes measuring, with a force sensor connected to a needleless connector including a fluid flow path, a force signal, receiving, with at least one processor, from the force sensor, the force signal, and determining, with the at least one processor, based on the force signal, at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof, and determining, with the at least one processor, based on the at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof, at least a portion of the VAM data associated with the vascular access treatment associated with the patient.

In accordance with an embodiment of the present invention, the force sensor is positioned between an outer surface of an inner wall of the needleless connector defining the fluid flow path of the needleless connector and an inner surface of an outer wall of the needleless connector surrounding the inner wall of the needleless connector.

In accordance with an embodiment of the present invention, wherein a first end of the needleless connector includes a septum including a surface facing in a first direction, wherein at least one of the force sensors is configured to detect a force in a second direction perpendicular to the surface of the septum facing in the first direction, and wherein the method further includes determining, with the at least one processor, based on the force signal indicating periodic forces in the second direction perpendicular to the surface of the septum facing in the first direction, the flushing event, wherein the flushing event includes a pulsatile flushing event.

In accordance with an embodiment of the present invention, the method further includes measuring, with a force sensor of a needleless connector including a fluid flow path, the force sensor, and a visual indicator, a force signal, receiving, with the at least one processor, from the force sensor, a force signal, determining, with the at least one processor, based on the force signal, at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof, and controlling, with the at least one processor, the visual indicator to provide a visual indication associated with the at least one of: the scrubbing event in which the needleless connector is scrubbed with the disinfectant, the flushing event in which the needleless connector is flushed with the solution, the connection event in which the needleless connector is connected to the medical device, the disconnection event in which the needleless connector is disconnected from the medical device, or any combination thereof.

In accordance with an embodiment of the present invention, the method further includes measuring, with an acoustic sensor connected to a needleless connector including a fluid flow path, a signal including a sound signature, receiving, with the at least one processor, from the acoustic sensor, a signal including a sound signature, determining, with the at least one processor, based on the signal, an event associated with the needleless connector, and determining, with the at least one processor, based on the determined event associated with the needleless connector, at least a portion of the VAM data associated with the vascular access treatment associated with the patient.

In accordance with an embodiment of the present invention, the method further includes measuring, with an optical sensor connected to a needleless connector including a fluid flow path and a septum, a movement of the septum, receiving, with the at least one processor, from the optical sensor, a signal associated with the movement of the septum, determining, with the at least one processor, based on the signal, an event associated with the needleless connector, and determining, with the at least one processor, based on the determined event associated with the needleless connector, at least a portion of the VAM data associated with the vascular access treatment associated with the patient.

### BRIEF DESCRIPTION OF THE DRAWINGS

FIG. 1A is a diagram of non-limiting embodiments or aspects of an environment in which systems, devices, products, apparatus, and/or methods, described herein, can be implemented;

FIG. 1B is diagram of non-limiting embodiments or aspects of components of a local system of FIG. 1A;

FIG. 2 is a diagram of non-limiting embodiments or aspects of components of one or more devices and/or one or more systems of FIGS. 1A and 1B;

FIGS. 3A and 3B are a diagram of non-limiting embodiments or aspects of an implementation of a management system and a local system;

FIG. 4 is a flowchart of non-limiting embodiments or aspects of a process for vascular access management;

FIG. 5 is a flowchart of non-limiting embodiments or aspects of a process for vascular access management;

FIGS. 6A-6C are diagrams of an overview of non-limiting embodiments or aspects of an implementation 600 relating to a process for vascular access management;

FIG. 7 is a diagram of non-limiting embodiments or aspects of an implementation of an environment of a local system in which systems, devices, products, apparatus, and/or methods, described herein, can be implemented;

FIG. 8 is a perspective view of non-limiting embodiments or aspects of example implementations of a medical device;

FIG. 9 is a perspective view of non-limiting embodiments or aspects of implementations of identifier elements;

FIG. 10 is a perspective view of non-limiting embodiments or aspects of implementations of identifier elements;

FIG. 11 illustrates an example visual representation of the implementation of the environment of the local system of FIG. 7;

FIG. 12 is a flowchart of non-limiting embodiments or aspects of a process for obtaining VAM data;

FIG. 13 is a flowchart of non-limiting embodiments or aspects of a process for obtaining VAM data;

FIG. 14 is a flowchart of non-limiting embodiments or aspects of a process for obtaining VAM data;

FIG. 15 is a flowchart of non-limiting embodiments or aspects of a process for obtaining VAM data;

FIGS. 16A and 16B are a flowchart of non-limiting embodiments or aspects of a process for obtaining VAM data;

FIG. 17 is a flowchart of non-limiting embodiments or aspects of a process for obtaining VAM data;

FIG. 18 is a flowchart of non-limiting embodiments or aspects of a process for obtaining VAM data;

FIG. 19 is a perspective view of non-limiting embodiments or aspects of a scrubbing event;

FIG. 20 is a perspective view of non-limiting embodiments or aspects of a syringe including a first identifier element associated with a plunger of the syringe and a second identifier element associated with a barrel of the syringe;

FIG. 21 is a diagram of non-limiting embodiments or aspects of an implementation of an environment in which systems, devices, products, apparatus, and/or methods, described herein, can be implemented;

FIGS. 22A-22C are diagrams of non-limiting embodiments or aspects of an implementation of one or more systems and/or one or more devices of FIG. 1;

FIG. 23 is a perspective view of non-limiting embodiments or aspects of an implementation of a smart device;

FIG. 24A is a side view of non-limiting embodiments or aspects of an implementation of a needleless connector;

FIG. 24B is a side view of non-limiting embodiments or aspects of an implementation of a smart device and a needleless connector;

FIG. 24C is a side view of non-limiting embodiments or aspects of an implementation of a smart device and a needleless connector;

FIG. 25A is a perspective view of non-limiting embodiments or aspects of an implementation of a smart device and a needleless connector;

FIG. 25B is a top view of non-limiting embodiments or aspects of an implementation of a smart device and a needleless connector;

FIG. 25C is a graph of non-limiting embodiments or aspects of a force signal over time;

FIGS. 26A and 26B show non-limiting embodiments or aspects of output of one or more systems and/or one or more devices of FIG. 1;

FIG. 27 is a diagram of non-limiting embodiments or aspects of an implementation of a smart device for detecting an extravasation and/or an infiltration of a medication in a catheter;

FIG. 28 is a flowchart of non-limiting embodiments or aspects of a process for identifying a lumen;

FIG. 29 is a flowchart of non-limiting embodiments or aspects of a process for identifying a lumen;

FIG. 30 is a flowchart of non-limiting embodiments or aspects of a process for locating a needle tip;

FIG. 31 is a flowchart of non-limiting embodiments or aspects of a process for event monitoring;

FIG. 32 is a side view of non-limiting embodiments or aspects of an implementation of a syringe; and

FIGS. 33A-33C are perspective and side views of non-limiting embodiments or aspects of an implementation of a disinfectant cap.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices that are configured to directly or indirectly communicate with or over one or more networks. A computing device may be a mobile or portable computing device, a desktop computer, a server, and/or the like. Furthermore, the term “computer” may refer to any computing device that includes the necessary components to receive, process, and output data, and normally includes a display, a processor, a memory, an input device, and a network interface. A “computing system” may include one or more computing devices or computers. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.). Further, multiple computers, e.g., servers, or other computerized devices directly or indirectly communicating in the network environment may constitute a “system” or a “computing system”.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc. In some non-limiting embodiments or aspects, satisfying a threshold may refer to recognition of a pattern in a signal as a result of a pattern recognition technique, a data mining technique, a slope of signal analysis, an Xbar R chart analysis, and/or the like being applied to the signal. For example, satisfying a threshold may be based on a dynamic time based analysis of a signal.

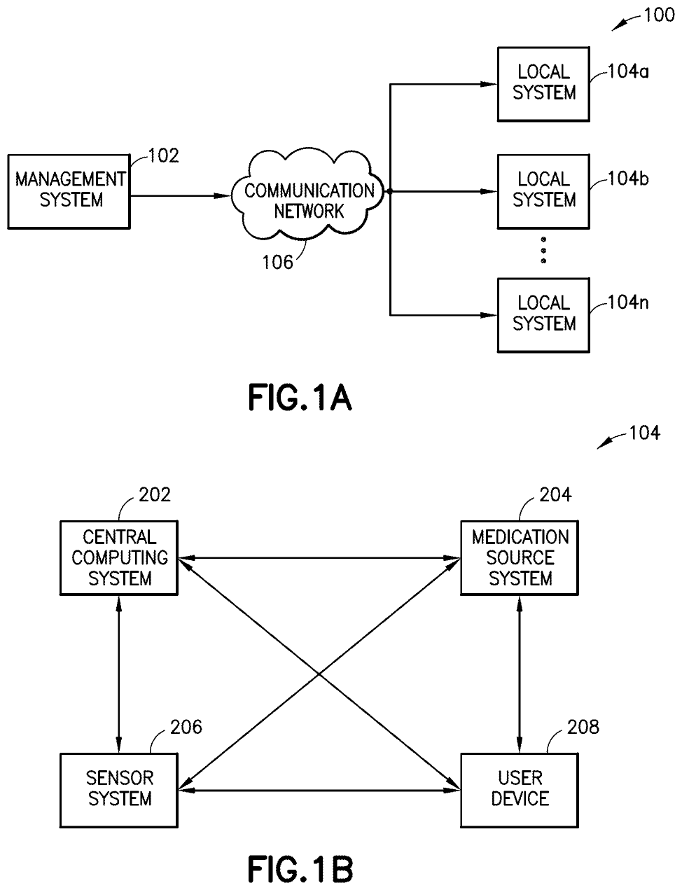

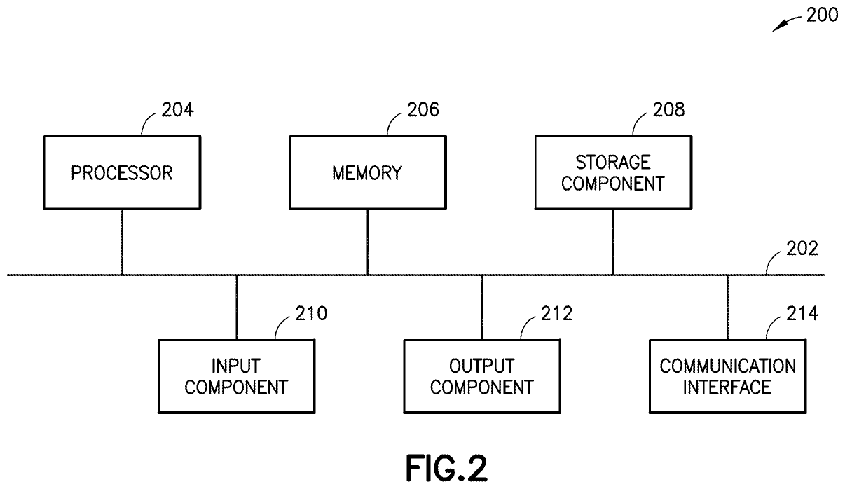

Referring now to FIG. 1A, FIG. 1A is a diagram of an example environment 100 in which systems, devices, products, apparatus, and/or methods described herein, may be implemented. As shown in FIG. 1A, environment 100 includes management system 102, a plurality of local systems 104a, 104b, . . . 104n, and/or communication network 106. Referring also to FIG. 1B, FIG. 1B is a diagram of non-limiting embodiments or aspects of a local system 104 of the plurality of local systems 104a, 104b, . . . 104n of FIG. 1A. As shown in FIG. 1B, local system 104 may include central computing system 202, medication source system 204, sensor system 206, and/or user device 208. Systems and/or devices of environment 100 and/or local system 104 may interconnect (e.g., communicate information and/or data, etc.) via wired connections, wireless connections, or a combination of wired and wireless connections (e.g., via communication network 106, etc.).

Management system 102 may include one or more devices capable of receiving information and/or data from the plurality of local systems 104a, 104b, . . . 104n (e.g., via communication network 106, etc.) and/or communicating information and/or data to the plurality of local systems 104a, 104b, . . . 104n (e.g., via communication network 106, etc.). For example, management system 102 may include one or more computing systems including one or more processors (e.g., one or more computing devices, one or more server computers, one or more mobile computing devices, etc.).

Management system 102 may be configured to access and/or update a standardized clinical protocol database located within management system 102 or external (e.g., remote from) management system 102. The standardized clinical protocol database may include clinical protocol data associated with standardized clinical protocols for vascular access management. In some non-limiting embodiments or aspects, a standardized clinical protocol may be customized according to a disease state of the patient, a type of local system (e.g., a care location, etc.) at which the patient is located, etc.

Local system 102 may include one or more devices capable of receiving information and/or data from management system 102 (e.g., via communication network 106, etc.) and/or communicating information and/or data to management system 102 (e.g., via communication network 106, etc.). For example, local system 102 may include one or more computing systems including one or more processors (e.g., one or more computing devices, one or more server computers, one or more mobile computing devices, etc.). In some non-limiting embodiments or aspects, local system 104 may include a home care system, an acute care system, a hospital care system, and/or the like. In such an example, local system 104 may include one or more signal extenders configured to extend wireless communication between components of local system 104, such as to extend wireless communication to cover an entire floor of a hospital enterprise, and/or the like.

Communication network 106 may include one or more wired and/or wireless networks. For example, communication network 106 may include a cellular network (e.g., a long-term evolution (LTE) network, a third generation (3G) network, a fourth generation (4G) network, a fifth generation (5G) network, a code division multiple access (CDMA) network, etc.), a short range wireless communication network (e.g., a Bluetooth network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

Central computing system 202 may include one or more devices capable of receiving information and/or data from management system 102, medication source system 204, sensor system 206, and/or user device 208 (e.g., via communication network 106, etc.) and/or communicating information and/or data to management system 102, medication source system 204, sensor system 206, and/or user device 208 (e.g., via communication network 106, etc.). For example, central computing system 202 may include one or more computing systems including one or more processors (e.g., one or more computing devices, one or more server computers, one or more mobile computing devices, etc.). In some non-limiting embodiments or aspects, central computing system 202 may be implemented within management system 102 medication source system 204, sensor system 206, and/or user device 208.

Medication source system 204 may include one or more devices capable of delivering one or more fluids to one or more lumens (e.g., fluid lines, IV lines, etc.). For example, medication source system 204 may include one or more manual fluid delivery systems (e.g., one or more IV bags, one or more syringes, etc.) and/or an infusion pump system including one or more infusion pumps.

Medication source system 204 may include one or more devices capable of receiving information and/or data from management system 102, central computing system 202, sensor system 206, and/or user device 208 (e.g., via communication network 106, etc.) and/or communicating information and/or data to management system 102, central computing system 202, sensor system 206, and/or user device 208 (e.g., via communication network 106, etc.). For example, medication source system 204 may include one or more computing systems including one or more processors (e.g., one or more computing devices, one or more server computers, one or more mobile computing devices, etc.).

Sensor system 106 may include one or more sensors configured to determine (e.g., determine, collect, acquire, capture, measure, sense, etc.) sensor data associated with a patient and/or a medical device. For example, sensor system 106 may include image capture system 702, one or more smart devices 804, and/or user device(s) 208.

Sensor system 206 may include one or more devices capable of receiving information and/or data from management system 102, central computing system 202, medication source system 204, and/or user device 208 (e.g., via communication network 106, etc.) and/or communicating information and/or data to management system 102, central computing system 202, medication source system 204, and/or user device 208 (e.g., via communication network 106, etc.). For example, sensor system 206 may include one or more computing systems including one or more processors (e.g., one or more computing devices, one or more server computers, one or more mobile computing devices, etc.).

User device 208 may include one or more devices capable of receiving information and/or data from management system 102, central computing system 202, medication source system 204, and/or sensor system 206 (e.g., via communication network 106, etc.) and/or communicating information and/or data to management system 102, central computing system 202, medication source system 204, and/or sensor system 206 (e.g., via communication network 106, etc.). For example, user device 208 may include one or more computing systems including one or more processors (e.g., one or more computing devices, one or more server computers, one or more mobile computing devices, etc.).

In some non-limiting embodiments or aspects, user device 208 includes a nurse station or terminal in a hospital. For example, user device 208 may provide bedside nurse support (e.g., recordation of events in real-time by a nurse and feedback to a nurse if events, such as scrubbing or flushing, are determined to be due or needed, etc.), nursing station manager support (e.g., optimization of flushing procedures to reduce workflow and improve timed targets for flushing, etc.), retrospective reporting for nursing administration (e.g., a scrub duration, a flushing technique, a time between flushes, improper medical device reuse, proper medical device replacement, etc.), and/or the like.

The number and arrangement of systems and devices shown in FIGS. 1A and 1B are provided as an example. There can be additional systems and/or devices, fewer systems and/or devices, different systems and/or devices, or differently arranged systems and/or devices than those shown in FIGS. 1A and 1B. Furthermore, two or more systems or devices shown in FIGS. 1A and 1B can be implemented within a single system or a single device, or a single system or a single device shown in FIGS. 1A and 1B can be implemented as multiple, distributed systems or devices. Additionally, or alternatively, a set of systems or a set of devices (e.g., one or more systems, one or more devices, etc.) of environment 100 and/or local system 104 can perform one or more functions described as being performed by another set of systems or another set of devices of environment 100 and/or local system 104.

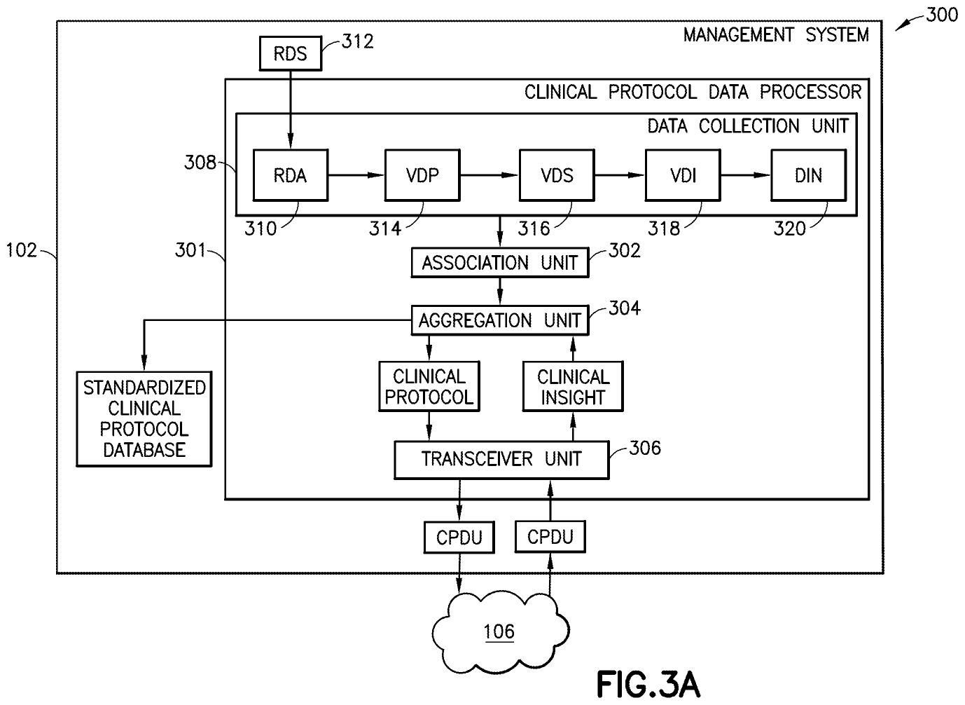

Referring now to FIG. 2, FIG. 2 is a diagram of example components of a device 200. Device 200 may correspond to one or more devices of management system 102, one or more devices local system 104, one or more devices of central computing system 202, one or more devices of medication source system 204, one or more devices of sensor system 206, and/or user device 208 (e.g., one or more devices of a system of user device 208, etc. In some non-limiting embodiments or aspects, one or more devices of management system 102, one or more devices local system 104, one or more devices of central computing system 202, one or more devices of medication source system 204, one or more devices of sensor system 206, and/or user device 208 (e.g., one or more devices of a system of user device 208, etc.) may include at least one device 200 and/or at least one component of device 200.

As shown in FIG. 2, device 200 may include bus 202, processor 204, memory 206, storage component 208, input component 210, output component 212, and/or communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally or alternatively, input component 210 may include a sensor for sensing information (e.g., a global positioning system (GPS) component, an accelerometer, a gyroscope, an actuator, a force sensor a camera, and/or any of the sensors described herein, etc.). Output component 212 may include a component that provides output information from device 200 (e.g., a display, a speaker, a tactile or haptic output, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardware circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, etc.). Device 200 may be capable of receiving information from, storing information in, communicating information to, or searching information stored in the data storage or one or more data structures in memory 206 and/or storage component 208.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

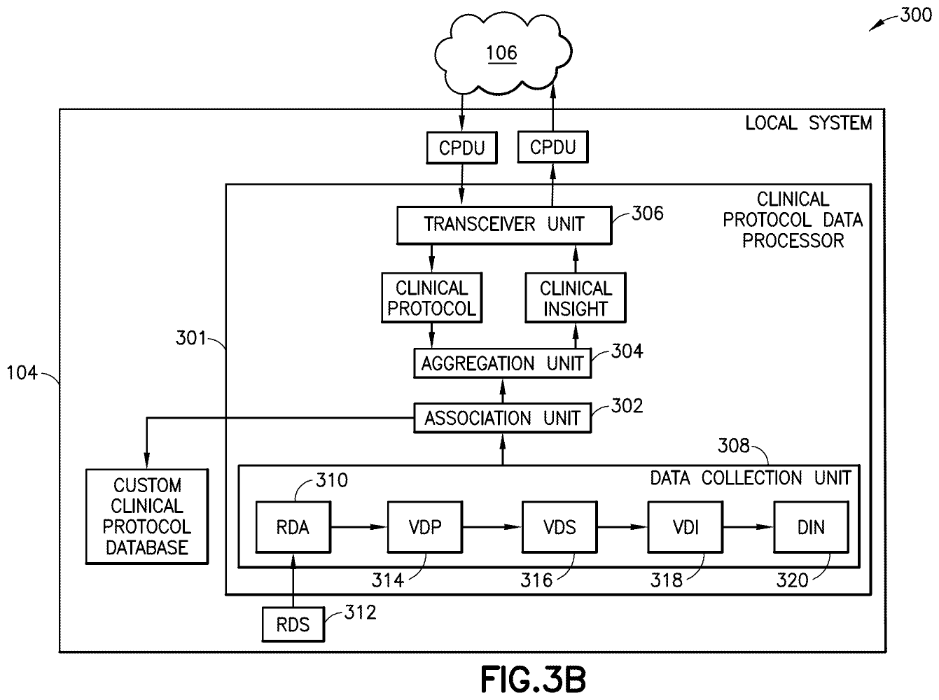

Referring now to FIGS. 3A and 3B, FIGS. 3A and 3B are a diagram of non-limiting embodiments or aspects of an implementation 300 of management system 102 and local system 104. As shown in FIGS. 3A and 3B, management system 102 may be configured as a central unit or command center for remotely monitoring line maintenance activities (e.g., flushing, scrubbing, medication delivery etc.) using VAM data received from the plurality of local systems 104a, 104b, . . . 104n, maintaining and enriching standardized clinical protocols, acquiring clinical insights according to the standardized clinical protocols, performing automatic registration of medical devices, and/or performing automatic registration of patients. In some non-limiting embodiments or aspects, management system 102 may store one or more interoperable license modules (e.g., protocols, clinical insights, etc.) that enable devices of different types (e.g., a device from a first manufacturer and a device from a second manufacturer different than the first manufacturer, etc.) to be connected and/or communicate with each other and/or management system 102 and/or local system 104.

Communication between management system 102 and local system 104 may be based on a clinical protocol data unit (CPDU). A CPDU may include a block of VAM data and/or clinical information that can be transferred over communication network 106. For example, a CPDU may include clinical protocol-specific information and/or a payload of VAM data. As an example, management system 102 may be configured to associate, aggregate, and/or transmit VAM data (e.g. meaningful and clinically relevant insights with a time stamp, etc.) as CPDUs over communication network 106 to local system 104 (e.g., to central computing system 202, etc.) and receive VAM data (e.g., sensor data or signals, patient data, user input data, etc.) as CPDUs over communication network 106 from local system 104.

Still referring to FIGS. 3A and 3B, management system 102 and/or local system 104 (e.g., central computing system 202, etc.) may each respectively include a clinical protocol data processor 301 including association unit 302, aggregation unit 304, transceiver unit 306, and/or data collection unit 308.

Association unit 302 may be programmed and/or configured to use one or more algorithms to associate VAM data and/or patient data with clinical standards data to determine one or more clinical insights and/or to associate the one or more clinical insights with VAM data to determine one or more clinical protocols. In some non-limiting embodiments or aspects, association unit 302 may generate hospital and/or patient specific custom clinical protocols from standard clinical protocols using VAM data and/or clinical insights. In some non-limiting embodiments or aspects, association unit 302 may use standard clinical protocols as is (e.g., without modifying the standard clinical protocols, etc.) depending upon a clinical condition.

Aggregation unit 304 may be programmed and/or configured to aggregate VAM data from a plurality of different data sources (e.g., from various smart devices, from a nursing table, from an EMR, etc.). Aggregation unit 304 may be programmed and/or configured to aggregate data from different association units 302. For example, aggregation unit 304 may aggregate VAM data from the plurality of different sources after the data is collected by data collection unit 308 and associated with clinical standards data by association unit 302.

Transceiver unit 306 may be programmed and/or configured to transmit and/or receive CPDUs over communication network 106, packetize VAM data, clinical protocols, and/or insights into CPDUs, and/or de-packetize CPDUs into VAM data, clinical protocols and/or insights. For example, transceiver unit 306 may transmit the data after the data has been aggregate by aggregation unit 304.

Data collection unit 308 may include raw data aggregator 310, raw data source(s) 312, VAM data pre-processor 314, VAM data source(s) 316, VAM data integrator 318, and/or VAM data input 320. Data collection unit 308 may be programmed and/or configured to collect source data (e.g., VAM data, patient data, etc.) from a plurality of different data sources (e.g., from various smart devices, from a nursing table, from an EMR, etc.). For example, a sequence of operation or data processing in data collection unit 308 may be from raw data source(s) 312 to raw data aggregator 310 to VAM data pre-processor 310 to VAM data source(s) 316 to VAM data integrator 318 to VAM data input 320. For example, a sequence of operation or data processing in data collection unit 308 may be from data collection unit 308 to association unit 302, to aggregation unit 304, to transceiver unit 306.

Raw data aggregator (RDA) 310 may be programmed and/or configured to interface with all available data sources, such as an EHR, a smart device system, treatment checklists, physician and nursing notes, assessment charts, product information, the clinical protocol database, and/or the like.

Raw data source(s) (RDS) 312 may include at least one of the following data sources: an EHR, a smart device system, treatment checklists, physician and nursing notes, assessment charts, product information, the clinical protocol database, or any combination thereof.

VAM data pre-processor (VDP) 314 may be programmed and/or configured to transform raw data and filter the data for vascular access treatment relevant information. For example, VAM data pre-processor 314 may be programmed and/or configured to filter vascular access treatment relevant data from each data source, normalize attributes in the filtered data, provide treatment of missing values, and/or perform feature engineering for better understanding by signal models.

VAM data source(s) (VDS) 316 may include data sources that include only vascular access treatment specific information. For example VAM data source(s) 316 may include vascular Access data derived from an EHR, vascular access product/practice data from a smart device system, physician and nursing notes converted to structured format using NLP, charts from VAM assessments, vascular signal data, vascular relevant data from treatment checklists, or any combination thereof.

VAM data integrator (VDI) 318 may be programmed and/or configured to combine preprocessed data from various sources into a single data source.

VAM data input (DIN) 320 may include a consolidated input or data structure with VAM data in which each row represent a single historical instance of a patient.

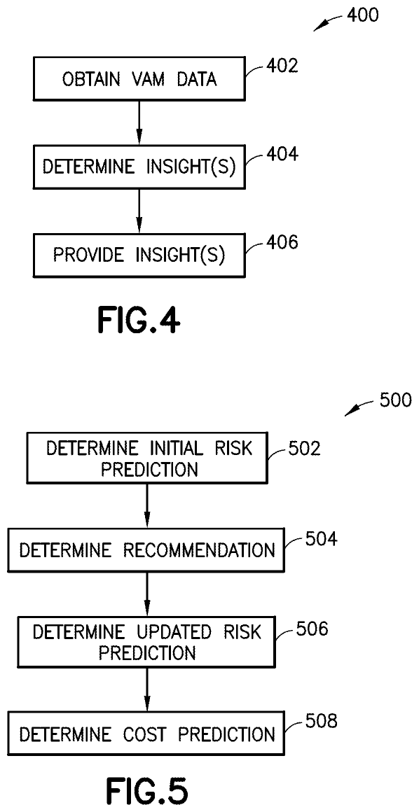

Referring now to FIG. 4, FIG. 4 is a flowchart of non-limiting embodiments or aspects of a process 400 for vascular access management. In some non-limiting embodiments or aspects, one or more of the steps of process 400 may be performed (e.g., completely, partially, etc.) by management system 102 (e.g., one or more devices of management system 102, etc.). In some non-limiting embodiments or aspects, one or more of the steps of process 400 may be performed (e.g., completely, partially, etc.) by another device or a group of devices separate from or including management system 102, such as local system 104 (e.g., one or more devices of local system 104, etc.), central computing system 202 (e.g., one or more devices of central computing system 202, etc.), medication source system 204 (e.g., one or more devices of medication source system 204, etc.), sensor system 206 (e.g., one or more devices of sensor system 206, etc.), and/or user device 208 (e.g., one or more devices of a system of user device 208, etc.).

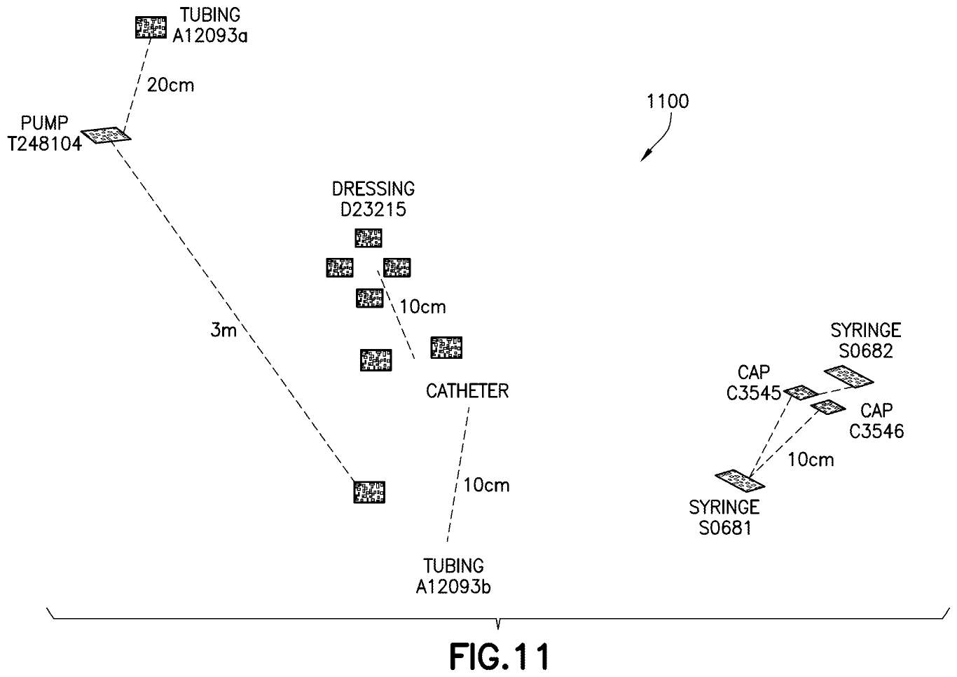

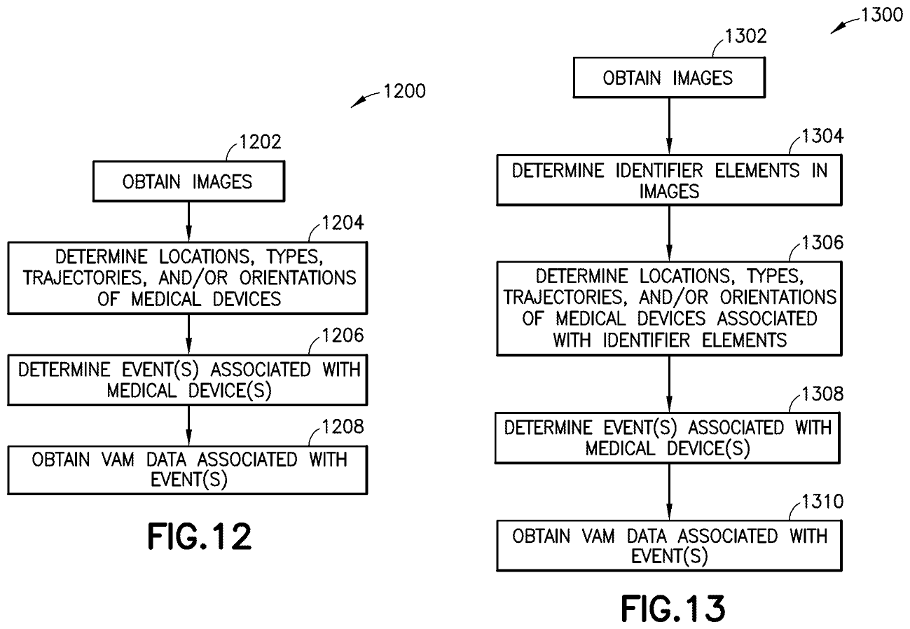

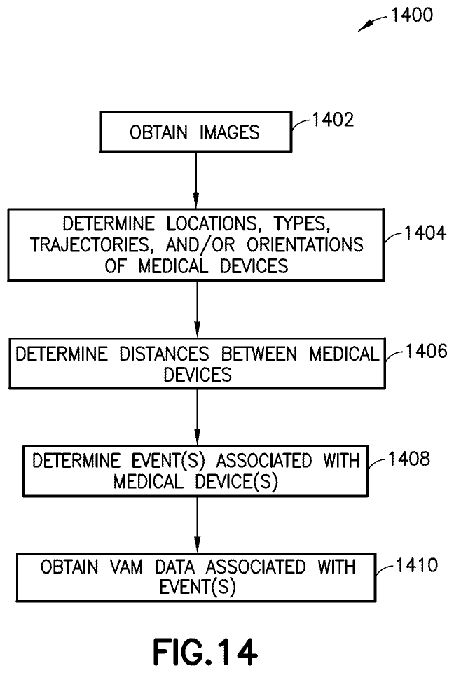

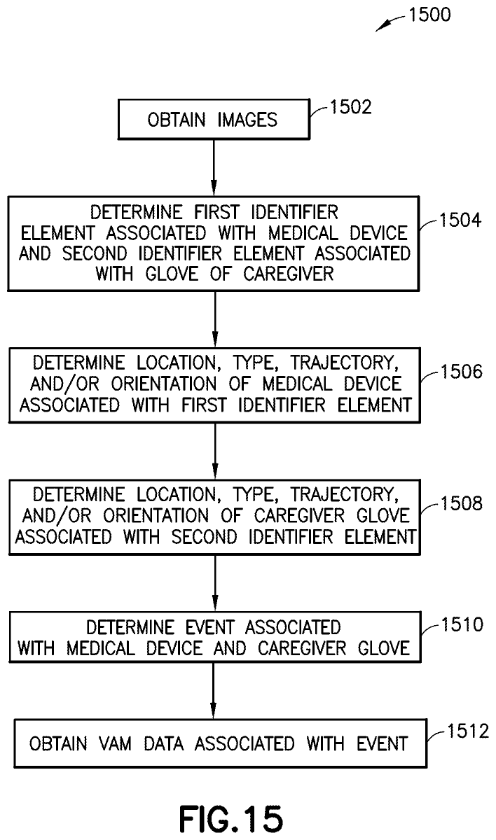

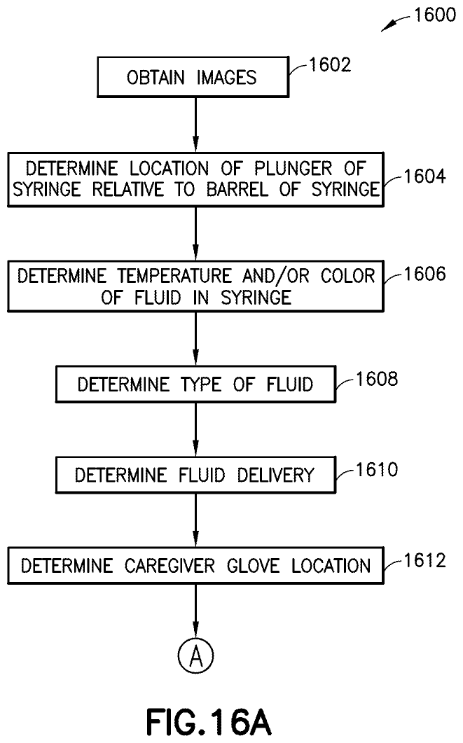

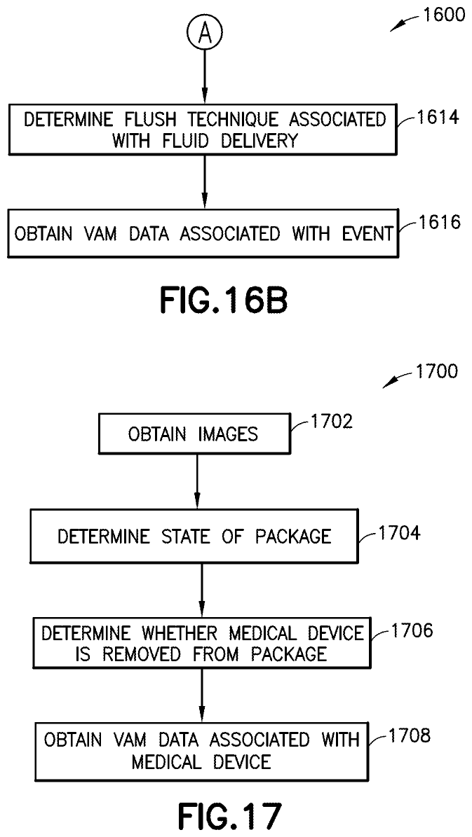

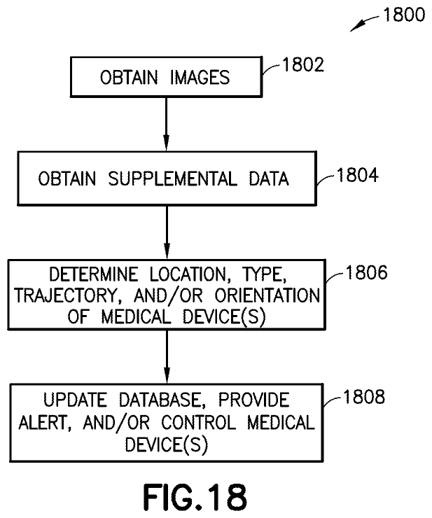

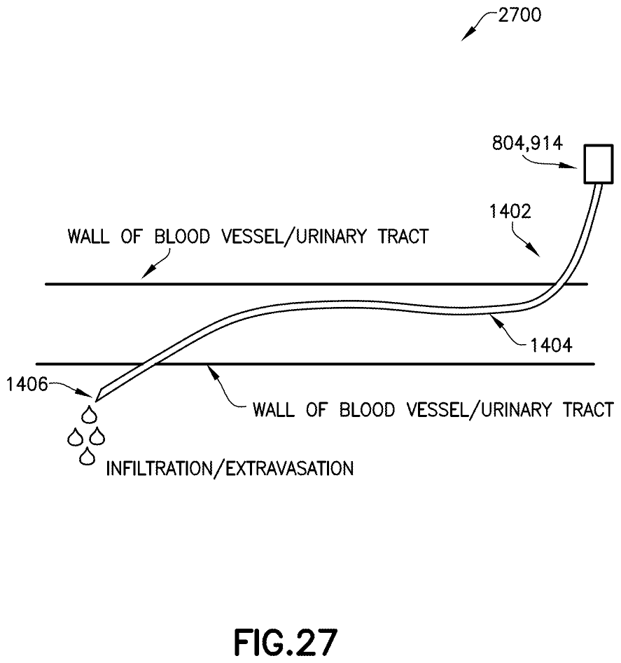

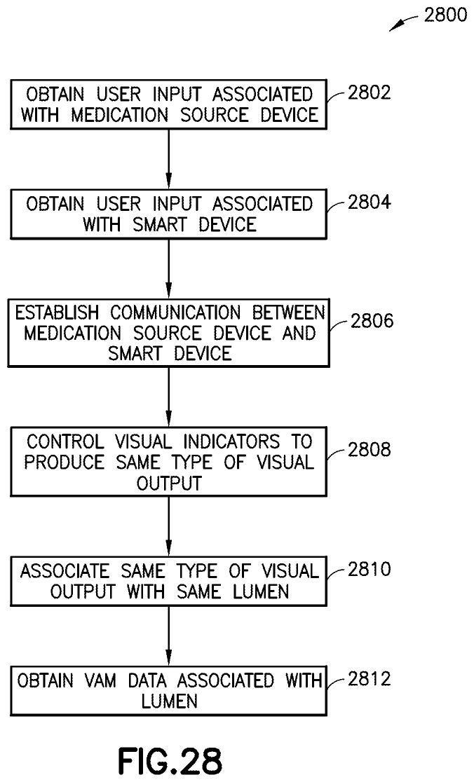

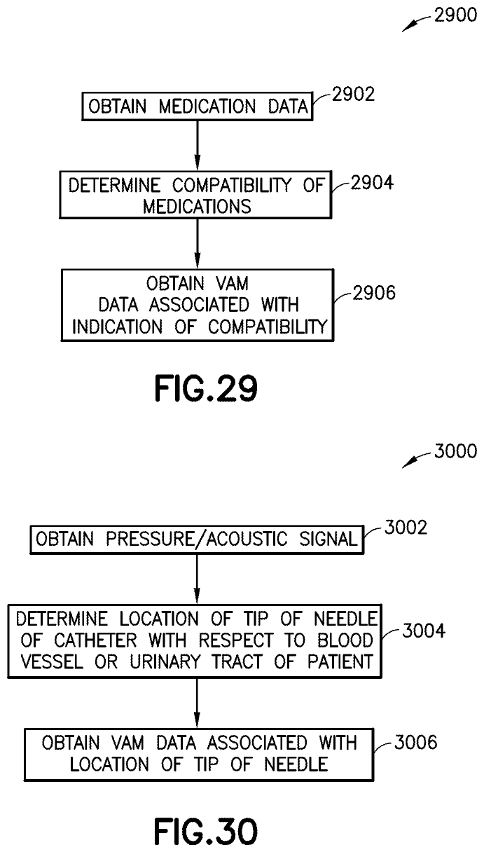

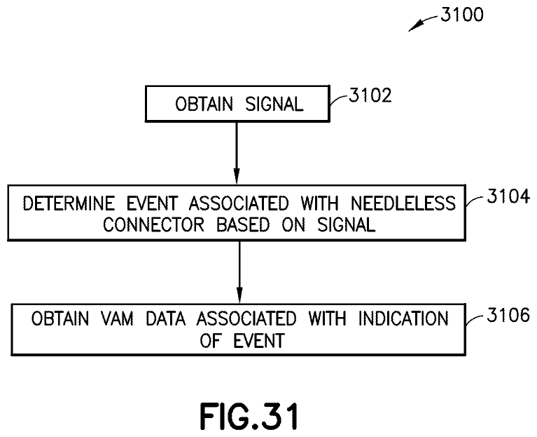

As shown in FIG. 4, at step 402, process 400 includes obtaining VAM data. For example, management system 102 may obtain VAM data associated with a vascular access treatment associated with a patient. As an example, management system 102 may obtain VAM data before, during, and/or after the vascular access treatment is provided to the patient. In such an example, management system 102 may receive and/or retrieve VAM data from at least one of the following: local system 104, central computing system 202, medication source system 204, sensor system 206, and/or user device 208, a hospital information system (HIS), an electronic medical record (EMR), an electronic health record (EHR), or any combination thereof. VAM data may also be obtained via interaction with a user interface of local system 104, central computing system 202, medication source system 204, sensor system 206, and/or user device 208 that may prompt a user and/or the patient for and/or record such data. For example, the VAM data obtained by management system 102 (and/or central computing system 202, etc.) may include any information, data, and/or signals obtained, received, retrieved, collected, measured, sensed, determined, provided, and/or transmitted as a part of one or more of the following processes described in more detail herein below: process 1200 in FIG. 12, process 1300 in FIG. 13, process 1400 in FIG. 14, process 1500 in FIG. 15, process 1600 in FIGS. 16A and 16B, process 1700 in FIG. 17, process 1800 in FIG. 18, process 2800 in FIG. 28, process 2900 in FIG. 29, process 3000 in FIG. 30, process 3100 in FIG. 31, or any combination thereof.

VAM data may include sensor data, user input data, patient data, medical device data, medication data, event data, compatibility data, location data, insight data, and/or clinical protocol data. For example, VAM data may include data associated with one or more vascular access treatments, such as EMR data, product data, caretaker notes, treatment checklist, sensor data, event data, VAM assessment, clinical protocols, and/or the like, associated with one or more patients. As an example, VAM data may include one or more of the following parameters: a patient identifier; a hospital identifier; a patient name; a patient gender; a patient age, a co-morbidity/problem associated with a patient (e.g., poor venous condition, etc.); a medication associated with a patient, a symptom associated with a patient; a reason for admission associated with a patient (e.g., surgery, etc.); an infusion type associated with a patient (e.g., non-vesicant, vesicant, etc.); an admission date associated with a patient; a readmission indicator (e.g., yes, no, etc.); a discharge date associated with a patient; a length of stay associated with a patient; a number of lines used associated with a patient; a type of accessories used associated with a patient (e.g., extension set with connector, etc.); a date of use associated with a medical device; an average dwell time, an average number of stick attempts, a complication (e.g., occlusion, no complication, etc.); a department; a nurse identifier; a nurse experience indicator (e.g., competent, expert, etc.); a question associated with a vascular access treatment (e.g., was the skin dried as per IFU, was the flush clamped before disconnecting, etc.); a question identifier associated with a question; an answer associated with a question (e.g., yes, no, etc.); a time stamp associated with a usage of a medical device; a device identifier associated with a medical device, a type of a medical device, a device signal associated with a medical device (e.g., scrubbed, vesicant infused, etc.); a section (e.g., a status, an observation, etc.); metadata and/or other keywords associated with a vascular access treatment entered by a user; a department (e.g., cardiology, radiology, etc.); a number of occlusion cases in a period of time, a number of CRBSI and/or CLABSI cases in a time period; a predicted vascular signal (e.g., CRBSI, phlebitis, etc.); an insight; an initial risk prediction; a recommendation; an updated risk prediction; a cost prediction; or any combination thereof.

Sensor data may include one or more parameters determined (e.g., determined, collected, acquired, captured, measured, sensed, etc.) by one or more sensors of sensor system 106 (e.g., an image capture system 702, smart device 804, user device 208, etc.). For example sensor data may include at least one of the following parameters: images and/or image data, determined events and/or event data, an identifier of a particular sensor, information, data, and/or a signal sensed, measured, and/or detected by one or more sensor in one or more smart devices and/or peripheral devices (e.g., a piezoelectric signal or data, a force signal or data, a flow, or any combination thereof. Sensor data may include patient data, medical device data, medication data, image data, and/or clinical protocol data.

User input data may include one or more parameters input via user interaction with a user interface of local system 104, central computing system 202, medication source system 204, sensor system 206, and/or user device 208. For example, user input data may include at least one of the following parameters: a number of stick attempts, a location of a stick attempt on a patient (e.g., left arm, right, arm, left leg, right leg, etc.), a location of an insertion site on a patient (e.g., left arm, right arm, left leg, right leg, etc.), a developmental venous anomaly (DVA), or any combination thereof. User input data may include patient data, medical device data, medication data, and/or clinical protocol data.

Patient data may include at least one of the following parameters associated with a patient: one or more patient demographics (e.g., age, weight, etc.), a treatment history, a patient identifier, or any combination thereof.

Medical device data may include at least one of the following parameters associated with a medical device: a device identifier, a type of a device, or any combination thereof.

A medical device may include a disposable medical device or a reusable medical device. For example, a medical device may include at least one of the following types of medical devices: a peripheral IV catheter (PIVC), a peripherally inserted central catheter (PICC), a midline catheter, a needleless connector, a catheter dressing, a catheter stabilization device, a disinfectant cap, a disinfectant swab or wipe, an IV tubing set, an infusion pump, a flush syringe, a medication delivery syringe, a caregiver glove, an IV fluid bag, a medication dispensing cabinet, an ultrasound device, a sharps collector, a smart device, or any combination thereof.

Medication data may include at least one of the following parameters associated with a medication: a type of the medication, a scheduled delivery of the medication via a particular medication source device and/or lumen, a previous delivery of the medication via a particular medication source device and/or lumen, an amount of the medication, a patient to which the medication is scheduled to be delivered (or delivered), one or more different types of medication that are incompatible for delivery via a same lumen with the medication, or any combination thereof.

Clinical protocol data may include at least one of the following parameters associated with a clinical protocol (e.g., standardized nursing care, practices, processes, and/or the like associated with a vascular access treatment, etc.): a catheter dwell time, a scrub time associated with a medical device, a scrub time, a flush time, a flush duration, a lock time, a lock duration, a disinfecting time, a disinfecting duration, an order of a plurality of clinical actions, or any combination thereof. For example, an example clinical protocol may include the following ordered instructions: 1. Scrub a hub to provide disinfection, 2. Flush to assess catheter functionality, 3. Scrub the hub to provide disinfection before delivering IV medication, 4. After delivering IV medication, scrub the hub to provide disinfection, 5. Flush to clear the medication, 6. Scrub the hub to provide disinfection, 7. Lock the catheter to maintain catheter patency, 8. Attach a disinfecting cap to disinfect between line accesses.

As shown in FIG. 4, at step 404, process 400 includes determining an insight associated with a vascular access treatment associated with a patient. For example, management system 102 may determine, based on VAM data, an insight associated with a vascular access treatment associated with a patient (e.g., insight data associated with an insight associated with a vascular access treatment associated with a patient, etc.). As an example, management system 102 may determine, based on the VAM data, before, during, and/or after the vascular access treatment is provided to the patient, an insight associated with the vascular access treatment.

An insight may include a data dashboard (e.g., graphs, trends, comparisons, etc.), a digitized audit, support and training information, a recommendation (including reasoning therefor), predictive analytics, and/or the like. For example, an insight may include patient's underlying condition and complication history at an early stage of treatment, associated risk of complications continuously updating during the entire course of patient treatment (e.g. an initial risk prediction, a reduced risk prediction, etc.), best practices and product recommendations (e.g., a recommendation, etc.), cost analysis based on adopted practices and products (e.g. a cost prediction, etc.), or any combination thereof. An insight may include metrics associated with a hospital or care location, such as a CRBSI and/or CLABSI Rate (outcome), an average dwell time length (outcome), a pull-through revenue, a number of recommendations adopted (adoption), a number and/or a type of products used for prepping an insertion site, a location of an insertion site on a patient, a number and/or a type of risk associated with an insertion site, and/or the like. Risk

Management system 102 may apply an algorithm or aspects of one or more algorithms, which may be an adaptation or implementation of a standardized clinical protocol, a professional society guideline, and/or a hospital procedure into computer code, to VAM data associated with one or more vascular access treatments associated with one or more patients to determine one or more insights associated with the one or more vascular access treatments. In such an example, different hospitals, locations, and/or providers may have different algorithms or aspects of one or more algorithms based on a local preference, a practice, a country, and/or other factors associated with the different hospitals.

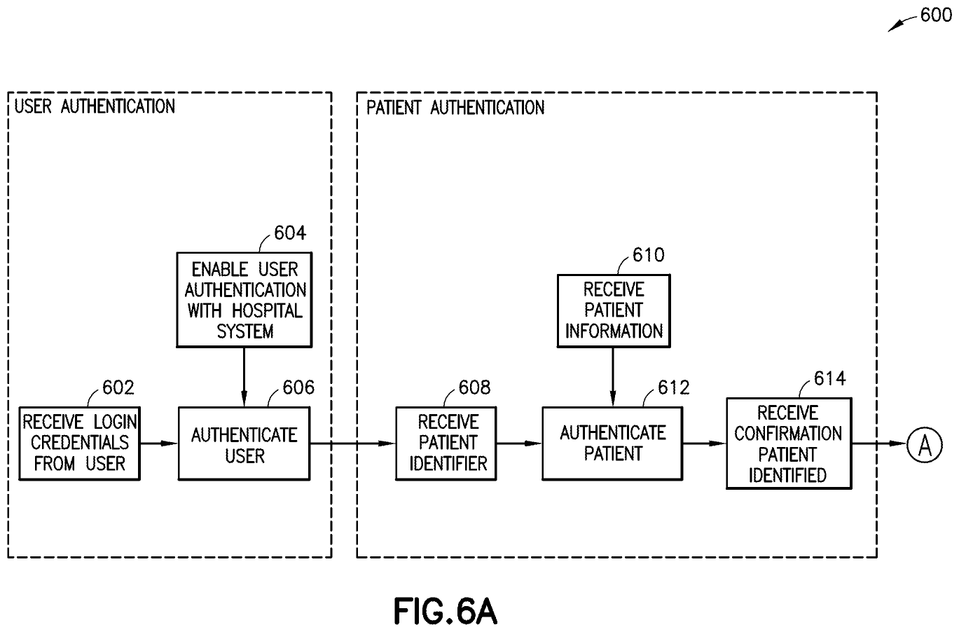

Further details regarding non-limiting embodiments or aspects of step 404 of process 400 are now provided with regard to FIG. 5. FIG. 5 is a flowchart of non-limiting embodiments or aspects of a process 500 for vascular access management. In some non-limiting embodiments or aspects, one or more of the steps of process 500 may be performed (e.g., completely, partially, etc.) by management system 102 (e.g., one or more devices of management system 102, etc.). In some non-limiting embodiments or aspects, one or more of the steps of process 500 may be performed (e.g., completely, partially, etc.) by another device or a group of devices separate from or including management system 102, such as local system 104 (e.g., one or more devices of local system 104, etc.), central computing system 202 (e.g., one or more devices of central computing system 202, etc.), medication source system 204 (e.g., one or more devices of medication source system 204, etc.), sensor system 206 (e.g., one or more devices of sensor system 206, etc.), and/or user device 208 (e.g., one or more devices of a system of user device 208, etc.).

As shown in FIG. 5, at step 502, process 500 includes determining an initial risk prediction associated with a vascular access treatment associated with a patient. For example, management system 102 may determine, based on VAM data, an initial risk prediction for a vascular access treatment to be administered to a patient. As an example, an initial risk prediction may include a probability that a patient experiences at least one complication in response to a vascular access treatment. Such risk prediction may be numerical from 0 to 100% or bucketed, for example, into low, medium, and high buckets.

A complication may include at least one of the following complications: a phlebitis, an occlusion, an infiltration, a Catheter Related Blood Stream Infection (CRBSI), a Central Line-associated Bloodstream Infection (CLABSI), a first stick failure in a right arm of a patient, a first stick failure in a left arm of a patient, a dislodgement of a catheter, an extravasation, an infiltration, or any combination thereof.

In some non-limiting embodiments or aspects, management system 102 may process VAM data associated with a vascular access treatment associated with a patient with a machine learning model to determine an initial risk prediction for the vascular access treatment associated with the patient. For example, management system 102 may generate a risk prediction model (e.g., an estimator, a classifier, a prediction model, a detector model, etc.) using machine learning techniques including, for example, supervised and/or unsupervised techniques, such as decision trees (e.g., gradient boosted decision trees, random forests, etc.), logistic regressions, artificial neural networks (e.g., convolutional neural networks, etc.), Bayesian statistics, learning automata, Hidden Markov Modeling, linear classifiers, quadratic classifiers, association rule learning, and/or the like. The risk prediction machine learning model may be trained to provide an output including a probability that a patient, in response to the vascular access treatment, experiences at least one complication in response to an input including the VAM data. In such an example, the risk prediction may include a probability score associated with a prediction that the patient experiences the at least one complication in response to the vascular access treatment.

Management system 102 may generate and/or update the risk prediction model based on VAM data (e.g., training data, etc.). In some implementations, the risk prediction model is designed to receive, as an input, VAM data (e.g., one or more parameters of the VAM data, EHR data, diagnostics, sensor data, real-time treatment checklists, a complication history associated with a patient, etc.) and provide, as an output, a prediction (e.g., a probability, a binary output, a yes-no output, a score, a prediction score, a classification, etc.) as to whether a patient experiences at least one complication in response to a vascular access treatment. In some non-limiting embodiments or aspects, management system 102 stores the risk prediction model (e.g., stores the model for later use). In some non-limiting embodiments or aspects, management system 102 stores the risk prediction model in a data structure (e.g., a database, a linked list, a tree, etc.). In some non-limiting embodiments, the data structure is located within management system 102 or external (e.g., remote from) management system 102 (e.g., within auxiliary system 112, etc.).

As shown in FIG. 5, at step 504, process 500 includes determining a recommendation associated with a patient for a vascular access treatment. For example, management system 102 may determine, based on VAM data and the initial risk predication, a recommendation associated with a vascular access treatment to be administered to and/or currently being administered to a patient. As an example, a recommendation may include a recommended process and/or a recommended product (e.g., best practices, etc.) to use for the vascular access treatment.

A recommended process may include at least one of the following recommended processes: a recommendation to use a specific arm of a patient for a vascular access treatment (e.g., a specific arm for a first stick, etc.), a recommendation to use a specific type of medical device for a vascular access treatment (e.g., a specific type of catheter, such as an ultrasound guided catheter, a specific type of syringe, such as a pre-filled saline syringe, etc.), a recommendation to disinfect a medical device (e.g., a recommendation to scrub a catheter hub for a period of time, etc.), a recommendation to flush and/or a type of flush to perform (e.g., a recommendation to use a pulsatile flush, etc.), a recommendation to lock a catheter, a recommendation to attach a disinfection cap, or any combination thereof.

A recommended product may include a recommendation to use a specific type of a medical device for a vascular access treatment. For example, recommended product may include a specific type of catheter (e.g., an ultrasound guided catheter, et.), a specific type of syringe (e.g., a pre-filled saline syringe, etc.), or any combination thereof. As an example, a recommended product may include one or more of the following: Peripheral IV Catheters: 1. Conventional straight and ported catheters, 2. Safety straight and ported catheters, 3. Conventional closed catheter systems, 4. Safety closed catheter systems, 5. Guidewire-assisted intravascular catheters; Catheter Care Syringes: 1. Saline sterile fluid path and externally sterile prefilled syringes, 2. Heparin prefilled syringes, 3. Citrate prefilled syringes, 4. Flush syringes with alcohol disinfectant pad; Medication Delivery Needles and Syringes: 1. Conventional hypodermic needles and syringes Blunt Fill/Filter Needles, Fluid Dispensing Syringes), 2. Safety hypodermic needles and syringes, 3. Reuse prevention syringes, 4. Enteral/Oral syringes, 5. Spinal and epidural needles; Advanced Access Devices: 1. Peripherally inserted central catheters, 2. Acute dialysis catheters, 3. Acute central venous catheters, 4. Midline catheters, 5. Port access devices, 6. Intraosseous devices; Other Catheter Care Devices: 1. Disinfecting caps, 2. Single-use skin preparation antiseptics, 3. Dressings and dressing change kits (Antimicrobial Hemostatic IV Dressing), 4. Stabilization devices; Other Medication Delivery Devices: 1. Regional anesthesia kits and trays, 2. Sharps collectors; or any combination thereof.

In some non-limiting embodiments or aspects, management system 102 may process VAM data and/or an initial risk prediction associated with a vascular access treatment associated with a patient with a k-nearest neighbors algorithm (k-NN) to determine a recommendation associated with the vascular access treatment associated with the patient. For example, management system 102 may identify the most similar patient characteristics and practices and/or products associated with the patients based on a distance metric and recommend products and/or processes for new patients based thereon.

In some non-limiting embodiments or aspects, management system 102 may process VAM data and/or an initial risk prediction associated with a vascular access treatment associated with a patient with a machine learning model to determine a recommendation associated with the vascular access treatment associated with the patient. For example, management system 102 may generate a recommendation model (e.g., an estimator, a classifier, a prediction model, a detector model, etc.) using machine learning techniques including, for example, supervised and/or unsupervised techniques, such as decision trees (e.g., gradient boosted decision trees, random forests, etc.), logistic regressions, artificial neural networks (e.g., convolutional neural networks, etc.), Bayesian statistics, learning automata, Hidden Markov Modeling, linear classifiers, quadratic classifiers, association rule learning, and/or the like. The recommendation machine learning model may be trained to provide an output including a recommendation that a recommended best practice and/or a recommended product be used for the vascular access treatment.

Management system 102 may generate and/or update the recommendation model based on VAM data and/or one or more previous initial risk predications (e.g., training data, etc.). In some implementations, the recommendation model is designed to receive, as an input, VAM data (e.g., one or more parameters of the VAM data, EHR data, diagnostics, sensor data, real-time treatment checklist, etc.) and an initial risk predication (e.g., a complication predicted for the patient, etc.) associated with a vascular access treatment and provide, as an output, a recommendation that a recommended best practice and/or a recommended product be used for the vascular access treatment. In some non-limiting embodiments or aspects, management system 102 stores the recommendation model (e.g., stores the model for later use). In some non-limiting embodiments or aspects, management system 102 stores the recommendation model in a data structure (e.g., a database, a linked list, a tree, etc.). In some non-limiting embodiments, the data structure is located within management system 102 or external (e.g., remote from) management system 102 (e.g., within auxiliary system 112, etc.).

As shown in FIG. 5, at step 506, process 500 includes determining an updated risk prediction associated with a patient for a vascular access treatment. For example, management system 102 may determine, based on VAM data updated based on a recommendation, an updated prediction for a vascular access treatment to be administered to a patient. As an example, an updated risk prediction may include a probability that a patient experiences at least one complication in response to a vascular access treatment that uses and/or is modified to use a recommendation(s) determined as described herein above with respect to step 504. In such an example, management system 102 may process VAM data associated with a vascular access treatment associated with a patient that has been updated based on a recommendation that a recommended best practice and/or a recommended product be used for the vascular access treatment with the risk predication model described herein above with respect to step 502 to determine an updated risk predication associated with the vascular access treatment associated with the patient.

As shown in FIG. 5, at step 508, process 500 includes determining a cost prediction associated with a vascular access treatment. For example, management system 102 may determine, based on VAM data, an initial risk predication, a recommendation, and/or an updated risk prediction, a cost prediction associated with the vascular access treatment associated with the patient. As an example, a cost prediction may include an overhead cost associated with each process and/or product associated with the vascular access treatment and/or a predicted savings in terms of a reduced cost of complication from adoption of a recommended process and/or a recommend product.

In some non-limiting embodiments or aspects, management system 102 may process VAM data associated with a vascular access treatment associated with a patient, an initial risk prediction associated with the vascular access treatment, and/or a recommendation associated with the vascular access treatment (e.g., an adopted recommendation, etc.) with a machine learning model to determine a cost prediction for the vascular access treatment associated with the patient. For example, management system 102 may generate a cost prediction model (e.g., an estimator, a classifier, a prediction model, a detector model, etc.) using machine learning techniques including, for example, supervised and/or unsupervised techniques, such as decision trees (e.g., gradient boosted decision trees, random forests, etc.), logistic regressions, artificial neural networks (e.g., convolutional neural networks, etc.), Bayesian statistics, learning automata, Hidden Markov Modeling, linear classifiers, quadratic classifiers, association rule learning, and/or the like. The cost prediction machine learning model may be trained to provide an output including a cost prediction associated with the vascular access treatment. In such an example, the cost prediction may include a probability score associated with a prediction of a cost associated with the vascular access treatment.

Management system 102 may generate and/or update the cost prediction model based on VAM data associated with a vascular access treatment associated with a patient, an initial risk prediction associated with the vascular access treatment, and/or a recommendation associated with the vascular access treatment (e.g., training data, etc.). In some implementations, the cost prediction model is designed to receive, as an input, VAM data associated with a vascular access treatment associated with a patient (e.g., one or more parameters of the VAM data, EHR data, diagnostics, sensor data, real-time treatment checklists), an initial risk prediction associated with the vascular access treatment (e.g., a predicted complication associated with the patient, etc.), and/or a recommendation associated with the vascular access treatment (e.g., an adopted recommendation, a process used for a vascular access treatment, a product used for a vascular access treatment, etc.) and provide, as an output, a prediction (e.g., a probability, a binary output, a yes-no output, a score, a prediction score, a classification, etc.) of a cost associated with the vascular access treatment (e.g., an overhead cost associated with each process and/or product associated with the vascular access treatment and/or a predicted savings in terms of reduced cost of complication from adoption of a recommended process and/or a recommend product, etc.). In some non-limiting embodiments or aspects, management system 102 stores the cost prediction model (e.g., stores the model for later use). In some non-limiting embodiments or aspects, management system 102 stores the cost prediction model in a data structure (e.g., a database, a linked list, a tree, etc.). In some non-limiting embodiments, the data structure is located within management system 102 or external (e.g., remote from) management system 102 (e.g., within auxiliary system 112, etc.).

As shown in FIG. 4, at step 406, process 400 includes providing an insight associated with a vascular access treatment associated with a patient. For example, management system 102 may provide an insight associated with a vascular access treatment associated with a patient. As an example, management system 102 may provide (e.g., via user device 208, etc.), before, during, and/or after the vascular access treatment is provided to the patient, the insight associated with the vascular access treatment associated with the patient.

In some non-limiting embodiments or aspects, providing an insight associated with a vascular access treatment associated with a patient may include automatically controlling a medical device associated with the vascular access treatment. For example, management system 102 may automatically control, based on VAM data associated with a vascular access treatment associated with a patient, an initial risk prediction associated with the vascular access treatment, a recommendation associated with the vascular access treatment, an updated risk prediction associated with the vascular access treatment, and/or an insight associated with the vascular access treatment, a medical device during the vascular access treatment provided to the patient. As an example, management system 102 may automatically control, based on VAM data associated with a vascular access treatment associated with a patient, an initial risk prediction associated with the vascular access treatment, a recommendation associated with the vascular access treatment, an updated risk prediction associated with the vascular access treatment, and/or an insight associated with the vascular access treatment, a valve and/or an infusion pump associated with the vascular access treatment to stop a flow of fluid in a fluid flow path including the valve and/or the infusion pump.

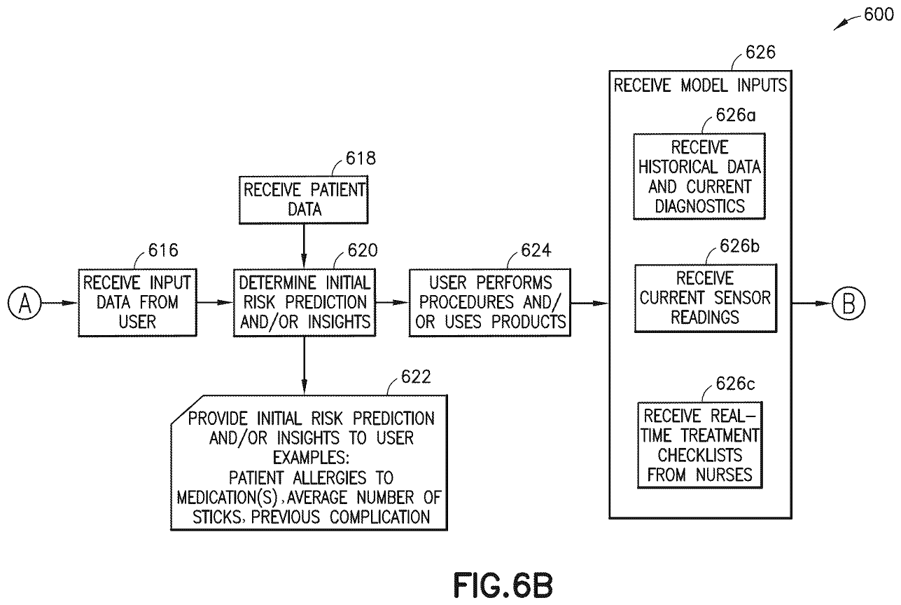

Referring now to FIGS. 6A-6C, FIGS. 6A-6C are diagrams of an overview of non-limiting embodiments or aspects of an implementation 600 relating to a process for vascular access management.

As shown by reference number 602 in FIG. 6A, central computing system 202 may receive, via user device 208, from a user (e.g., a nurse, etc.), login credentials (e.g., a username, a password, etc.) associated with the user. As shown by reference number 604 in FIG. 6A, management system 102 may enable user authentication with central computing system 202 (e.g., the hospital system, etc.). As shown by reference number 606 in FIG. 6A, central computing system 202 may authenticate the user.

As shown by reference number 608 in FIG. 6A, central computing system 202 may receive (e.g., via user device 208, etc.) a patient identifier associated with a patient. For example, a nurse may scan, with a scanner, a wristband of a patient to input the patient identifier to central computing system 202. As shown by reference number 610 in FIG. 6A, central computing system 202 may receive patient information and/or data associated with the patient. For example, central computing system 202 may receive an EMR associated with the patient. The EMR may contain historical patient data and/or VAM data associated with the patient. As shown by reference number 612 in FIG. 6A, central computing system 202 may authenticate the patient. As shown by reference number 614 in FIG. 6A, central computing system 202 may receive (e.g., via user device 208, etc.) a confirmation from the user that the patient is identified.

As shown by reference number 616 in FIG. 6B, central computing system 202 may receive (e.g., via user device 208, etc.) input data from the user. For example, central computing system 202 may receive patient data and/or VAM data associated with the patient (e.g., a patient temperature, a patient blood pressure, etc.) that is manually input by the user. As shown by reference number 618 in FIG. 6B, central computing system 202 may receive patient data associated with the patient from management system 102 and/or one or more databases associated with central computing system 202 and/or the hospital. As shown by reference number 620 in FIG. 6B, central computing system 202 (and/or management system 102) may determine an initial risk prediction and/or insights associated with a vascular access treatment to be performed on the patient. For example, central computing system 202 may determine a first set of insights and/or recommendations based on currently available VAM data associated with the patient. As shown by reference number 622 in FIG. 6B central computing system 202 may provide (e.g., via user device 208, etc.) the initial risk prediction and/or the insights to the user. As shown by reference number 624 in FIG. 6B, the user (e.g., the nurse, etc.) may perform a set of procedures and/or use products associated with the vascular access treatment.

As shown by reference number 626 in FIG. 6B, central computing system 202 (and/or management system 102) may receive model inputs (e.g., VAM data associated with the patient, VAM data associated with the vascular access treatment, etc.). For example, as shown by reference number 626a in FIG. 6B, central computing system 202 may receive historical data and current diagnostics associated with the patient and/or previous vascular access treatments for other patients. For example, as shown by reference number 626b in FIG. 6B, central computing system 202 may receive current sensor readings (e.g., from image capture system 702, from smart device(s) 804, from user device 208, etc.). For example, as shown by reference number 626c in FIG. 6B, central computing system 202 may receive one or more real-time treatment checklists (e.g., medical device data associated with products used, event data associated with events and/or procedures performed, etc.) from the user (e.g., via user device 208, etc.).

As shown by reference number 628 in FIG. 6C, central computing system 202 (and/or management system 102) may process the model inputs using models for risk prediction, recommendation, and cost analysis. As shown by reference number 630 in FIG. 6C, central computing system 202 (and/or management system 102) may provide model outputs as a result of processing the model inputs using models for risk prediction, recommendation, and cost analysis. For example, as shown by reference numbers 628a and 630a in FIG. 6C, central computing system 202 (and/or management system 102) may apply a risk prediction model to the model inputs to determine a probability of complications associated with the vascular access treatment for the patient. For example, as shown by reference numbers 628b and 630b in FIG. 6C, central computing system 202 (and/or management system 102) may apply a recommendation model to the model inputs to determine recommended products and practices for the vascular access treatment associated with the patient. For example, as shown by reference numbers 628c and 630c in FIG. 6C, central computing system 202 (and/or management system 102) may apply the risk prediction model to the model inputs and/or the recommended products and practices to determine an updated risk prediction (e.g., a probability of reduction of risks of complication for each recommended product and each recommended practice and/or combinations thereof, etc.). For example, as shown by reference numbers 628d and 630d in FIG. 6C, central computing system 202 (and/or management system 102) may apply a cost prediction engine to the model inputs, the initial risk prediction, and/or the recommended products and practices to determine an overhead cost associated with each product and process and a cost savings in terms of reduced risk of complication.

Still referring to FIGS. 6A-6C, an example of model inputs may include an indication that a patient has a difficult venous access (DVA), an average number of PIV stick attempts in a right arm of the patient of 2.5, a patient history of phlebitis, and a previous treatment cost $2000 plus $1000 due to the phlebitis. The risk prediction model may provide, based on these model inputs, an initial risk prediction including a probability of a first stick failure in the right arm of 70% and a probability of phlebitis of 20%. The recommendation model may provide, based on these model inputs and/or the initial risk prediction, a recommendation to use an ultrasound guided catheter and a recommendation to use a left arm of the patient. The risk prediction model may provide, based on these model inputs, the initial risk prediction, and/or the recommendations, an updated risk prediction including a probability of first stick failure in the left arm of 20% and a probability of phlebitis of 2%. The cost prediction engine may provide, based on these model inputs, the initial risk prediction, the recommendations, and/or the updated risk prediction, an overhead cost of the recommended processes and/or products of $5 and a cost savings in terms of reduced risk of $1000.

Another example of model inputs may include an indication that a patient has no vascular access history (e.g., a new patient, etc.), a scrubbing event for a catheter hub associated with an insufficient duration of scrubbing (e.g., 3 seconds, etc.), an indication that pulsatile flushing is not detected, and a cost of treatment for patients with a similar patient profile of $1500. The risk prediction model may provide, based on these model inputs, an initial risk prediction including a probability of CRBSI of 1% and a probability of occlusion of 15%. The recommendation model may provide, based on these model inputs and/or the initial risk prediction, a recommendation to scrub the catheter hub for at least 10 seconds, a recommendation to use a pulsatile flush, and a recommendation to use a pre-filled saline syringe. The risk prediction model may provide, based on these model inputs, the initial risk prediction, and/or the recommendations, an updated risk prediction including a probability of CRBSI of 0.001% and a probability of occlusion of 0.2%. The cost prediction engine may provide, based on these model inputs, the initial risk prediction, the recommendations, and/or the updated risk prediction, an overhead cost of the recommended processes and/or products of $1 and a cost savings in terms of reduced risk of $8000.

Accordingly, non-limiting embodiments or aspects of the present disclosure may help a medical practitioner to select a correct medical device (e.g., a correct catheter, etc.) for a vascular access treatment, properly prepare the skin of a patient for the vascular access treatment, properly place the medical device for the vascular access treatment, properly maintain the device for the vascular access treatment, properly use the device for the vascular access treatment, and/or properly secure the device for the vascular access treatment. In this way, non-limiting embodiments or aspects of the present disclosure may reduce vascular catheter colonization and catheter-related bloodstream infections (CRBSI) in patients with central venous or arterial catheters, assist hospitals in improving dwell times of peripheral IV catheters and in reducing vascular access complications in patients, reduce overall cost associated with vascular access complications, and/or the like.

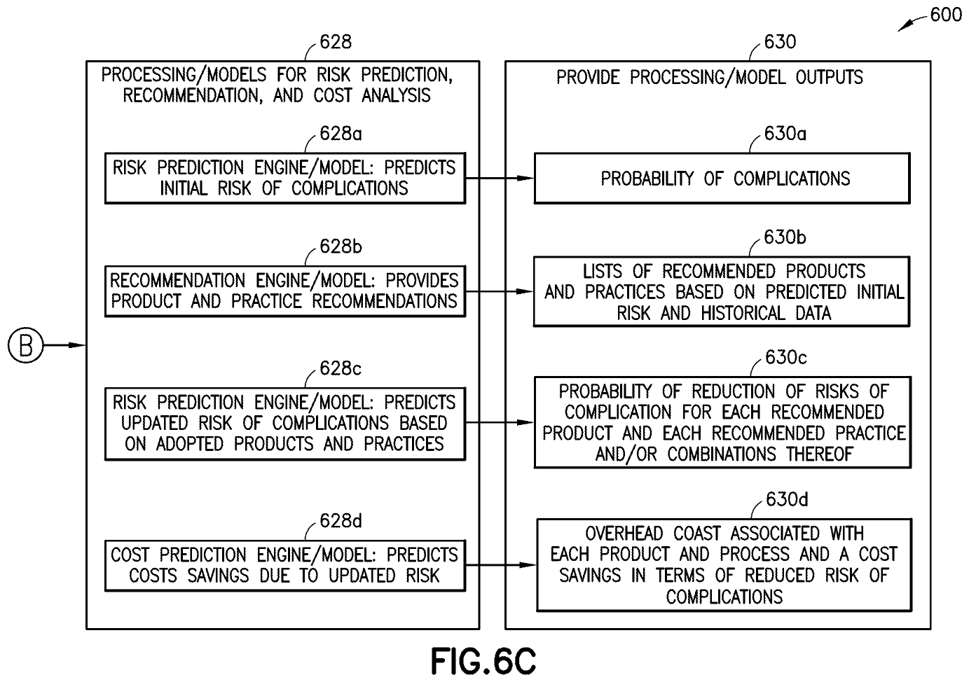

Referring now to FIG. 7, FIG. 7 is a diagram of non-limiting embodiments or aspects of an implementation of an environment 700 of a local system 104 in which systems, devices, products, apparatus, and/or methods, described herein, can be implemented. For example, as shown in FIG. 7, environment 700 may include a hospital room including a patient, image capture system 702, one or more medical devices 712, one or more identifier elements 714 associated with the one or more medical devices 712, and/or a caretaker (e.g., a nurse, etc.). As an example, and referring also to FIG. 2, sensor system 206 may include image capture system 702 and/or identifier elements 714.

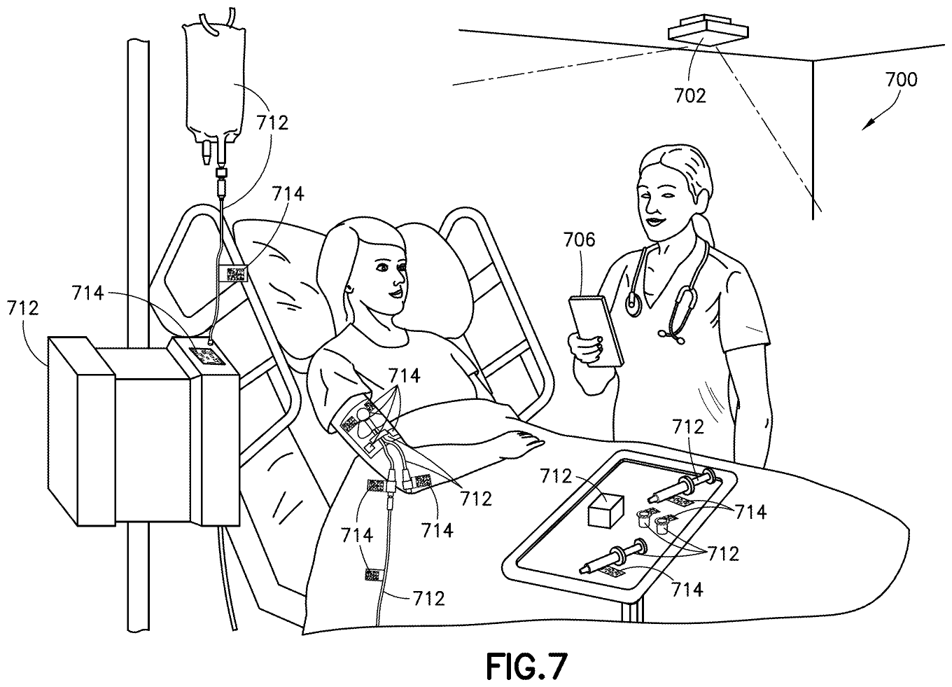

A medical device 712 may enter environment 700 (e.g., via the caretaker, etc.), remain in environment 700 for a period of time (or indefinitely) during which the medical device 712 may move within environment 700 and/or interact with (e.g., connect to, disconnect from, etc.) one or more other medical devices 712, the patient, and/or the caretaker, and/or exit environment 700 at a subsequent time after entering environment 700 (e.g., via the caretaker, etc.). A medical device 712 may include a disposable medical device and/or a reusable medical device. For example, a medical device 712 may include at least one of the following types of medical devices a peripheral IV catheter (PIVC), a peripherally inserted central catheter (PICC), a midline catheter, a needleless connector, a catheter dressing, a catheter stabilization device, a disinfectant cap, a disinfectant swab or wipe, an IV tubing set, an infusion pump, a flush syringe, a medication delivery syringe, a caregiver glove, an IV fluid bag, a medication dispensing cabinet, an ultrasound device, a sharps collector, or any combination thereof. FIG. 8 provides a perspective view of non-limiting embodiments or aspects of implementations of a medical device 712. As described in more detail herein below, in some non-limiting embodiments or aspects, image capture system 702 (e.g., sensor system 206, etc.), management system 102, and/or central computing system 202 may detect and use a shape, a size, a movement or trajectory, a location, and/or an orientation of a medical device 712 to identify a type of medical device 712 and/or to uniquely identify medical device 712 from other medical devices in environment 700, as well as to track locations of the medical device 712 in environment 700 and/or determine events associated with the medical device 712.

Detection of a shape, a size, a movement or trajectory, a location, an orientation, and/or the like of an object may be computationally expensive and/or error-prone. For example, a camera based object detection system may make a mistake in identifying similar objects and/or miss (e.g., fail to detect, etc.) an object in a noisy environment. Accordingly, in some non-limiting embodiments or aspects, an identifier element 714 (e.g., a tag, a label, a code, etc.) may be associated with (e.g., removably attached to, permanently attached to, integrated with, implemented on, etc.) a medical device 712. In some non-limiting embodiments or aspects, each medical device 712 in environment 700 may be associated with an identifier element 714. In some non-limiting embodiments or aspects, only a portion the medical devices 712 in environment 700 may be associated with identifier elements 714. In some non-limiting embodiments or aspects, none of the medical devices 712 in environment 700 may be associated with identifier elements 714. As described in more detail herein below, in some non-limiting embodiments or aspects, image capture system 702 (e.g., sensor system 206, etc.), management system 102, and/or central computing system 202 may detect and use a shape, a size, a movement or trajectory, a location, and/or an orientation of an identifier element to identify a type of medical device 712 associated with the identifier element 714 and/or to uniquely identify medical device 712 associated with the identifier element 714 from other medical devices in environment 700, as well as to track locations of the medical device 712 associated with the identifier element 714 in environment 700 and/or determine events associated with the medical device 712 associated with the identifier element 714.

An identifier element 714 may encapsulate an identifier associated with a type of a medical device 712 associated with the identifier element 714 and/or uniquely identify the medical device 712 associated with the identifier element 714 from other medical devices and/or indicate an orientation of the medical device 712 within environment 700 and/or with respect to another medical device 712 (e.g., a fluid flow path direction through a medical device 712, an input or inlet position and an output or outlet position of a medical device, etc.). For example, an identifier element 714 may encapsulate an identifier associated with at least one of the following types of medical devices: a peripheral IV catheter (PIVC), a peripherally inserted central catheter (PICC), a midline catheter, a needleless connector, a disinfectant cap, a disinfectant swab or wipe, an IV tubing set, an infusion pump, a flush syringe, a medication delivery syringe, a caregiver glove, an IV fluid bag, a medication dispensing cabinet, an ultrasound device, a sharps collector, or any combination thereof, and/or uniquely identify a medical device 712 from other medical devices including identifiers associated with a same type of medical device. In such an example, an identifier element 714 may include at least one of the following: a colored pattern, a reflective pattern, a fluorescent pattern, a predetermined shape and color, a LED pattern, a barcode, or any combination thereof, which may encapsulate the identifier.

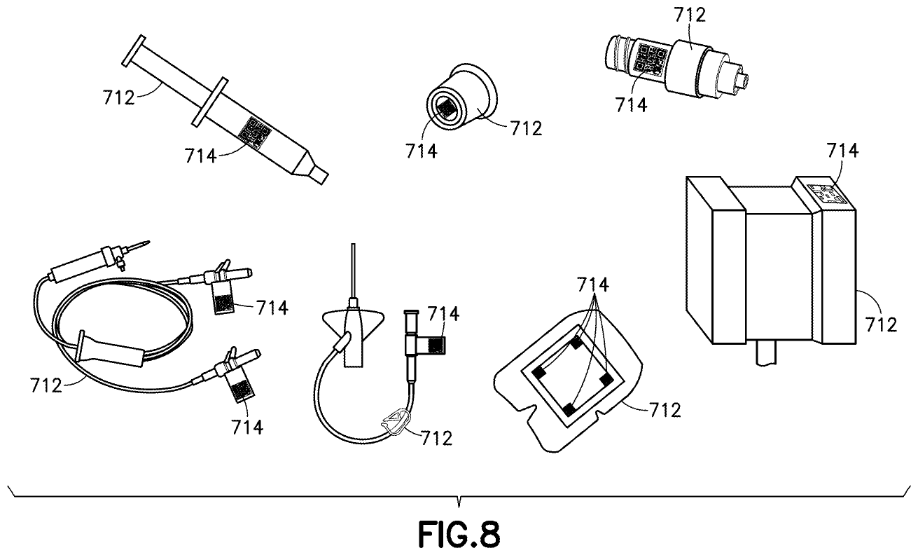

In some non-limiting embodiments or aspects, an identifier element 714 may include one or more colored areas, one or more reflective areas, one or more fluorescent areas, or any combination thereof that encapsulate an identifier. For example, an identifier element 714 may include one or more high-reflection areas such as, mirror surface particles, corner or edge reflectors, and/or the like, that encapsulate an identifier and render the identifier element 714 brighter than ambient illumination in environment 700. In such an example, an identifier element 714 may include a fluorescent coating or pattern on a medical device 712 that encapsulates an identifier by emitting light of a predetermined wavelength detectable in infrared by an image capture device including a filter configured to filter non-infrared light. In such an example, an identifier element 714 may include a tag or label having a predetermined shape and/or a predetermined color and/or color pattern that encapsulates an identifier (e.g., a green tag in a shape of a star, a red tag in a shape of a square, etc.). For example, an identifier element 714 may include a unique geometry and/or shape to differentiate itself from other identifier elements 714, and/or bars that wrap around cylindrical objects, grids, and/or patterns of shapes may be included in identifier element 714 for further identification and differentiation from other identifier elements 714. FIG. 9 is a perspective view of non-limiting embodiments or aspects of implementations of identifier elements 714 including color tags having a predetermined shape (e.g., a rectangle 0.5 inches by 1 inches, etc.) and/or a predetermined color (e.g., a first color, red, etc.) and/or color pattern (e.g., a first color and a second color in a pattern, red and blue in a pattern, etc.), fluorescent and/or reflective tags, and/or bar codes.

In some non-limiting embodiments or aspects, an identifier element 714 may include colors selected (e.g., optimized, etc.) to be detected by image capture system 702. For example, for image capture system 702 may include an RGB camera, and an identifier element 714 may include variable color regions to create unique tag identities. As an example, an individual color used in a variable color region may be created from a percentage (e.g., 0%, 50% or 100%) of one of R, G, and B, and R, G, and B can be used to create 33 or 27 color combinations of variable color regions for reliable differentiation of color. In such an example, multiple variable colors can be placed adjacent to one another to create even more combinations, such as a 2×2 grid of colors, 3 parallel bars of color, and/or the like.

In some non-limiting embodiments or aspects, an identifier element 714 may include color calibration areas positioned adjacent to variable color regions to calibrate color in a wider range of lighting conditions. For example, for a 2×2 grid, a cell (1,1) in an upper-left corner of the gird may include a predetermined and/or standard calibration color region (e.g., neutral gray, etc.), and image capture system 702 (e.g., sensor system 206, etc.), management system 102, and/or central computing system 202 may use the predetermined and/or standard calibration color region to calibrate colors in images used to detect or determine the identifier element 714 in those images. In such an example, image capture system 702 (e.g., sensor system 206, etc.), management system 102, and/or central computing system 202_may use the predetermined and/or standard calibration color region to orient identifier element 714 to determine how to properly rotate and decode the colors in identifier element 714 to decode the identifier encapsulated by the identifier element 714 and/or track the identifier element 714 within environment 700.

Accordingly, non-limiting embodiments or aspects of the present disclosure may use unique tags that identify medical devices individually and/or as a category or type of medical device for more robust image segmentation input, which may avoid the use of more standard bar code technology that may be more difficult to resolve without higher-cost cameras by instead using lower spatial resolution images, and lower cost cameras and processing. Further, in some non-limiting embodiments or aspects, additionally or alternatively to variable identifier elements 714 that identify product categories (e.g., by SKU, etc.), identifier elements 714 may include a hyper-variable region in which random hyper-variable tags may be applied during manufacturing. For example, if there are a predetermined number of unique identifiers (e.g., one hundred unique random identifiers, etc.), for any given patient, medical devices 712 may be uniquely identified, even if the medical devices 712 have the same SKU.

In some non-limiting embodiments or aspects, an identifier element 714 may include at least one light emitting diode (LED) (e.g., an RGB LED, an IR LED, etc.) configured to emit light of at least one predetermined wavelength in at least one predetermined pattern (e.g., a color code, a dynamic pattern, etc.) and/or at least one predetermined intensity, which encapsulates an identifier. For example, an identifier element 714 may include a battery (e.g., a rechargeable battery, a single use battery, a replaceable battery, etc.), an energy harvester (e.g., a thermoelectric energy harvester, a photovoltaic energy harvester, a piezoelectric energy harvester, etc.), a wireless power receiver (e.g., an RFID device, etc.), or any combination thereof that is configured to power the at least one LED and/or a controller configured to control the at least one LED to emit the light encapsulating the identifier, and image capture system 702 (e.g., sensor system 206, etc.), management system 102, and/or central computing system 202 may analyze the light captured in images to decode the identifier encapsulated by the identifier element 714 and/or track the identifier element 714 within environment 700.

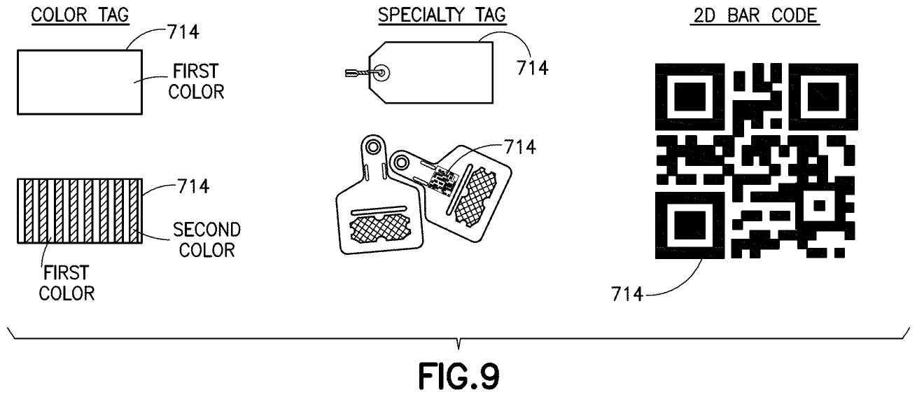

In some non-limiting embodiments or aspects, an identifier element 714 may include a 1D barcode and/or a 2D barcode (e.g., a QR code, an Aztec code, a Data Matrix code, an ArUco marker, etc.) that encapsulates an identifier. For example, as described in more detail herein below, in some non-limiting embodiments or aspects, image capture system 702 (e.g., sensor system 206, etc.), management system 102, and/or central computing system 202 may detect and/or track an identifier element 714 within environment 700 by detecting three square boxes on QR codes to reposition or orient an image and read a pattern in the QR code to identify a type of medical device 712 associated with the identifier element 714. For example, FIG. 10 illustrates non-limiting embodiments or aspects of implementations of identifier elements 714 including an ArUco marker, an Aztec code, and a Data Matrix code.

In some non-limiting embodiments or aspects, an identifier element 714 may include at least one color changing dye configured to change color over a period of time. For example, as described in more detail herein below, in some non-limiting embodiments or aspects, image capture system 702 (e.g., sensor system 206, etc.), management system 102, and/or central computing system 202 may determine, based on a change in the color of the color changing dye in a plurality of images, an amount of time associated with a use of a medical device 712 associated with the identifier element 714 (e.g., an amount of time since the medical device 712 is removed from a package, an amount of time medical device 712 is in environment 700, etc.).

Image capture system 702 (e.g., a camera system, a sensor system, sensor system 206, etc.) may include one or more image capture devices (e.g., one or more cameras, one or more sensors, etc.) configured to capture, over a period of time, a plurality of images (e.g., image data, etc.) of an environment (e.g., environment 700, an environment of a local system 104, etc.) surrounding the one or more image capture devices. For example, an image capture device may include at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

In some non-limiting embodiments or aspects, image capture system 702 may include a single camera configured to detect or capture only identifier elements 714 (e.g., from background, from other objects in environment 700, etc.). In some non-limiting embodiments or aspects, image capture system 702 may include a plurality of cameras configured to generate images with depth and/or to capture images from multiple different angles or fields-of-view to resolve occlusion of an object in a field of view of a single camera. In some non-limiting embodiments or aspects, image capture system 702 may include an IR camera configured to capture and/or read identifier elements 714 including infrared and/or near-infrared fluorescent tags or markings. In some non-limiting embodiments or aspects, image capture system 702 may include a PTZ camera configured to use variable FOV and automatic zoom functions to automatically zoom in on and capture zoomed images of medical devices 712 and/or identifier elements 714 that are identified by image capture system 702 as objects for which a more detailed image is to be captured by the PTZ camera (e.g., identified as objects likely to be a medical device 712 and/or an identifier element 714, etc.). In some non-limiting embodiments or aspects, image capture system 702 may include a master and slave camera system including a static camera configured to capture an initial image(s) and a dynamic camera configured to zoom in on and capture zoomed images of medical devices 712 and/or identifier elements 714 that are identified by image capture system 702, based on the images from the static camera, as objects for which a more detailed image is to be captured (e.g., identified as objects likely to be a medical device 712 and/or an identifier element 714, etc.). In some non-limiting embodiments or aspects, image capture system 702 may include a color camera configured to capture and/or detect one or more predetermined wavelengths of light. In some non-limiting embodiments or aspects, image capture system 702 may include a camera including a filter configured to filter a predetermined wavelength of light to distinguish medical devices 712 and/or identifier elements 714 from a background or scene based on a color of the medical devices 712 and/or identifier elements 714 in the captured images.

Image capture system 702 (e.g., sensor system 206, etc.), management system 102, and/or central computing system 202 may be configured to obtain image data and process the image data to determine object data associated with objects detected and/or determined from the image data. For example, image capture system 702 (e.g., sensor system 206, etc.), management system 102, and/or central computing system 202 may obtain image data from image capture system 702. As an example, image capture system 702 (e.g., sensor system 206, etc.), management system 102, and/or central computing system 202 may obtain a plurality of images, captured over a period of time, of an environment (e.g., environment 700, an environment of a local system 104, etc.) surrounding one or more image capture devices of image capture system 702. In such an example, image capture system 702 (e.g., sensor system 206, etc.), management system 102, and/or central computing system 202 may be configured to detect and/or determine, based on the images captured by image capture system 702 (e.g., based on image data, etc.), object data associated with at least one of the following: objects in the images (e.g., medical devices 712, identifier elements 714, medical devices 712 associated with identifier elements 714, etc.), types of the objects in the images, locations of the objects within environment 700 and/or with respect to other objects (e.g., other medical devices 712, other identifier elements 714, a patient, a caretaker, an image capture device, etc.), orientations (e.g., fluid flow path orientations through medical devices 712, inputs and outputs of medical devices 712, etc.) of the objects within environment 700 and/or with respect to the other objects, movements and/or trajectories of motion of the objects within environment 700 and/or with respect to the other objects, or any combination thereof. For example, image capture system 702 (e.g., sensor system 206, etc.), management system 102, and/or central computing system 202 may determine, based on the detected and/or determined objects in the images, object data associated with at least one of the following: the types of the objects in the images, the locations of the objects within environment 700 and/or with respect to other objects, the orientations of the objects within environment 700 and/or with respect to the other objects, and/or the movements and/or trajectories of motion of the objects within environment 700 and/or with respect to the other objects, or any combination thereof.

In some non-limiting embodiments or aspects, image capture system 702 (e.g., sensor system 206, etc.), management system 102, and/or central computing system 202 may differentiate medical devices 712 and/or identifier elements 714 from background and/or other objects in captured images based on object features detected and/or determined in the images, such as geometries of the medical devices 712 and/or the identifier elements 714, orientations in camera field-of-view of the medical devices 712 and/or the identifier elements 714, colors of the medical devices 712 and/or the identifier elements 714, proximities of the medical devices 712 and/or the identifier elements 714 to other medical devices 712 and/or other identifier elements 714, a patient, a caretaker, an image capture device, and/or the like, and/or interactive associations with the other medical devices 712 and/or the other identifier elements 714, the patient, the caretaker, the image capture device, and/or the like. For example, management system 102 may automatically document usage of medical devices 712 as the medical devices 712 and/or identifier elements 714 associated with the medical devices 712 are tracked within a field of view of the one or more image capture devices of image capture system 702 to provide event and/or usage-based guidance and alerts to caretakers, which may reduce complications during vascular access management assessment by continuously monitoring and updating usage information associated with the medical devices 712 and reducing errors associated with manual documentation.

In some non-limiting embodiments or aspects, image capture system 702 (e.g., sensor system 206, etc.), management system 102, and/or central computing system 202 may process the image data using one or more object detection techniques (e.g., a deep learning technique, an image processing technique, an image segmentation technique, etc.) to identify or determine medical devices 712 and/or identifier elements 714 in the images of the image data and/or in the object data associated with the medical devices 712 and/or the identifier elements 714. For example, a deep learning technique may include a bounding box technique that generates a box label for objects (e.g., medical devices 712, identifier elements 714, etc.) of interest in images, an image masking technique (e.g., masked FRCNN (RCNN or CNN) that captures specific shapes of objects (e.g., medical devices 712, identifier elements 714, etc.) in images, a trained neural network that identifies objects (e.g., medical devices 712, identifier elements 714, etc.) in images, and/or the like. As an example, an image processing technique may include a cross correlation image processing technique, an image contrasting technique, a binary or colored filtering technique, and/or the like.

In some non-limiting embodiments or aspects, image capture system 702 (e.g., sensor system 206, etc.), management system 102, and/or central computing system 202 may process the image data using a stereoscopic imaging technique and/or a shadow distance technique to determine object data including a distance from image capture system 702 to detected objects and/or distances between detected objects, and/or image capture system 702 may obtain the image data using multiple cameras, a laser focus technology, LiDAR sensors, and/or a camera physical zoom-in function to determine object data including a distance from image capture system 702 to detected objects and/or distances between detected objects. In some non-limiting embodiments or aspects, image capture system 702 may obtain image data and/or object data including a 3D profile of an object using a 3D optical profiler.

In some non-limiting embodiments or aspects, image capture system 702 (e.g., sensor system 206, etc.), management system 102, and/or central computing system 202 may determine, based on image data and/or object data, event data associated with events and/or activities (e.g., vascular access management events, connections between medical devices 712, disconnections of medical devices 712, uses of medical devices, such as scrubbing events, disinfecting events, reuses of medical devices 712, replacements of medical devices 712, and/or the like, etc.) associated with the detected objects and/or an amount of time associated with the determined events and/or activities (e.g., an amount of time medical devices 712 are connected, a scrubbing time associated with a medical device 712, a drying time associated with a medical device after the scrubbing time, etc.).

In some non-limiting embodiments or aspects, image capture system 702 (e.g., sensor system 206, etc.), management system 102, and/or central computing system 202 may generate, based on image data and/or object data (e.g., based on locations of detected medical devices 712 and/or identifier elements 714, types of the detected medical devices 712 and/or identifier elements 714, orientations of the detected medical devices 712 and/or identifier elements 714 relative to one another, movements and/or trajectories of the detected medical devices 712 and/or identifier elements 714 relative to one another, etc.), event data including a relational model of which medical devices 712 are connected to one another, as well as determine when these medical devices 712 are connected to each other and/or a duration of connection, disconnected from each other and/or a duration of disconnection, are involved in one or more other events or caretaker activities, and/or times associated therewith. For example, image capture system 702 (e.g., sensor system 206, etc.), management system 102, and/or central computing system 202 may use one or more criteria, such as a threshold distance between points of medical devices 712, relative orientations and/or direction vectors of the medical devices 712, threshold times associated therewith, and/or the like, to determine event data associated with whether medical devices 712 are connected and/or disconnected from each other (e.g., whether a fluid path connection is established between medical devices 712, etc.), whether another event involving one or more of the medical devices 712 has occurred (e.g., a scrubbing or disinfecting event, etc.), and/or times associated therewith. In such an example, as medical devices 712 are moved in environment 700, continuing connections of the same medical devices 712 at or over different points in time (and/or in different images) may increase a probability that those medical devices are connected in a fluid pathway.

In some non-limiting embodiments or aspects, image capture system 702 (e.g., sensor system 206, etc.), management system 102, and/or central computing system 202 may generate one or more models (e.g., an estimator, a classifier, a prediction model, a detector model, etc.) using one or more machine learning techniques including, for example, supervised and/or unsupervised techniques, such as decision trees (e.g., gradient boosted decision trees, random forests, etc.), logistic regressions, artificial neural networks (e.g., convolutional neural networks, etc.), Bayesian statistics, learning automata, Hidden Markov Modeling, linear classifiers, quadratic classifiers, association rule learning, and/or the like. Management system 104 may generate the model based on image data and/or object data (e.g., training data, etc.) associated with one or more environments. In some implementations, the model is designed to receive, as an input, image data and/or object data and provide, as an output, a prediction (e.g., a probability, a binary output, a yes-no output, a score, a prediction score, a classification, event data, etc.) as to whether one or more events have occurred. In some non-limiting embodiments, image capture system 702 (e.g., sensor system 206, etc.), management system 102, and/or central computing system 202 stores the model (e.g., stores the model for later use). In some non-limiting embodiments or aspects, image capture system 702 (e.g., sensor system 206, etc.), management system 102, and/or central computing system 202 may store the model in a data structure (e.g., a database, a linked list, a tree, etc.). In some non-limiting embodiments, the data structure is located within image capture system 702 (e.g., sensor system 206, etc.), management system 102, and/or central computing system 202 or external (e.g., remote from) image capture system 702 (e.g., sensor system 206, etc.), management system 102, and/or central computing system 202. The one or more machine learning models may be trained to provide an output including event data associated with a prediction or classification of an event or activity associated with one or more medical devices 712 in response to input including image data and/or object data. In such an example, the prediction or classification of an event may include at least one of the following predictions or classifications: (i) a reuse of a medical device including a disconnection of the medical device from at least one of a patient and another medical device a reconnection of the medical device to the at least one of the patient and the another medical device, (ii) a replacement of a medical device with a new medical device of a same type as the medical device (e.g., a catheter dressing change, etc.) (iii) a connection of a first medical device to a second medical device, (iv) a disconnection of a first medical device from a second medical device, (v) a scrubbing or disinfecting event including scrubbing or disinfecting a medical device with another medical device, (vi) a drying event including an amount of time a medical device remains disconnected from other medical devices after a scrubbing or disinfecting event, or any combination thereof. In some non-limiting embodiments or aspects, a prediction or classification may include a probability score associated with a class prediction for an event. For example, the prediction or classification of the event may include a probability that the event occurred. As an example, the prediction or classification of the event may include at least one of the following: (i) a probability that a reuse of a medical device occurred (e.g., a reuse of a disinfectant swab or wipe, etc.), (ii) a probability that a replacement of a medical device occurred (e.g., a catheter dressing change, etc.) (iii) a probability that a connection of a first medical device to a second medical device occurred, (iv) a probability that a disconnection of a first medical device from a second medical device occurred, (v) a probability that a scrubbing or disinfecting event occurred, (vi) a probability that a drying event occurred, or any combination thereof.

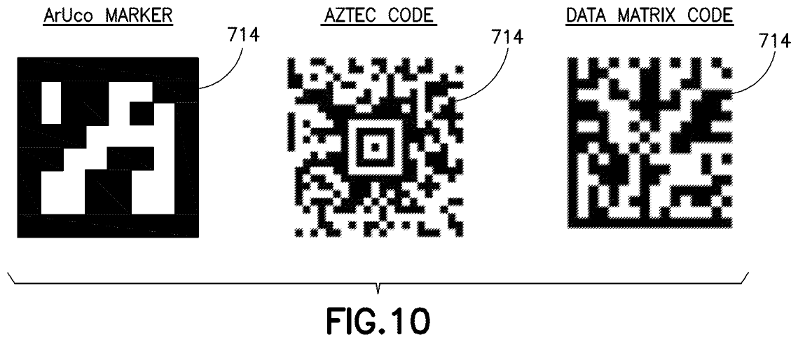

In some non-limiting embodiments or aspects, image capture system 702 (e.g., sensor system 206, etc.), management system 102, and/or central computing system 202 may generate and/or update, based on the image data, the object data, and/or the event data, a database including locations of detected medical devices 712 and/or identifier elements 714, types of the detected medical devices 712 and/or identifier elements 714, orientations of the detected medical devices 712 and/or identifier elements 714 relative to one another, movements and/or trajectories of the detected medical devices 712 and/or identifier elements 714 relative to one another, which medical devices 712 are connected to one another, when medical devices 712 are connected to each other and/or a duration of connection, which medical devices 712 are disconnected from each other and/or a duration of disconnection, which medical devices 712 are involved in one or more other events or activities and/or a duration thereof, and/or times associated therewith. For example, image capture system 702 (e.g., sensor system 206, etc.), management system 102, and/or central computing system 202 may maintain and update a database that includes spatial relationships of medical devices 712 and/or identifier elements 714 (e.g., distances between medical devices 712 and/or identifier elements 714, etc.) at a gross level (e.g., where an object is represented as a point, etc.) and orientations of the objects (e.g., fluid path vectors associated with a fluid path direction through a medical device, etc.), 3-space data that includes point locations of input-connectors and output connectors of medical devices 712, as well as intermediate points for larger devices, and/or the like, and/or determined events or activities therebetween. As an example, the database may include at least one of the following: a list of the plurality of medical devices currently in the environment, spatial relationships between the plurality of medical devices, current connections between the plurality of medical devices, current trajectories of the plurality of medical devices, orientations of the plurality of medical devices, events associated with the plurality of medical devices, or any combination thereof. In such an example, image capture system 702 (e.g., sensor system 206, etc.), management system 102, and/or central computing system 202 may control a display (e.g., a display of user device 208, etc.) to display a visual representation of the information stored and/or maintained in the database. For example, FIG. 11 illustrates an example visual representation 1100 of the implementation of environment 700 shown in FIG. 7. As shown in FIG. 11, image capture system 702 (e.g., sensor system 206, etc.), management system 102, and/or central computing system 202 may represent detected medical devices 712 and/or identifier elements 714 may be as points associated with identifiers and spatial distances between the medical devices 712 and/or identifier elements 714.

For example, as shown in FIG. 11, user device 208 may be configured to display spatial relationships of medical devices 712 and/or identifier elements 714 with the medical devices 712 and/or identifier elements 714 represented as points in the display indicating the locations or most probable locations of the medical devices 712 and/or identifier elements 714 determined by image capture system 702 (e.g., sensor system 206, etc.), management system 102, and/or central computing system 202, indications of connections between the medical devices 712 and/or identifier elements 714 represented as points, and/or indication of orientations of the medical devices 712 and/or identifier elements 714 represented as points. As an example, user device 208 may be configured to automatically provide, in response to a determination of an event associated with one or more medical devices, an alert associated with the determined event (e.g., display instructions or a warning associated with event, emit audible instructions or warnings associated with the event, etc.).

Referring now to FIG. 12, FIG. 12 is a flowchart of non-limiting embodiments or aspects of a process 1200 for obtaining VAM data. In some non-limiting embodiments or aspects, one or more of the steps of process 1200 may be performed (e.g., completely, partially, etc.) by management system 102 (e.g., one or more devices of management system 102, etc.). In some non-limiting embodiments or aspects, one or more of the steps of process 1200 may be performed (e.g., completely, partially, etc.) by another device or a group of devices separate from or including management system 102, such as local system 104 (e.g., one or more devices of local system 104, etc.), central computing system 202 (e.g., one or more devices of central computing system 202, etc.), medication source system 204 (e.g., one or more devices of medication source system 204, etc.), sensor system 206 (e.g., one or more devices of sensor system 206, etc.), and/or user device 208 (e.g., one or more devices of a system of user device 208, etc.).

As shown in FIG. 12, at step 1202, process 1200 includes obtaining images. For example, image capture system 702 (e.g., sensor system 206, etc.), management system 102, and/or central computing system 202 may obtain images. As an example, one or more image capture devices of image capture system 702 may capture, over a period of time, a plurality of images of an environment (e.g., environment 700, etc.) surrounding the one or more image capture devices. In such an example, management system 102 and/or central computing system 202 may obtain the plurality of images from image capture system 702 (e.g., sensor system 206, etc.).

As shown in FIG. 12, at step 1204, process 1200 includes determining locations, types, trajectories, and/or orientations of medical devices. For example, image capture system 702 (e.g., sensor system 206, etc.), management system 102, and/or central computing system 202 may determine, based on the plurality of images, a plurality of locations of a plurality of medical devices 712 within the environment over the period of time, a plurality of types of the plurality of medical devices 712, a plurality of trajectories of the plurality of medical devices 712 within the environment over the period of time, and/or a plurality of orientations of the plurality of medical devices 712 within the environment over the period of time.

As shown in FIG. 12, at step 1206, process 1200 includes determining at least one event associated with at least one medical device. For example, image capture system 702 (e.g., sensor system 206, etc.), management system 102, and/or central computing system 202 may determine, based on the plurality of locations of the plurality of medical devices 712 within the environment over the period of time, the plurality of types of the plurality of medical devices 712, the plurality of trajectories of the plurality of medical devices 712 within the environment over the period of time, and/or the plurality of orientations of the plurality of medical devices 712 within the environment over the period of time, at least one event associated with at least one medical device of the plurality of medical devices 712.

In some non-limiting embodiments or aspects, the at least one event may include a connection between two or more medical devices of the plurality of medical devices 712. For example, image capture system 702 (e.g., sensor system 206, etc.), management system 102, and/or central computing system 202 may determine, based on the plurality of locations of the plurality of medical devices 712 within the environment over the period of time, the plurality of types of the plurality of medical devices 712, the plurality of trajectories of the plurality of medical devices 712 within the environment over the period of time, and/or the plurality of orientations of the plurality of medical devices 712 within the environment over the period of time, at least one connection between two or more medical devices of the plurality of medical devices 712.

In some non-limiting embodiments or aspects, at least one connection between two or more medical devices forms a fluid flow path through the two or more medical devices. For example, image capture system 702 (e.g., sensor system 206, etc.), management system 102, and/or central computing system 202 may determine, based on the plurality of locations of the plurality of medical devices 712 within the environment over the period of time, the plurality of types of the plurality of medical devices 712, the plurality of trajectories of the plurality of medical devices 712 within the environment over the period of time, and/or the plurality of orientations of the plurality of medical devices 712 within the environment over the period of time, at least one event including at least one connection between two or more medical devices of the plurality of medical devices 712 that forms a fluid flow path through the two or more medical devices and a direction of a fluid flow in the fluid flow path through the two or more medical devices.

In some non-limiting embodiments or aspects, the at least one event may include at least one event of the following events: (i) a reuse of a medical device including a disconnection of the medical device from at least one of a patient and another medical device in the environment and a reconnection of the medical device to the at least one of the patient and the another medical device in the environment and (ii) a replacement of the medical device with a new medical device of a same type as the medical device in the environment. For example, image capture system 702 (e.g., sensor system 206, etc.), management system 102, and/or central computing system 202 may determine, based on the plurality of locations of the plurality of medical devices 712 within the environment over the period of time, the plurality of types of the plurality of medical devices 712, the plurality of trajectories of the plurality of medical devices 712 within the environment over the period of time, and/or the plurality of orientations of the plurality of medical devices 712 within the environment over the period of time, at least one event of the following events: (i) a reuse of a medical device including a disconnection of the medical device from at least one of a patient and another medical device in the environment and a reconnection of the medical device to the at least one of the patient and the another medical device in the environment and (ii) a replacement of the medical device with a new medical device of a same type as the medical device in the environment.

As shown in FIG. 12, at step 1208, process 1200 includes providing VAM data associated with the at least one event (e.g., event data, etc.). For example, image capture system 702 (e.g., sensor system 206, etc.), management system 102, and/or central computing system 202 may update a database, provide an alert, and/or control at least one medical device based on the at least one event associated with the at least one medical device, the plurality of locations of the plurality of medical devices 712 within the environment over the period of time, the plurality of types of the plurality of medical devices 712, the plurality of trajectories of the plurality of medical devices 712 within the environment over the period of time, and/or the plurality of orientations of the plurality of medical devices 712 within the environment over the period of time.

In some non-limiting embodiments or aspects, in response to the at least one event including a reuse of a medical device, image capture system 702 (e.g., sensor system 206, etc.), management system 102, and/or central computing system 202 may at least one of: provide to user device 208, an alert associated with the use of the medical device 712; and automatically control at least one medical device (e.g., a valve, an infusion pump, etc.) to stop a flow of fluid in a fluid flow path including the medical device.

Referring now to FIG. 13, FIG. 13 is a flowchart of non-limiting embodiments or aspects of a process 1300 for obtaining VAM data. In some non-limiting embodiments or aspects, one or more of the steps of process 1300 may be performed (e.g., completely, partially, etc.) by management system 102 (e.g., one or more devices of management system 102, etc.). In some non-limiting embodiments or aspects, one or more of the steps of process 1300 may be performed (e.g., completely, partially, etc.) by another device or a group of devices separate from or including management system 102, such as local system 104 (e.g., one or more devices of local system 104, etc.), central computing system 202 (e.g., one or more devices of central computing system 202, etc.), medication source system 204 (e.g., one or more devices of medication source system 204, etc.), sensor system 206 (e.g., one or more devices of sensor system 206, etc.), and/or user device 208 (e.g., one or more devices of a system of user device 208, etc.).

As shown in FIG. 13, at step 1302, process 1300 includes obtaining images. For example, image capture system 702 (e.g., sensor system 206, etc.), management system 102, and/or central computing system 202 may obtain images. As an example, one or more image capture devices of image capture system 702 may capture, over a period of time, a plurality of images of an environment (e.g., environment 700, etc.) surrounding the one or more image capture devices. In such an example, management system 102 and/or central computing system 202 may obtain the plurality of images from image capture system 702.

As shown in FIG. 13, at step 1304, process 1300 includes determining identifier elements in images. For example, image capture system 702 (e.g., sensor system 206, etc.), management system 102, and/or central computing system 202 may determine, based on the plurality of images, a plurality of identifier elements 714 within the environment over the period of time. As an example, the plurality of identifier elements 714 may be associated with a plurality of medical devices 712, and the plurality of identifier elements 714 may encapsulate a plurality of identifiers associated a plurality of types of the plurality of medical devices 712. In some non-limiting embodiments or aspects, the plurality of identifiers may uniquely identify the plurality of medical devices 712 from each other.

In some non-limiting embodiments or aspects, the plurality of identifier elements 714 may include at least one identifier element including a fluorescent coating configured to emit light of a predetermined wavelength, and image capture system 702 may capture only the light of the predetermined wavelength in the plurality of images.

In some non-limiting embodiments or aspects, the plurality of identifier elements 714 includes at least one identifier element including at least one LED configured to emit light of at least one predetermined wavelength in at least one pattern and/or at least one intensity, and image capture system 702 may capture only the light of the predetermined wavelength in the plurality of images. For example, image capture system 702 (e.g., sensor system 206, etc.), management system 102, and/or central computing system 202 may determine, based on the at least one pattern and/or the at least one intensity of the emitted light of the at least one predetermined wavelength captured in the plurality of images, a type of a medical device associated with the at least one identifier element.

In some non-limiting embodiments or aspects, the plurality of identifier elements 714 includes at least one identifier element including at least one color changing dye configured to change color over the period of time. For example, image capture system 702 (e.g., sensor system 206, etc.), management system 102, and/or central computing system 202 may determine, based on a change in the color of the color changing dye in the plurality of images, an amount of time associated with a use of a medical device associated with the at least one identifier element.

As shown in FIG. 13, at step 1306, process 1300 includes determining locations, types, trajectories, and/or orientations of medical devices. For example, image capture system 702 (e.g., sensor system 206, etc.), management system 102, and/or central computing system 202 may determine, based on the plurality of identifier elements determined in the plurality of images, a plurality of locations of the plurality of medical devices 712 within the environment over the period of time, a plurality of types of the plurality of medical devices 712, a plurality of trajectories of the plurality of medical devices 712 within the environment over the period of time, and/or a plurality of orientations of the plurality of medical devices 712 within the environment over the period of time.

As shown in FIG. 13, at step 1308, process 1300 includes determining at least one event associated with at least one medical device. For example, image capture system 702 (e.g., sensor system 206, etc.), management system 102, and/or central computing system 202 may determine, based on the plurality of locations of the plurality of medical devices 712 within the environment over the period of time, the plurality of types of the plurality of medical devices 712, the plurality of trajectories of the plurality of medical devices 712 within the environment over the period of time, and/or the plurality of orientations of the plurality of medical devices 712 within the environment over the period of time, at least one event associated with at least one medical device of the plurality of medical devices 712.

As shown in FIG. 13, at step 1310, process 1300 includes obtaining VAM data associated with the at least one event (e.g., event data, etc.). For example, image capture system 702 (e.g., sensor system 206, etc.), management system 102, and/or central computing system 202 may update a database, provide an alert, and/or control at least one medical device based on the at least one event associated with the at least one medical device, the plurality of locations of the plurality of medical devices 712 within the environment over the period of time, the plurality of types of the plurality of medical devices 712, the plurality of trajectories of the plurality of medical devices 712 within the environment over the period of time, and/or the plurality of orientations of the plurality of medical devices 712 within the environment over the period of time.

In some non-limiting embodiments or aspects, in response to the at least one event including a reuse of a medical device, image capture system 702 (e.g., sensor system 206, etc.), management system 102, and/or central computing system 202 may at least one of: provide to user device 208, an alert associated with the use of the medical device 712; and automatically control at least one medical device (e.g., a valve, an infusion pump, etc.) to stop a flow of fluid in a fluid flow path including the medical device.

Referring now to FIG. 14, FIG. 14 is a flowchart of non-limiting embodiments or aspects of a process 1400 for obtaining VAM data. In some non-limiting embodiments or aspects, one or more of the steps of process 1400 may be performed (e.g., completely, partially, etc.) by management system 102 (e.g., one or more devices of management system 102, etc.). In some non-limiting embodiments or aspects, one or more of the steps of process 1400 may be performed (e.g., completely, partially, etc.) by another device or a group of devices separate from or including management system 102, such as local system 104 (e.g., one or more devices of local system 104, etc.), central computing system 202 (e.g., one or more devices of central computing system 202, etc.), medication source system 204 (e.g., one or more devices of medication source system 204, etc.), sensor system 206 (e.g., one or more devices of sensor system 206, etc.), and/or user device 208 (e.g., one or more devices of a system of user device 208, etc.).

As shown in FIG. 14, at step 1402, process 1400 includes obtaining images. For example, image capture system 702 (e.g., sensor system 206, etc.), management system 102, and/or central computing system 202 may obtain images. As an example, image capture system 702 may capture, over a period of time, a plurality of images of an environment (e.g., environment 700, etc.) surrounding the one or more image capture devices. In such an example, management system 102 and/or central computing system 202 may obtain the plurality of images from image capture system 702 (e.g., sensor system 206, etc.).

As shown in FIG. 14, at step 1404, process 1400 includes determining locations, types, trajectories, and/or orientations of medical devices. For example, image capture system 702 (e.g., sensor system 206, etc.), management system 102, and/or central computing system 202 may determine, based on the plurality of images, a plurality of locations of a plurality of medical devices 712 within the environment over the period of time, a plurality of types of the plurality of medical devices 712, a plurality of trajectories of the plurality of medical devices 712 within the environment over the period of time, and/or a plurality of orientations of the plurality of medical devices 712 within the environment over the period of time.

As shown in FIG. 14, at step 1406, process 1400 includes determining distances between medical devices. For example, image capture system 702 (e.g., sensor system 206, etc.), management system 102, and/or central computing system 202 may determine, based on the plurality of locations of the plurality of medical devices 712 within the environment over the period of time, the plurality of types of the plurality of medical devices 712, the plurality of trajectories of the plurality of medical devices 712 within the environment over the period of time, and/or the plurality of orientations of the plurality of medical devices 712 within the environment over the period of time, a plurality of distances between the plurality of medical devices 712 over the period of time.

In some non-limiting embodiments or aspects, image capture system 702 (e.g., sensor system 206, etc.), management system 102, and/or central computing system 202 may delay determining the plurality of distances between the plurality of medical devices 712 over the period of time and determining the at least one event until a location of at least one of the first medical device and the second medical device changes in the plurality of images over the period of time. Accordingly, image capture system 702 (e.g., sensor system 206, etc.), management system 102, and/or central computing system 202 may save processing power and/or other computer resources until they are needed to detect a change in environment 700.

As shown in FIG. 14, at step 1408, process 1400 includes determining at least one event associated with at least one medical device. For example, image capture system 702 (e.g., sensor system 206, etc.), management system 102, and/or central computing system 202 may determine, based on the plurality of distances between the plurality of medical devices 712 over the period of time and the plurality of types of the plurality of medical devices 712 (and/or the plurality of locations of the plurality of medical devices 712 within the environment over the period of time, the plurality of trajectories of the plurality of medical devices 712 within the environment over the period of time, and/or the plurality of orientations of the plurality of medical devices 712 within the environment over the period of time), at least one event associated with at least one medical device of the plurality of medical devices 712. As an example, image capture system 702 (e.g., sensor system 206, etc.), management system 102, and/or central computing system 202 may determine, based on the plurality of distances between the plurality of medical devices 712 over the period of time and the plurality of types of the plurality of medical devices 712, at least one event of the following events: (i) a connection of a first medical device of the plurality of medical devices 712 to a second medical device of the plurality of medical devices 712 and (ii) a disconnection of the first medical device of the plurality of medical devices 712 from the second medical device of the plurality of medical devices 712. In some non-limiting embodiments or aspects, determining the at least one event further determines a probability associated with the at least one event.

In some non-limiting embodiments or aspects, image capture system 702 (e.g., sensor system 206, etc.), management system 102, and/or central computing system 202 may determine based on the plurality of distances between the plurality of medical devices 712 over the period of time and the plurality of types of the plurality of medical devices 712, one of the following further events: (i) a reuse of the first medical device including a disconnection of the first medical device from the second medical device in the environment and a reconnection of the first medical device to the second medical device in the environment and (ii) a replacement of the first medical device with a new medical device of a same type as the first medical device.

In some non-limiting embodiments or aspects, image capture system 702 (e.g., sensor system 206, etc.), management system 102, and/or central computing system 202 may determine, based on an orientation of the first medical device and an orientation of the second medical device, a direction of a fluid flow in a fluid flow path through the first medical device and the second medical device and/or update, based on the direction of the fluid flow in the fluid flow path through the first medical device and the second medical device, a database.

In some non-limiting embodiments or aspects, the first medical device includes at least one of a disinfectant cap and a disinfectant swab such that the connection of the first medical device of the plurality of medical devices 712 to the second medical device of the plurality of medical devices 712 does not form a fluid flow path through the first medical device and the second medical device. For example, the connection of the first medical device of the plurality of medical devices 712 to the second medical device of the plurality of medical devices 712 may be associated with a scrubbing event including scrubbing of the second medical device (e.g., a needleless connector, etc.) with the disinfectant cap and/or the disinfectant swab.

As shown in FIG. 14, at step 1410, process 1400 includes obtaining VAM data associated with the at least one event (e.g., event data, etc.). For example, image capture system 702 (e.g., sensor system 206, etc.), management system 102, and/or central computing system 202 may update a database, provide an alert, and/or control at least one medical device based on the at least one event associated with the at least one medical device, the plurality of locations of the plurality of medical devices 712 within the environment over the period of time, the plurality of types of the plurality of medical devices 712, the plurality of trajectories of the plurality of medical devices 712 within the environment over the period of time, and/or the plurality of orientations of the plurality of medical devices 712 within the environment over the period of time.

In some non-limiting embodiments or aspects, in response to the at least one event including a reuse of a medical device, image capture system 702 (e.g., sensor system 206, etc.), management system 102, and/or central computing system 202 may at least one of: provide to user device 208, an alert associated with the use of the medical device 712; and automatically control at least one medical device (e.g., a valve, an infusion pump, etc.) to stop a flow of fluid in a fluid flow path including the medical device.

Referring now to FIG. 15, FIG. 15 is a flowchart of non-limiting embodiments or aspects of a process 1500 for obtaining VAM data. In some non-limiting embodiments or aspects, one or more of the steps of process 1500 may be performed (e.g., completely, partially, etc.) by management system 102 (e.g., one or more devices of management system 102, etc.). In some non-limiting embodiments or aspects, one or more of the steps of process 1500 may be performed (e.g., completely, partially, etc.) by another device or a group of devices separate from or including management system 102, such as local system 104 (e.g., one or more devices of local system 104, etc.), central computing system 202 (e.g., one or more devices of central computing system 202, etc.), medication source system 204 (e.g., one or more devices of medication source system 204, etc.), sensor system 206 (e.g., one or more devices of sensor system 206, etc.), and/or user device 208 (e.g., one or more devices of a system of user device 208, etc.).

As shown in FIG. 15, at step 1502, process 1500 includes obtaining images. For example, image capture system 702 (e.g., sensor system 206, etc.), management system 102, and/or central computing system 202 may obtain images. As an example, image capture system 702 may capture, over a period of time, a plurality of images of an environment (e.g., environment 700, etc.) surrounding the one or more image capture devices. In such an example, management system 102 and/or central computing system 202 may obtain the plurality of images from image capture system 702 (e.g., sensor system 206, etc.).

As shown in FIG. 15, at step 1504, process 1500 includes determining a first identifier element associated with a medical device and a second identifier element associated with a glove of a caregiver. For example, image capture system 702 (e.g., sensor system 206, etc.), management system 102, and/or central computing system 202 may determine, based on the plurality of images, a first identifier element associated with a medical device (e.g., a needleless connector, etc.) and a second identifier element associated with a glove of a caregiver. As an example, the first identifier element may encapsulate a first identifier associated with the medical device, and the second identifier element may encapsulate a second identifier associated with the glove of the caregiver.

As shown in FIG. 15, at step 1506, process 1500 includes determining locations, types, trajectories, and/or orientations of the medical device associated with the first identifier element. For example, image capture system 702 (e.g., sensor system 206, etc.), management system 102, and/or central computing system 202 may determine, based on the first identifier element in the plurality of images, the medical device and a location, a type, a trajectory, and/or an orientation of the medical device within the environment over the period of time.

As shown in FIG. 15, at step 1508, process 1500 includes determining locations, types, trajectories, and/or orientations of the caregiver glove associated with the second identifier element. For example, image capture system 702 (e.g., sensor system 206, etc.), management system 102, and/or central computing system 202 may determine, based on the second identifier element in the plurality of images, the glove of the caregiver and a location, a type, a trajectory, and/or an orientation of the glove of the caregiver within the environment over the period of time. As an example, the second identifier may include a predetermined color of the glove of the caregiver.

As shown in FIG. 15, at step 1510, process 1500 includes determining at least one event associated with the medical device and the caregiver glove. For example, image capture system 702 (e.g., sensor system 206, etc.), management system 102, and/or central computing system 202 may determine, based on the location, the type, the trajectory, and/or the orientation of the medical device within the environment over the period of time and the location, the type, the trajectory, and/or the orientation of the glove of the caregiver within the environment over the period of time and the location, at least one event associated with the medical device. In such an example, the at least one event may include a catheter dressing change event including replacing the medical device (e.g., a catheter dressing, etc.) with a new medical device of the same type.

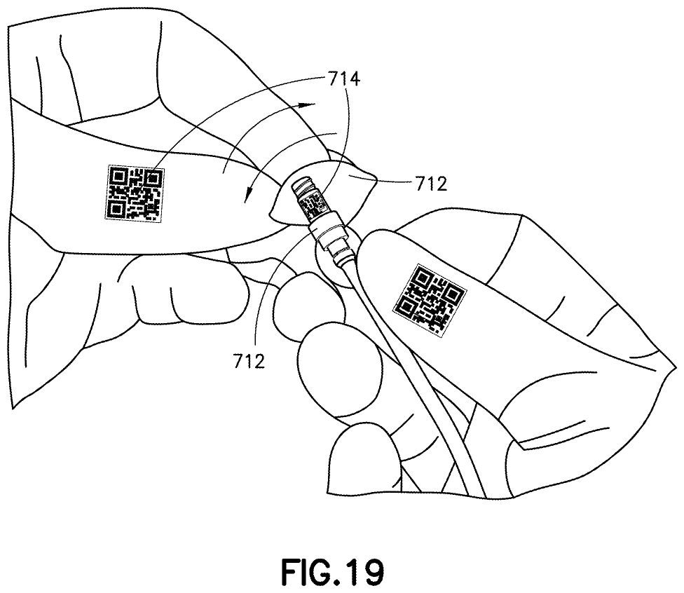

In some non-limiting embodiments or aspects, and referring also to FIG. 19, image capture system 702 (e.g., sensor system 206, etc.), management system 102, and/or central computing system 202 may determine, based on the plurality of images, a location, a type, a trajectory, and/or an orientation of a further medical device (e.g., a disinfectant wipe, etc.) within the environment over the period of time. For example, image capture system 702 (e.g., sensor system 206, etc.), management system 102, and/or central computing system 202 may determine, based on the location, the type, the trajectory, and/or the orientation of the further medical device over the period of time and at least one of (i) the location, the type, the trajectory, and/or the orientation of medical device within the environment over the period of time and (ii) the location, the type, the trajectory, and/or the orientation of the glove of the caregiver within the environment over the period of time, a linear distance change and an angular distance change between the further medical device and the at least one of the medical device and the glove of the caregiver over the period of time. As an example, image capture system 702 (e.g., sensor system 206, etc.), management system 102, and/or central computing system 202 may further determine the at least one event and/or a duration of the at least one event based on the linear distance change and the angular distance change between the medical device and the glove of the caregiver. In such an example, as shown in FIG. 19, the at least one event may include a scrubbing or disinfecting event including scrubbing of the medical device (e.g., a needleless connector etc.) held in the glove of the caregiver with the further medical device (e.g., a disinfectant swab or wipe, etc.) held in the other glove of the caregiver.

As shown in FIG. 15, at step 1512, process 1500 includes obtaining VAM data associated with the at least one determined event (e.g., event data, etc.). For example, management system 102 may, based on the at least one determined event, update a database including events associated with the environment, provide an alert associated with the at least one determined event, and/or control one or more medical devices in the environment.

In some non-limiting embodiments or aspects, in response to the at least one event, image capture system 702 (e.g., sensor system 206, etc.), management system 102, and/or central computing system 202 may at least one of: provide to user device 208, an alert associated with the at least one event; and automatically control at least one medical device (e.g., a valve, an infusion pump, etc.) to stop a flow of fluid in a fluid flow path including the medical device associated with the at least one event.

Referring now to FIGS. 16A and 16B, FIGS. 16A and 16B are a flowchart of non-limiting embodiments or aspects of a process 1600 for obtaining VAM data. In some non-limiting embodiments or aspects, one or more of the steps of process 1600 may be performed (e.g., completely, partially, etc.) by management system 102 (e.g., one or more devices of management system 102, etc.). In some non-limiting embodiments or aspects, one or more of the steps of process 1600 may be performed (e.g., completely, partially, etc.) by another device or a group of devices separate from or including management system 102, such as local system 104 (e.g., one or more devices of local system 104, etc.), central computing system 202 (e.g., one or more devices of central computing system 202, etc.), medication source system 204 (e.g., one or more devices of medication source system 204, etc.), sensor system 206 (e.g., one or more devices of sensor system 206, etc.), and/or user device 208 (e.g., one or more devices of a system of user device 208, etc.).

As shown in FIG. 16A, at step 1602, process 1600 includes obtaining images. For example, image capture system 702 (e.g., sensor system 206, etc.), management system 102, and/or central computing system 202 may obtain images. As an example, image capture system 702 may capture, over a period of time, a plurality of images of an environment (e.g., environment 700, etc.) surrounding the one or more image capture devices. In such an example, management system 102 and/or central computing system 202 may obtain the plurality of images from image capture system 702 (e.g., sensor system 206, etc.).

As shown in FIG. 16A, at step 1604, process 1600 includes determining a location of a plunger of a syringe relative to a barrel of the syringe. For example, image capture system 702 (e.g., sensor system 206, etc.), management system 102, and/or central computing system 202 may determine, based on the plurality of images, a location of a plunger of a syringe relative to a barrel of the syringe in the environment over the period of time.

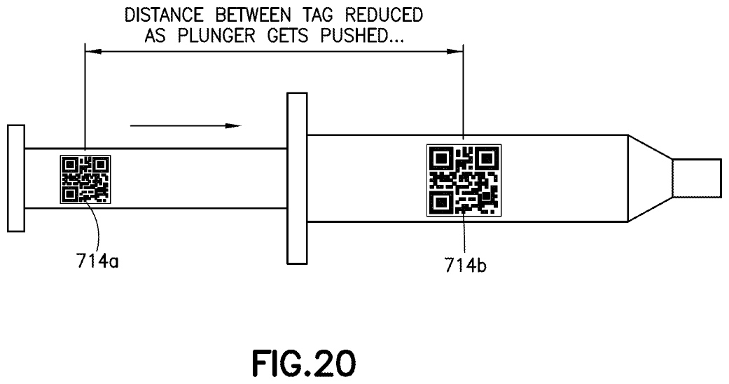

In some non-limiting embodiments or aspects, and referring also to FIG. 20, image capture system 702 (e.g., sensor system 206, etc.), management system 102, and/or central computing system 202 may determine the location of the plunger of the syringe relative to the barrel of the syringe over the period of time further by determining, based on the plurality of images, a first identifier element 714a associated with the plunger of the syringe and a second identifier element 714b associated with the barrel of the syringe, and the location of the plunger of a syringe relative to a barrel of the syringe in the environment over the period of time is determined based on the first identifier element 714a in the plurality of images and the second identifier element 714b in the plurality of images. For example, determining the location of the plunger of the syringe relative to the barrel of the syringe in the environment over the period of time may include determining, based on the first identifier element 714a and the second identifier element 714b in the plurality of images, a distance between the first identifier element 714a and the second identifier element 714b as a distance between the plunger and the barrel of the syringe.

As shown in FIG. 16A, at step 1606, process 1600 includes determining a temperature and/or a color of a fluid in the syringe. For example, image capture system 702 (e.g., sensor system 206, etc.), management system 102, and/or central computing system 202 may determine, based on the plurality of images, a temperature and/or a color of a fluid contained in the syringe. As an example, image capture system 702 may include a camera with a filter configured to capture the color in images and/or an IR camera configured to capture IR images.

As shown in FIG. 16A, at step 1608, process 1600 includes determining a type of the fluid. For example, image capture system 702 (e.g., sensor system 206, etc.), management system 102, and/or central computing system 202 may determine, based on the fluid flow rate of a fluid delivery and/or the temperature and/or the color of the fluid, a type of the fluid associated with at least one fluid delivery. In some non-limiting embodiments or aspects, image capture system 702 (e.g., sensor system 206, etc.), management system 102, and/or central computing system 202 may compare the temperature of the fluid contained in the syringe to a threshold temperature associated with the fluid (e.g., associated with the type of fluid, etc.) and, in response to determining that the temperature of the fluid contained in the syringe satisfies the threshold temperature, automatically control at least one medical device (e.g., an electronic valve, an infusion pump, etc.) to stop at least one fluid delivery from the syringe.

As shown in FIG. 16A, at step 1610, process 1600 includes determining a fluid delivery. For example, image capture system 702 (e.g., sensor system 206, etc.), management system 102, and/or central computing system 202 may determine, based on the location of the plunger of the syringe relative to the barrel of the syringe over the period of time, at least one fluid delivery from the syringe. In some non-limiting embodiments or aspects, determining the least one fluid delivery further includes determining, based on the location of the plunger of the syringe relative to the barrel of the syringe over the period of time, at least one of an amount of fluid delivered by the at least one fluid delivery and a fluid flow rate of the at least one fluid delivery.

As shown in FIG. 16A, at step 1612, process 1600 includes determining a location of a glove of a caregiver. For example, image capture system 702 (e.g., sensor system 206, etc.), management system 102, and/or central computing system 202 may determine, based on the plurality of images, a location of a glove of a caregiver within the environment over the period of time.

As show in FIG. 16B, at step 1614, process 1600 includes determining a flush technique associated with the fluid delivery. For example, image capture system 702 (e.g., sensor system 206, etc.), management system 102, and/or central computing system 202 may determine, based on the location of the glove of the caregiver within the environment over the period of time, a flush technique associated with the at least one fluid delivery, wherein the flush technique includes a pulsatile flush or a continuous flush.

As shown in FIG. 16B, at step 1616, process 1600 includes obtaining VAM data associated with the at least one determined event (e.g., event data, etc.). For example, image capture system 702 (e.g., sensor system 206, etc.), management system 102, and/or central computing system 202 may, based on the at least one determined event (e.g., a fluid delivery, etc.), update a database including events associated with the environment, provide an alert associated with the at least one determined event, and/or control one or more medical devices in the environment.

In some non-limiting embodiments or aspects, in response to the at fluid delivery, image capture system 702 (e.g., sensor system 206, etc.), management system 102, and/or central computing system 202 may at least one of: provide, to user device 208, an alert associated with the fluid delivery; and automatically control at least one medical device (e.g., a valve, an infusion pump, etc.) to stop a flow of fluid in a fluid flow path including the medical device associated with the at least one event.

Referring now to FIG. 17, FIG. 17 is a flowchart of non-limiting embodiments or aspects of a process 1700 for obtaining VAM data. In some non-limiting embodiments or aspects, one or more of the steps of process 1700 may be performed (e.g., completely, partially, etc.) by management system 102 (e.g., one or more devices of management system 102, etc.). In some non-limiting embodiments or aspects, one or more of the steps of process 1700 may be performed (e.g., completely, partially, etc.) by another device or a group of devices separate from or including management system 102, such as local system 104 (e.g., one or more devices of local system 104, etc.), central computing system 202 (e.g., one or more devices of central computing system 202, etc.), medication source system 204 (e.g., one or more devices of medication source system 204, etc.), sensor system 206 (e.g., one or more devices of sensor system 206, etc.), and/or user device 208 (e.g., one or more devices of a system of user device 208, etc.).

As shown in FIG. 17, at step 1702, process 1700 includes obtaining images. For example, image capture system 702 (e.g., sensor system 206, etc.), management system 102, and/or central computing system 202 may obtain images. As an example, image capture system 702 may capture, over a period of time, a plurality of images of an environment (e.g., environment 700, etc.) surrounding the one or more image capture devices. In such an example, management system 102 and/or central computing system 202 may obtain the plurality of images from image capture system 702 (e.g., sensor system 206, etc.).

As shown in FIG. 17, at step 1704, process 1700 includes determining a state of a package. For example, image capture system 702 (e.g., sensor system 206, etc.), management system 102, and/or central computing system 202 may determine, based on the plurality of images, a state of a package containing a medical device over the period of time. As an example, a state of a package may include an open package or a closed package (e.g., whether a medical device is removed from the package, etc.)

As shown in FIG. 17, at step 1706, process 1700 includes determining whether a medical device is removed from the package. For example, image capture system 702 (e.g., sensor system 206, etc.), management system 102, and/or central computing system 202 may determine, based on the state of the package over the period of time, whether the medical device is removed from the package. As an example, image capture system 702 (e.g., sensor system 206, etc.), management system 102, and/or central computing system 202 may determine, based on the state of the package, a type of medical device 712 included in the package and whether the medical device 712 has been removed from the package. In such an example, image capture system 702 (e.g., sensor system 206, etc.), management system 102, and/or central computing system 202 may determine, based on the type of the medical device and a time at which the medical device 712 is determined to be removed from the package, at least one event associated with the medical device, such as a first use of the medical device, a reuse of the medical device, a replacement of the medical device with a new medical device, and/or the like.

In some non-limiting embodiments or aspects, the package includes a removable first layer covering a second layer, wherein the first layer including a first color, wherein the second layer includes a second color different than the first color, and wherein removal of the first layer from the package reveals the second layer. In some non-limiting embodiments or aspects, a color of the package is configured to change when exposed to air. For example, image capture system 702 (e.g., sensor system 206, etc.), management system 102, and/or central computing system 202 may determine, based on a detected or determined color or layer of the package, whether the package has been opened and the medical device removed from the package. In such an example, the first layer may be at least partially transparent.

In some non-limiting embodiments or aspects, a portion of the package is transparent such that the medical device contained within the package is visible through the transparent portion of the package. For example, image capture system 702 (e.g., sensor system 206, etc.), management system 102, and/or central computing system 202 may determine, based on whether the medical device is detected or determined within the package, whether the package has been opened and the medical device removed from the package.

In some non-limiting embodiments or aspects, the package is associated with a first identifier element, the medical device is associated with a second identifier element different than the first identifier element, and the state of the package is determined based on a location of the first identifier element with respect to a location of the second identifier element. For example, image capture system 702 (e.g., sensor system 206, etc.), management system 102, and/or central computing system 202 may determine, based on a distance between the first identifier element and the second identifier element satisfying a threshold distance, whether the package has been opened and the medical device removed from the package.

In some non-limiting embodiments or aspects, image capture system 702 (e.g., sensor system 206, etc.), management system 102, and/or central computing system 202 may determine, based on the plurality of images, a location of a glove of a caregiver within the environment over the period of time with respect to a location of the package, and determine the state of the package based on the location of the glove of the caregiver within the environment over the period of time with respect to the location of the package.

In some non-limiting embodiments or aspects, the package includes a removable first layer covering a second layer, the removable first layer includes a first identifier element, the second layer includes a second identifier element, and the removable first layer is at least partially transparent. For example, management system 102 may determine the state of the package by: determining, based on the plurality of images, a distance between the first identifier element and the second identifier element; determining, based on the distance between the first identifier element and the second identifier element, whether the package is faulty; and in response to determining that the package is faulty, provide, to a user device, an alert associated with the faulty package.

In some non-limiting embodiments or aspects, management system 102 may determine, based on the plurality of images, a plurality of locations of a plurality of medical devices 712 within the environment over the period of time and a plurality of types of the plurality of medical devices 712; and determine, based on the plurality of locations of the plurality of medical devices 712 within the environment over the period of time, the plurality of types of the plurality of medical devices 712, and the state of the package over the period of time, at least one event of the following events: (i) a reuse of the medical device including a connection of the medical device to two or more medical devices in the environment over the period of time and (ii) a replacement of the medical device with a new medical device of a same type as the medical device in the environment.

As shown in FIG. 17, at step 1708, process 1700 includes obtaining VAM data associated with the determination that the medical device is removed from the package (e.g., event data, medical device data, etc.). For example, image capture system 702 (e.g., sensor system 206, etc.), management system 102, and/or central computing system 202 may update, based on a determination that the medical device is removed from the package, a database including medical devices in the environment. As an example, image capture system 702 (e.g., sensor system 206, etc.), management system 102, and/or central computing system 202 may determine, based on a determination that the medical device is removed from the package and/or a time associated therewith, whether an event associated with the medical device includes a first use of the medical device or a reuse of the medical device. In such an example, image capture system 702 (e.g., sensor system 206, etc.), management system 102, and/or central computing system 202 may automatically document the first use and any reuse of a medical device, alert a nurse (e.g., via user device 208, etc.) that the medical device is being improperly reused, and/or control a medical device (e.g., a valve, an infusion pump, etc.) to stop of flow of fluid through a fluid flow path associated with the reused medical device, which may improve patient safety and/or reduce costs associated with complications arising from reuse of medical devices.

Referring now to FIG. 18, FIG. 18 is a flowchart of non-limiting embodiments or aspects of a process 1800 for obtaining VAM data. In some non-limiting embodiments or aspects, one or more of the steps of process 1800 may be performed (e.g., completely, partially, etc.) by management system 102 (e.g., one or more devices of management system 102, etc.). In some non-limiting embodiments or aspects, one or more of the steps of process 1800 may be performed (e.g., completely, partially, etc.) by another device or a group of devices separate from or including management system 102, such as local system 104 (e.g., one or more devices of local system 104, etc.), central computing system 202 (e.g., one or more devices of central computing system 202, etc.), medication source system 204 (e.g., one or more devices of medication source system 204, etc.), sensor system 206 (e.g., one or more devices of sensor system 206, etc.), and/or user device 208 (e.g., one or more devices of a system of user device 208, etc.).

As shown in FIG. 18, at step 1802, process 1800 includes obtaining images. For example, image capture system 702 (e.g., sensor system 206, etc.), management system 102, and/or central computing system 202 may obtain images. As an example, image capture system 702 may capture, over a period of time, a plurality of images of an environment (e.g., environment 700, etc.) surrounding the one or more image capture devices. In such an example, management system 102 and/or central computing system 202 may obtain the plurality of images from image capture system 702 (e.g., sensor system 206, etc.).

As shown in FIG. 18, at step 1804, process 1800 includes obtaining auxiliary data. For example, image capture system 702 (e.g., sensor system 206, etc.), management system 102, and/or central computing system 202 may obtain auxiliary data. As an example, image capture system 702 (e.g., sensor system 206, etc.), management system 102, and/or central computing system 202 may obtain, from a data source other than the one or more image capture devices (e.g., from an auxiliary system, etc.), auxiliary data associated with at least one of: at least one image of the environment during the period of time and audio recorded in the environment during the period of time.

In some non-limiting embodiments or aspects, the medical device includes a catheter, and the auxiliary data includes at least one of: a tubing type of the catheter, a size of the catheter, a shape of the catheter, and a location of a catheter insertion site of the catheter on a patient.

In some non-limiting embodiments or aspects, the auxiliary data is associated with the audio recorded in the environment during the period of time. For example, the audio may include a predetermined signal associated with the medical device. As an example, the medical device may include an infusion pump, and the predetermined signal may include an audible signal emitted by the infusion pump (e.g., a power on sound, an indicator sound, etc.).

As shown in FIG. 18, at step 1806, process 1800 includes determining a location, a type, a trajectory, and/or an orientation of a medical device. For example, image capture system 702 (e.g., sensor system 206, etc.), management system 102, and/or central computing system 202 may determine, based on the plurality of images and the auxiliary data, a location, a type, a trajectory, and/or an orientation of a medical device within the environment over the period of time. In some non-limiting embodiments or aspects, the medical device may not be visible (e.g., may not be detectable, etc.) in at least a portion of the plurality of images.

In some non-limiting embodiments or aspects, image capture system 702 (e.g., sensor system 206, etc.), management system 102, and/or central computing system 202 may determine a plurality of locations of a plurality of medical devices 712 within the environment over the period of time and a plurality of types of the plurality of medical devices 712. For example, the plurality of medical devices 712 may include the medical device and the at least one other medical device, and the medical device and the at least one other medical device may be configured to emit a predetermined audible signal when connected (and/or disconnected). As an example, image capture system 702 (e.g., sensor system 206, etc.), management system 102, and/or central computing system 202 may determine, based on the plurality of locations of the plurality of medical devices 712 within the environment over the period of time and the plurality of types of the plurality of medical devices 712, at least one connection (and/or disconnection) between two or more medical devices of the plurality of medical devices 712 and/or update, based on the at least one connection determined between the two or more medical devices, a database. In such an example, the audio included in the auxiliary data may include the predetermined audible signal emitted when the medical device and the at least one other medical are connected (and/or disconnected).

As shown in FIG. 18, at step 1808, process 1800 includes obtaining VAM data associated with a location, a type, a trajectory, and/or an orientation of a medical device (e.g., event data, medical device data, etc.). For example, image capture system 702 (e.g., sensor system 206, etc.), management system 102, and/or central computing system 202 may, based on the at least one determined event, update a database including events associated with the environment, provide an alert associated with the at least one determined event, and/or control one or more medical devices in the environment.

In some non-limiting embodiments or aspects, in response to the at fluid delivery, image capture system 702 (e.g., sensor system 206, etc.), management system 102, and/or central computing system 202 may at least one of: provide, to user device 208, an alert associated with the fluid delivery; and automatically control at least one medical device (e.g., a valve, an infusion pump, etc.) to stop a flow of fluid in a fluid flow path including the medical device associated with the at least one event.

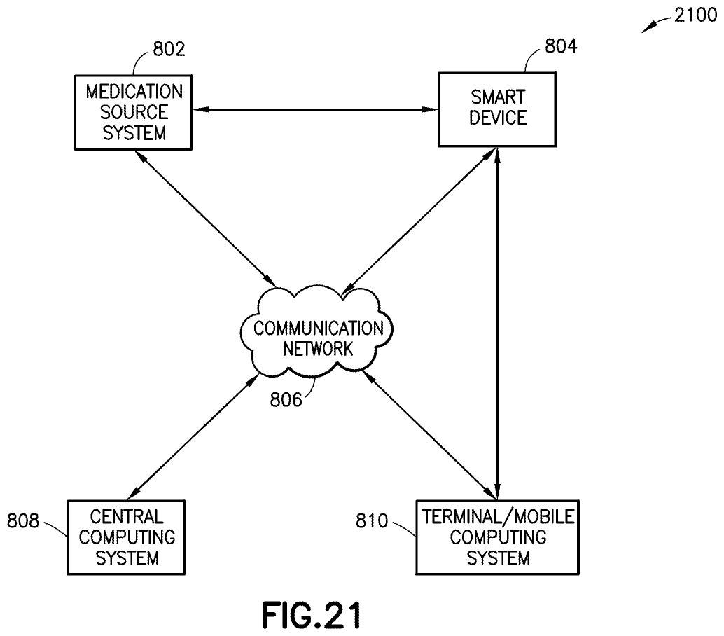

Referring now to FIG. 21, FIG. 21 is a diagram of non-limiting embodiments or aspects of an implementation of an environment 2100 of a local system 104 in which systems, devices, products, apparatus, and/or methods, described herein, can be implemented. For example, as shown in FIG. 21, environment 2100 includes medication source system 802, smart device 804, communication network 806, central computing system 808, and terminal/mobile computing system 810. Systems and/or devices of environment 2100 can interconnect via wired connections, wireless connections, or a combination of wired and wireless connections. In the implementation of the environment 2100 shown in FIG. 21, medication source system 802 may be the same as or similar to medication source system 204, communication network 806 may be the same as or similar to communication network 106, central computing system 808 may be the same as or similar to management system 102 and/or central computing system 202, and/or terminal/mobile computing system 810 may be the same as or similar to user device 208.

In some non-limiting embodiments or aspects, medication source system 802 includes one or more devices capable of delivering one or more fluids to one or more lumens (e.g., fluid lines, IV lines, etc.). For example, medication source system 802 may include one or more manual fluid delivery systems (e.g., one or more IV bags, one or more syringes, etc.) and/or an infusion pump system including one or more infusion pumps. In some non-limiting embodiments, smart device 804 may include a plurality of smart devices 804 (e.g., one or more other and/or differ types of smart devices 804, etc.).

In some non-limiting embodiments or aspects, smart device 804 includes one or more devices capable of receiving information and/or data from medication source system 802, one or more other smart devices 804, communication network 806, central computing system 808, and/or terminal/mobile computing system 810 and/or communicating information and/or data to medication source system 802, one or more other smart devices 804, communication network 806, central computing system 808, and/or terminal/mobile computing system 810. For example, smart device 804 may include one or more computing systems including one or more processors (e.g., one or more computing devices, one or more mobile computing devices, etc.). In some non-limiting embodiments or aspects, smart device 804 may be capable of receiving information (e.g., from medication source system 802 (e.g., from medication source controller 904 and/or from medication source device 906, etc.), from terminal/mobile computing system 810, from one or more other smart devices 804, etc.) via a short range wireless communication connection (e.g., an NFC or proprietary communication connection, an RFID communication connection, a Bluetooth® communication connection, and/or the like), and/or communicating information (e.g., to medication source system 802 (e.g., to medication source controller 904 and/or to medication source device 906, etc.), to terminal/mobile computing system 810, to one or more other smart devices 804, etc.) via a short range wireless communication connection.

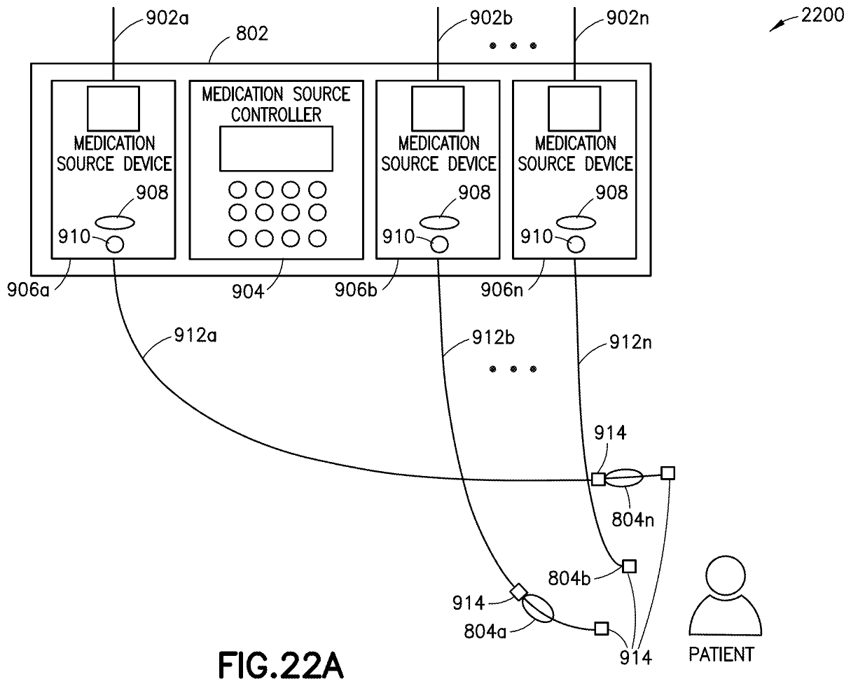

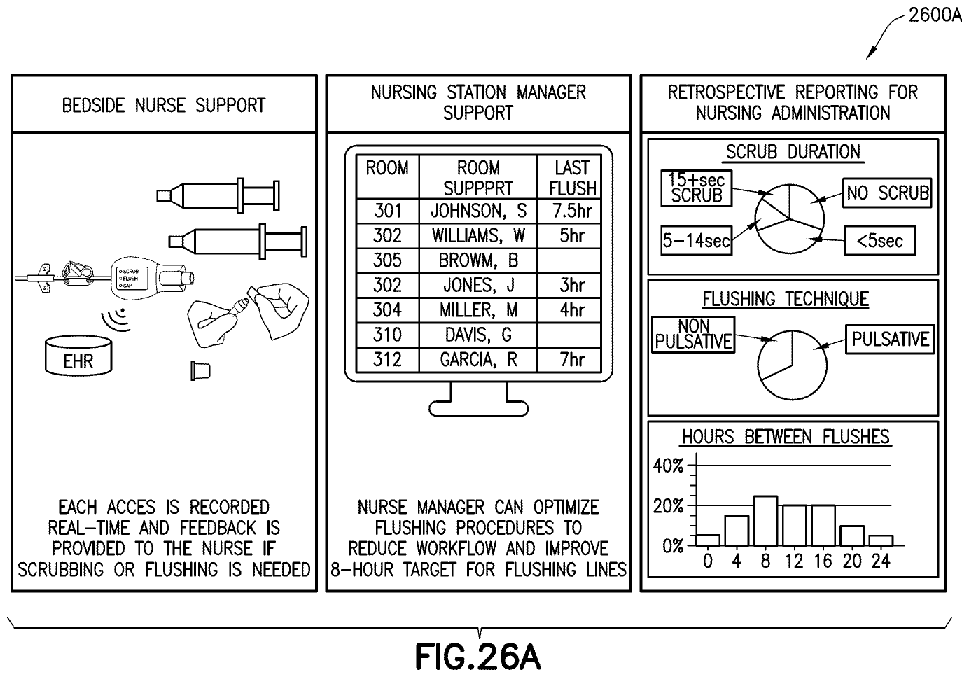

In some non-limiting embodiments or aspects, as shown in FIG. 26B, smart device 804 may provide direct patient-side feedback (e.g., via an LED light to a nurse, etc.) in response to (i) detecting that needleless connector 914 and/or lumen 912 thereof has not been scrubbed for a predetermined period of time and/or before a scheduled use, (ii) detecting that needleless connector 914 and/or lumen 912 thereof has not been scrubbed for a sufficient period of time prior to accessing a catheter line, (iii) detecting that a flush of needleless connector 914 and/or lumen 912 is due, (iv) detecting that a disinfection cap was not attached after a previous access to needleless connector 914 and/or lumen 912, and/or the like. For example, smart device 804 may include needleless connector 914, and needleless connector 914 may be configured to detect at least one of a scrubbing event, a flushing event, a connection or capping event, or any combination thereof. As an example, and needleless connector 914 may be configured to provide information and/or data associated with a detected scrubbing event, a detected flushing event, a detected connection or capping event, and/or a detected disconnection event (e.g., with processor 204, memory 206, storage component 208, input component 210, output component 212, etc.) to store events and report compliance performance for compliance event monitoring. Further details regarding non-limiting embodiments or aspects of smart device 804 are provided below FIGS. 22A-22C, 23, 24A-24C, 25A-25C, 26A, 26B, and 27.

In some non-limiting embodiments or aspects, terminal/mobile computing system 810 includes a nurse station in a hospital. For example, as shown in an implementation 2600A in FIG. 26A, terminal/mobile computing system 810 may provide bedside nurse support (e.g., recordation of each access to needleless connector 914 and/or lumen 912 in real-time and feedback to a nurse if scrubbing or flushing is determined to be due or needed according to the recorded access, etc.), nursing station manager support (e.g., optimization of flushing procedures to reduce workflow and improve timed targets for flushing a needleless connector 914 and/or lumen 912, etc.), retrospective reporting for nursing administration (e.g., a scrub duration, a flushing technique, a time between flushes, and/or the like for a needleless connector 914 and/or lumen 912, etc.), and/or the like.

Referring now to FIGS. 22A-22C, FIGS. 22A-22C are diagrams of non-limiting embodiments or aspects of an implementation 2200 of one or more systems and/or one or more devices of FIG. 21. As shown in FIGS. 22A and 22C, medication source system 802 may include a medication source controller 904 and/or one or more medication source devices 906 (e.g., a plurality of mediation source devices 906a, 906b, . . . 906n, etc.). As an example, medication source controller 904 may include an infusion pump controller and/or medication source device 906 may include an infusion pump. In such an example, medication source system 802 may include the BD Alaris™ system. For example, medication source system 802 may include a BD Alaris™ PC Unit and one or more BD Alaris™ Pump Modules. As another example, medication source controller 904 may include a bed-side console or computing device, which may be separate from an infusion pump system, and/or medication source device 906, which may be separate from an infusion pump, may be associated with and/or connected to a medication source (e.g., an IV bag, a syringe, an end of an IV line connected and proximal to an IV bag or a syringe, etc.).

As shown in FIG. 22A, the plurality of medication source devices 906a, 906b, . . . 906n may be connected to a plurality of lumens (e.g., fluid lines, etc.) 902a, 902b, . . . 902n (e.g., for receiving a fluid and/or a medication at medication source system 802) and/or a plurality of lumens (e.g., fluid lines, etc.) 912a, 912b, . . . 912n (e.g., for delivering a fluid and/or a medication from medication source system 802, etc.). As shown in FIG. 22C, medication source device 906 may include pairing input 908 (e.g., a button, input component 210, etc.) and/or visual indicator 910 (e.g., a multi-color LED(s), output component 212, etc.). As shown in FIGS. 22A and 22B, the plurality of lumens 912a, 912b, . . . 912n may be connected to a plurality of smart devices 804a, 804b, . . . 804n.

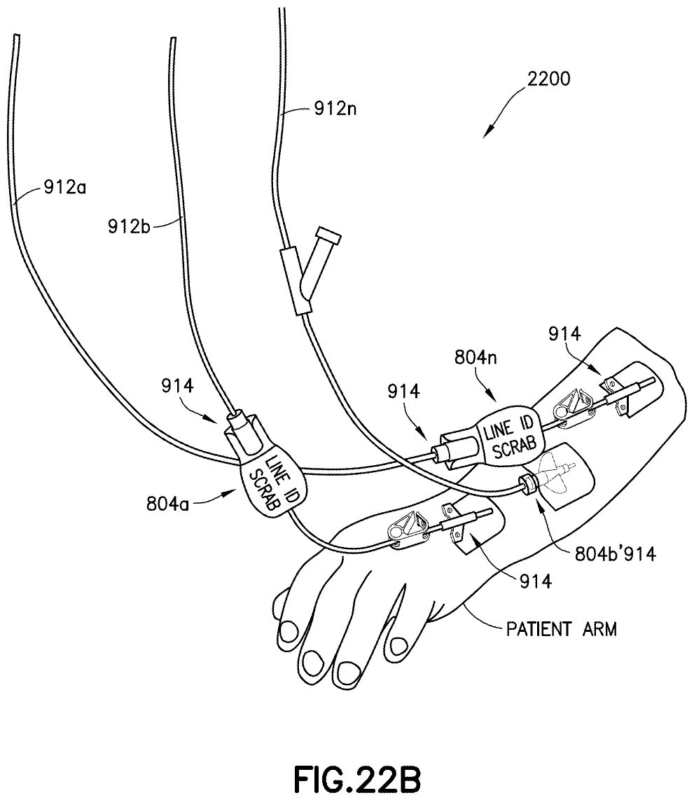

In some non-limiting embodiments or aspects, smart device 804 is configured to be removably connected to needleless connector 914 and/or a portion of lumen 912 proximate needleless connector 914, such as an IV lumen (e.g., a peripherally inserted central catheter (PICC), a peripheral intravenous catheter (PIVC), a central venous catheter (CVC), etc.), and/or the like. For example, smart device 804 may include a clamp, an adhesive, a frictional fit, and/or other attachment means configured to removably connect smart device 804 to needleless connector 914 and/or lumen 912 proximate needleless connector 914. As an example, as shown in FIGS. 22A and 22B, smart device 804a may be connected to needleless connector 914 and/or a catheter lumen that connects a catheter to lumen 912b, and/or smart device 804n may be connected to needleless connector 914 and/or a catheter lumen that connects a catheter to lumen 912a. In some non-limiting embodiments or aspects, smart device 804 includes needleless connector 914. For example, smart device 804 may be integrated with needleless connector 914 (e.g., within needleless connector 914 and/or within a catheter hub of a needleless connector of a fluid invasive device, etc.). As an example, as shown in FIGS. 22A and 22B, smart device 804b may include needleless connector 914 and/or a catheter hub that connects a catheter lumen to lumen 912n via a Y-site connector. In such an example, smart device 804 may include needleless connector 914 including housing 1102 of needleless connector 914 within housing 950 (e.g., integrated with housing 950, encompassed within housing 950, etc.). For example, needleless connector 914 may embed housing 950, smart device 804, and/or components thereof within housing 1102 of needleless connector 914 (or vice-versa) or housing 950, smart device 804, and/or components thereof connected to housing 1102. An advantage of adding sensors to standard designs is that the clinically validated performance characteristics and regulatory filings do not change. Sterilization techniques that are optimum for fluid-path components, may not be ideal for electronic devices and, therefore, therefore designs that do not change validated components that can be added later in manufacturing and assembly, or snapped on by the end-user, may have advantages. FIG. 23 is an implementation 2300 of non-limiting embodiments or aspects of a smart device 804.

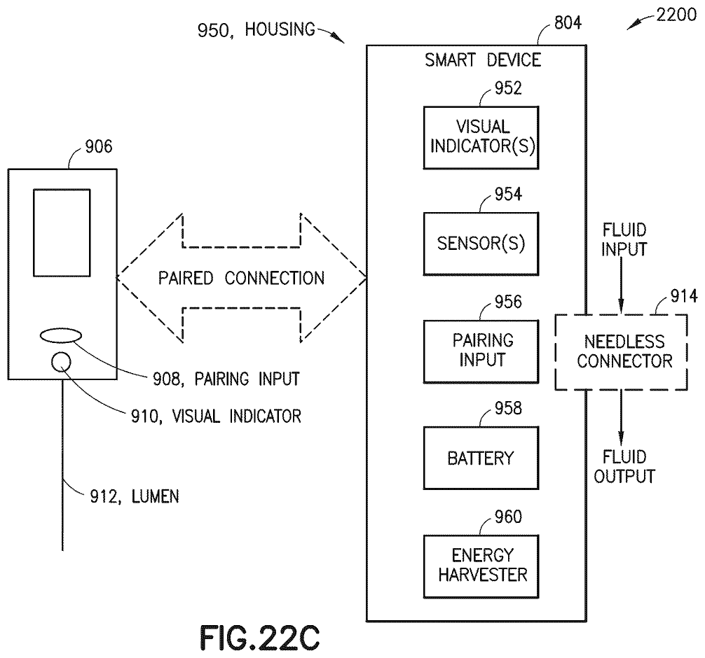

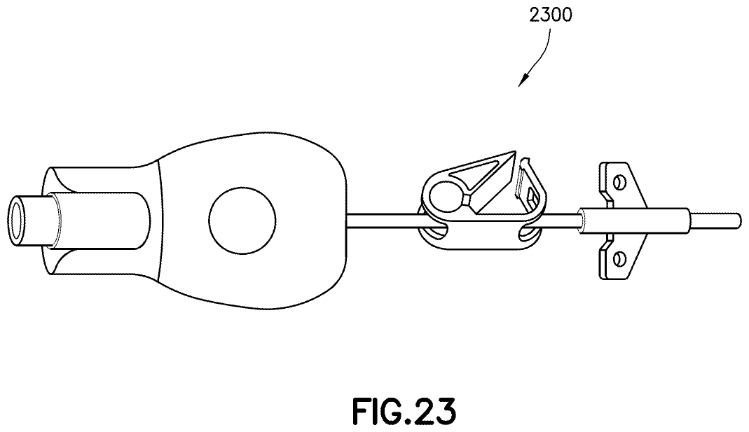

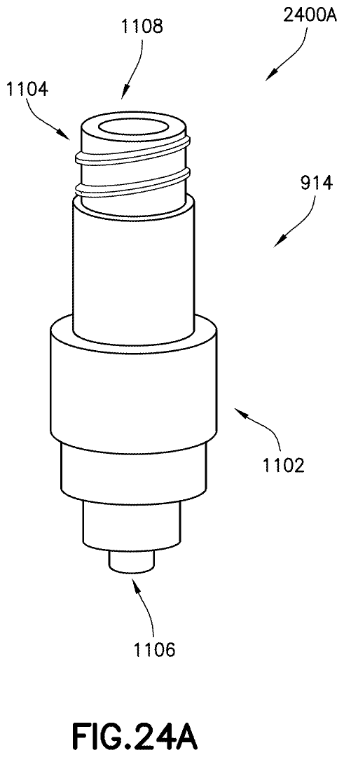

Referring also to FIG. 24A, FIG. 24A is a side view of non-limiting embodiments or aspects of an implementation 2400A of a needleless connector 914. As shown in FIG. 24A, a needleless connector 914 may include a fluid flow path in a housing 1102 between an inlet 1104 and an outlet 1106 opposite the inlet 1104. Inlet 1104 may be fluidically sealed by a displaceable septum 1108 configured to be displaced to open or connect inlet 1104 to the fluid flow path in response to connection of needleless connector 914 to a medical device (e.g., an infusion pump, an IV bag, a syringe, an IV line, etc.). For example, the needleless connector 914 may include the BD MaxPlus™ connector, the BD MaxZero™ needle-free connector, and/or the like. However, non-limiting embodiments or aspects are not limited thereto, and the needleless connector 914 may include any needleless connector 914 for use in fluid administration. For example, needleless connector 914 may include a port, a manifold, a stopcock, an open connector, a luer connector, and/or any other connector that does not rely on (but may or may not include) a needle to form a connection with a device and/or a patient. In some non-limiting embodiments or aspects, one or more components of smart device 804 may be included within housing 950 of needleless connector 914. For example, housing 1102 of needleless connector 914 may include housing 950 of smart device 804 (e.g., housing 950 may be integrated with housing 1102, encompassed within housing 1102, etc.).

As shown in FIG. 22C, smart device 804 may include visual indicator 952 (e.g., one or more visual indictors, a plurality of visual indicators, a multi-color LED(s), a plurality of LEDs, output component 212, etc.), sensor 954 (e.g., one or more sensors, a plurality of sensors, a sensor suite, etc.), pairing input 956 (e.g., one or more buttons, one or more force sensors, one or more accelerometers, input component 210, etc.), battery 958, and/or energy harvester 960 (e.g., a thermoelectric energy harvester, a photovoltaic energy harvester, a piezoelectric energy harvester, etc.). Visual indicator 952, sensor 954, pairing input 956, battery 958, energy harvester 960 and all or a portion of needleless connector 914 may be included within housing 950 of smart device 804. Visual indicator 952 may be visible through and/or extend from a sidewall of housing 950. Battery 958 and/or energy harvester 960 may provide power for operating components of smart device 804, such as visual indicator 952, sensor 954, pairing input 956, a rechargeable battery of battery 958, one or more components of device 200 included in smart device 804, and/or the like.

In some non-limiting embodiments or aspects, smart device 804 may include a label (e.g., a human readable label, etc.) that characterizes visual indicator 952 of smart device 804. For example, as shown in implementation 2400C in FIG. 24C, smart device 804 may include labels associated with visual indicators 952 (e.g., on a sidewall of housing 950, etc.) that characterize each visual indicator 952 as configured for providing an indication associated a particular event, such as one of: a scrubbing event in which needleless connector 914 is scrubbed with a disinfectant (e.g., a label “SCRUB”, etc.); a flushing event in which needleless connector 914 is flushed with a solution (e.g., a label “FLUSH”, etc.); a connection or capping event in which needleless connector 914 is connected to a medical device (e.g., a label “CAP”, etc.); and/or the like. In some non-limiting embodiments or aspects, smart device 804 may include a single visual indicator 952 (e.g., as shown in implementation 2400B in FIG. 24B). For example, smart device 804 may control single visual indicator 952 to illuminate in a particular color and/or in a particular pattern to provide an indication or prompt to a user, such as to illuminate a continuous green in response to sensing that scrubbing of needleless connector 914 has occurred for a predetermined period of time (e.g., 15 seconds, etc.), to illuminate a pulsating green in response to sensing that a proper pulsatile flush has occurred, to illuminate a pulsating red in response to determining that a pulsatile flush of needleless connector 914 has not occurred for a predetermined period of time (e.g., 8 hours, etc.), to illuminate a continuous red in response to determining that needleless connector 914 has not been capped with a disinfectant cap for a predetermined period of time (e.g., over minutes, etc.).

In some non-limiting embodiments or aspects, communication circuitry (e.g., communication interface 214, etc.) of medication source device 906 is configured to establish communication with communication circuitry (e.g., communication interface 214, etc.) of smart device 804 based on user input to pairing input 908 of medication source device 906 and user input to pairing input 956 of smart device 804. For example, medication source device 906 may establish a short range wireless communication connection (e.g., an NFC communication connection, an RFID communication connection, a Bluetooth® communication connection, etc.) with smart device 804. As an example, visual indicator 910 may be configured to emit a predetermined light pattern (e.g., to blink rapidly to indicate that medication source device 906 is in a pairing mode, etc.) in response to a predetermined user input to pairing input 908 (e.g., in response to a user pressing and holding a button of pairing input 908, etc.) of medication source device 906. In such an example, smart device 804 may be configured to establish communication with medication source device 906 (e.g., pair and/or activate a pairing sequence for pairing smart device 804 with medication source device 906, etc.) in response to a predetermined user input to paring input 956 (e.g., in response to a user pressing and holding a button of pairing input 956, etc.) of smart device 804 at a same time that medication source device 906 is in the pairing mode.

In some non-limiting embodiments or aspects, when medication source device 906 is paired with smart device 804, visual indicator 910 of medication source device 906 and visual indicator 952 of smart device 804 are configured to provide a same type of visual output (e.g., a same color of light from a multi-colored LED, a same pattern of light, etc.). For example, and referring again to FIG. 22A, medication source device 906a may be paired with smart device 804n and each of medication source device 906a and smart device 804n may output a first color of light (e.g., red light), medication source device 906b may be paired with smart device 804a and each of medication source device 906b and smart device 804a may output a second color of light (e.g., green light), medication source device 906n may be paired with smart device 804b and each of medication source device 906n and smart device 804b may output a third color of light (e.g., blue light), and/or the like.

In some non-limiting embodiments or aspects, sensor 954 includes at least one of: one or more force sensors (e.g., one or more piezoelectric elements or transducers, one or more force sensitive resistive (FSR) sensors, one or more strain gauges, etc.); one or more accelerometers; one or more gyroscopes; one or more pressure sensors; one or more acoustic sensors (e.g., an acoustic sensor configured to detect a sound signature associated with a type, a state, and/or an operation of a medical device, etc.); one or more optical sensors (e.g., an optical sensor configured to detect at least one of a movement of a septum, a color signature and a reflectance of a medical device connected to smart device 804, etc.), one or more identification sensors (e.g., an identification sensor configured to detect an identification tag on a medical device connected to or being connected to the needleless connector 914, such as a magnetometer configured to detect a magnetic material, a barcode scanner configured to read a bar code, etc.); one or more position sensors (e.g., a position sensor configured to detect movement of smart device 804, etc.); one or more RBG color sensors; one or more mechanical switches; one or more flow sensors (e.g., an ultrasonic flow sensor, a thermal flow sensor, etc.); or any combination thereof.

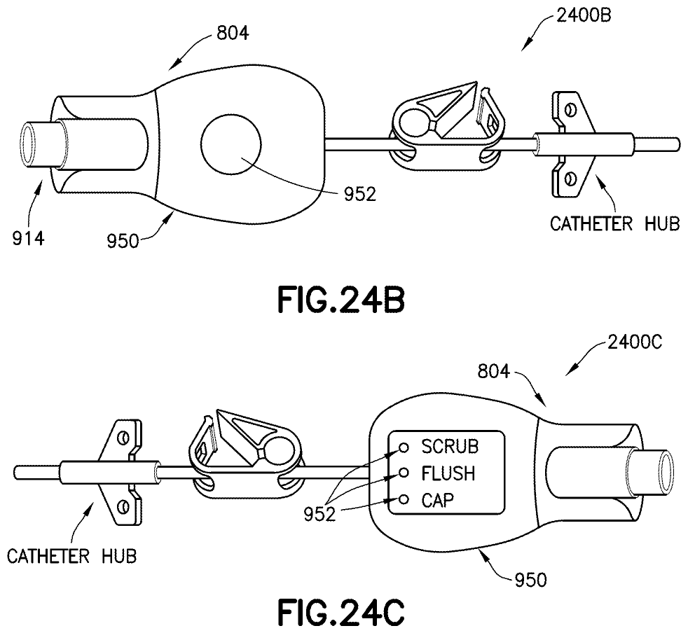

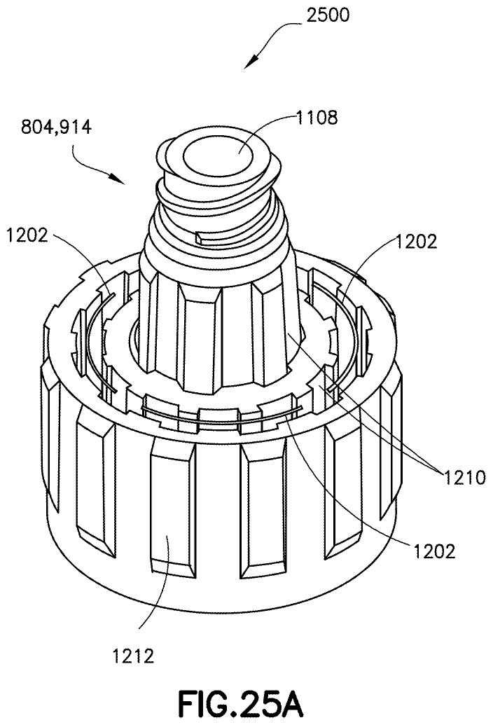

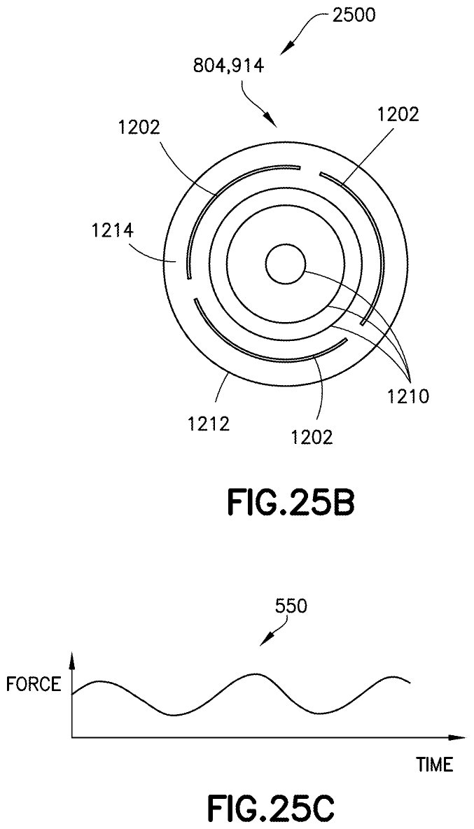

FIG. 25A is a perspective view and FIG. 25A is a top view of non-limiting embodiments or aspects of an implementation 2500 of smart device 804 including needleless connector 914. Referring also to FIG. 24A, needleless connector 914 may include a fluid flow path in housing 1102 between inlet 1104 and outlet 1106 opposite the inlet 1104. Inlet 1104 may be fluidically sealed by displaceable septum 1108 configured to be displaced to open or connect inlet 1104 to the fluid flow path in response to connection of needleless connector 914 to a medical device (e.g., an infusion pump, an IV bag, a syringe, an IV line, etc.). Referring again to FIGS. 25A and 25B, in some non-limiting embodiments, smart device 804 may include sensor 954. For example, sensor 954 may include force sensor 1202 connected to needleless connector 914. As an example, force sensor 1202 may be configured to sense, detect, and/or determine a force signal. In such an example, at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof, may be determined based on the force signal (e.g., by smart device 804, etc.). In such an example, a pattern of events including a plurality of the least one of: the scrubbing event in which the needleless connector is scrubbed with the disinfectant, the flushing event in which the needleless connector is flushed with the solution, the connection event in which the needleless connector is connected to the medical device, the disconnection event in which the needleless connector is disconnected from the medical device, a time between one or more detected events (e.g., a dwell or connection time during which the needleless connector is connected to medical device between a connection event and a disconnection event, etc.), or any combination thereof may be determined based on the force signal, and a medication administration event in which a medication is administered to a patient via needleless connector 914 may be determined based on the pattern of events. As an example, a standard medical practice may assume a Scrub-Flush-Scrub-MedAdmin-Scrub-Flush-Scrub pattern or sequence of events and, therefore, detection of three access of luer connectors may be interpreted by smart device 804 as a medication administration event. For example, FIG. 25C is a graph 550 of non-limiting embodiments or aspects of a force measurement or signal over time. As shown in FIG. FIG. 25C, pulsatile flushing may be determined or detected by force measurement, for example, when flushing is achieved by intermittent pressure pulses applied to a plunger of a flush syringe, and smart device 804 can detect occurrences of pulsatile flushes by identifying periodic force signals between x-y Hz in a force signal perpendicular to a surface of septum 1108 of needleless connector 914. For example, smart device 804 may determine, based on the force signal indicating periodic forces in the second direction perpendicular to the surface of the septum facing in the first direction, the flushing event, and that the flushing event includes a pulsatile flushing event.

In some non-limiting embodiment or aspects, smart device may 804 may include communication circuitry configured to transmit the force signal to a remote computing system. For example, medication source system 802, central computing system 808, and/or terminal/mobile computing system 810 may obtain the force signal from smart device 804 and/or needleless connector 914 and process the force signal to determine at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof.

In some non-limiting embodiments or aspects, force sensor 1202 includes at least one of: a piezoelectric element, a force sensitive resistive (FSR) sensor, a strain gauge, or any combination thereof. In some non-limiting embodiments or aspects, force sensor 1202 is positioned between an outer surface of inner wall 1210 (e.g., an inner harder plastic wall) of needleless connector 914 defining the fluid flow path of needleless connector 914 and an inner surface of an outer wall 1212 (e.g., a softer, a more flexible, a more pliable, a rubber, etc. wall) of needleless connector 914 surrounding the inner wall 1210 of needleless connector 914. In some non-limiting embodiments or aspects, an area between an outer surface of inner wall 1210 (e.g., an inner harder plastic wall) of needleless connector 914 defining the fluid flow path of needleless connector 914 and an inner surface of an outer wall 1212 (e.g., a softer, a more flexible, more, a more pliable, a rubber, etc. wall) of needleless connector 914 surrounding the inner wall 1210 of needleless connector 914, which may be held by a user during cleaning and/or connection to another medical device, may be filled with a rubber or other pliable type material 1214 including force sensors 1202 as force sensing films within the material 1214 between the inner wall 1210 and the outer wall 1212. In some non-limiting embodiments or aspects, force sensors 1202 may be located between inner wall 1210 and outer wall 1212 below threading on and/or proximal to inlet 1104 of needleless connector 914.

In some non-limiting embodiments or aspects, force sensor 1202 includes a plurality of force sensors 1202 positioned around the fluid flow path of needleless connector 914 between the outer surface of inner wall 1210 of needleless connector 914 defining the fluid flow path of needleless connector 914 and the inner surface of outer wall 1212 of needleless connector 914 surrounding inner wall 1210 of needleless connector 914. For example, inlet 1104 of needleless connector 914 may include septum 1108 including a surface facing in a first direction, and force sensor 1202 may be configured to detect a force in a second direction perpendicular to the surface of the septum facing in the first direction. As an example, the flushing event, which may include a pulsatile flushing event, may be determined based on the force signal indicating periodic forces in the second direction perpendicular to the surface of the septum facing in the first direction.

In some non-limiting embodiments or aspects, sensor 954 includes a pressure sensor, and the pressure sensor is one of: in direct contact with a fluid in the fluid flow path of the needleless connector; located within an inner wall of the needleless connector defining the fluid flow path of the needleless connector, and located within a wall of a lumen connected to the needleless connector. For example, smart device 804 may determine or detect pulsatile flush, a flush, and or a med-administration by the pressure sensor in contact with the fluid path in the needleless connector 914 and/or a lumen thereof.

In some non-limiting embodiments or aspects, sensor 954 includes an optical sensor configured to detect at least one of a color signature and a reflectance of a medical device connected to and/or being connected to needleless connector 914, and smart device 804 may determine a type of the medical device based on the at least one of the color signature and the reflectance of the medical device. For example, a color signature and/or the reflectance of the medical device may be indicative of a syringe, an IV bag, an infusion pump, and/or a particular type thereof.

In some non-limiting embodiments or aspects, sensor 954 includes an identification sensor configured to detect an identification tag on a medical device connected to or being connected to the needleless connector. For example, the identification sensor may include a magnetometer, and the identification tag may include a magnetic material on and/or integrated with needleless connector 914.

In some non-limiting embodiments or aspects, sensor 954 includes a position sensor configured to detect movement of the needleless connector. For example, a movement of the patient, a fall event of the patient, a movement of a bed of the patient may be determined (e.g., by smart device 804, etc.) based on the detected movement of the needleless connector.

In some non-limiting embodiments or aspects, sensor 954 includes an RGB color sensor configured to detect a color of a fluid in the fluid flow path of the needleless connector. For example, at least one of a blood-draw in the needleless connector and a retention of blood in the needleless connector may be determined (e.g., by smart device 804, etc.) based on the color of the fluid detected in the fluid flow path of the needleless connector.

In some non-limiting embodiments or aspects, smart device 804 including needleless connector 914 may include visual indicator 952, and visual indicator 952 may be configured to provide a visual indication associated with the at least one of: the scrubbing event in which the needleless connector is scrubbed with the disinfectant, the flushing event in which the needleless connector is flushed with the solution, the connection event in which the needleless connector is connected to the medical device, the disconnection event in which the needleless connector is disconnected from the medical device, or any combination thereof. For example, as shown in an implementation 2600B in FIG. 26B, smart device 804 may provide direct patient-side feedback (e.g., via an LED light to a nurse, etc.) in response to (i) detecting that needleless connector 914 and/or lumen 912 thereof has not been scrubbed for a predetermined period of time and/or before a scheduled use, (ii) detecting that needleless connector 914 and/or lumen 912 thereof has not been scrubbed for a sufficient period of time prior to accessing a catheter line, (iii) detecting that a flush of needleless connector 914 and/or lumen 912 is due, (iv) detecting that a disinfection cap was not attached after a previous access to needleless connector 914 and/or lumen 912, and/or the like. For example, smart device 804 may include needleless connector 914, and needleless connector 914 may be configured to detect at least one of a scrubbing event, a flushing event, a connection or capping event, or any combination thereof. As an example, and needleless connector 914 may be configured to provide information and/or data associated with a detected scrubbing event, a detected flushing event, and/or a detected connection or capping event (e.g., with processor 204, memory 206, storage component 208, input component 210, output component 212, etc.) to store events and report compliance performance for compliance event monitoring.

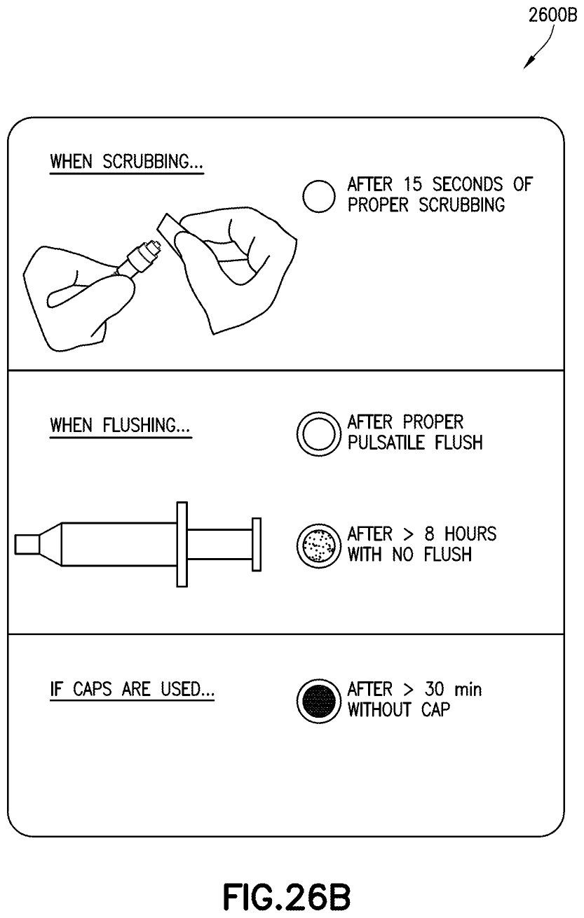

FIG. 27 is a diagram of non-limiting embodiments or aspects of an implementation 2700 of a smart device for detecting an extravasation or an infiltration of a medication in a catheter. As shown in FIG. 27, smart device 804 may be connected to or integrated with a needleless connector 914 at a catheter hub of catheter 1402 including a catheter lumen or line 1404 and a needle tip 1406 for delivering fluid to a patient at an opposite end of the catheter line 1404 from smart device 804. Catheter 1402 may be inserted in a blood vessel (e.g., a vein, an artery, etc.) of the patient. For example, the location of the tip 1406 of the needle may be within the blood vessel of the patient, within a wall of the blood vessel or a wall of the urinary tract of the patient, or outside the blood vessel or the urinary tract and the wall of the blood vessel or the wall of the urinary tract of the patient. In some non-limiting embodiments or aspects, smart device 804 including catheter 1402 may include a wired and/or a wireless transmitted configured to (e.g., via a wire, wirelessly, etc.) transmit the at least one signal (and/or a variation in the at least one signal over a period of time, a location of the tip of the needle with respect to a blood vessel or a urinary tract of the patient, etc.) to a remote computer system or processing device. However, in some non-limiting embodiments or aspects, catheter 1402 may be configured for insertion in a blood vessel.

In some non-limiting embodiments or aspects, smart device 804 may include sensor 954 located outside a body of the patient (e.g., at needleless connector 914 at the hub of catheter 1402 located outside of a body of the patient, and sensor 954 may be connected to the hub of catheter 1402 outside the body of the patient, etc.). For example, sensor 954 may include at least one of a pressure sensor and an acoustic sensor (e.g., a piezoelectric transducer, etc.). As an example, sensor 954 including the pressure sensor and/or the acoustic sensor may be connected to catheter 1402 at needleless connector 914 at the hub of catheter 1402. For example, the hub of catheter 1402 may include needleless connector 914 and/or smart device 804, and sensor 954 may be included in needleless connector 914. In such an example, sensor 954 may be configured to sense, detect, and/or measure a pressure signal, an acoustic signal, and/or temporal variations in the pressure signal and/or the acoustic signal with the catheter needle in the body of the patient. For example, the pressure signal and/or the acoustic signal sensed by sensor 954 may be transmitted through a fluid in the catheter and/or through material of the catheter (e.g., via needle tip 1406, catheter lumen 1404, the needleless connector 914, etc.) for sensing by sensor 954. As an example, the pressure signal and/or the acoustic signal sensed by sensor 954 may decrease or drop if needle tip 1406 punctures a wall of a blood vessel or urinary tract of the patient. In such an example, a decrease and/or lack in the pressure signal (e.g., a decreased amplitude of a heart rate and/or a drop in blood pressure, etc.) may indicate a lack of a pressure signal associated with an absence of a blood pressure signal, thereby indicating an infiltration event.

In some non-limiting embodiments or aspects, smart device 804 may be programmed and/or configured to compare a relatively slower change or variation in a pressure signal over time (e.g., a relatively slower decrease in an amplitude of a heart rate and/or a drop in blood pressure, etc.) to a threshold level to determine an occlusion event rather than an infiltration event or an extravasation event. For example, an occlusion in a lumen may be at a relatively slow rate over time (e.g., as compared to an infiltration event, an extravasation even, a disconnection event, etc.), which slowly changes in the pressure signal sensed by sensor 954. As an example, smart device 804 may determine an occlusion event and provide an alert and/or automatically flush a lumen associated with the occlusion in response to detection of the occlusion event. In some non-limiting embodiments or aspects, smart device 804 may detect a disconnection event in response to detecting a pressure signal substantially equal to an atmospheric pressure by sensor 954, which indicates that a connection of catheter 1402, e.g., needleless connector 914 is disconnected therefrom and provide an alert to a user to address the connection. In some non-limiting embodiments or aspects, smart device 804 may detect a kink in the catheter lumen 1404 in response to detecting a pressure signal associated with an amplitude of a heart rate that suddenly or immediately drops to zero, as opposed to an occlusion in a lumen that may cause the amplitude of the heart rate to drop a relatively slower rate over time.

In some non-limiting embodiments or aspects, smart device 804 can provide, according to the pressure signal and/or the acoustic signal, a location of the tip of the needle with respect to a blood vessel or a urinary tract of the patient in real-time, thereby providing real-time feedback to a user as a catheter is being installed in a blood vessel or a urinary tract of patient to indicate whether the catheter is properly placed within the blood vessel or the urinary tract or if with one of a potential or existing infiltration of the fluid and a potential or existing extravasation of the fluid. For example, smart device 804 can determine, according to the pressure signal and/or the acoustic signal (e.g., based on a fluid pressure due to fluid entering a catheter path of smart device 804, etc.) a heart rate of a patient, a respiration rate of the patient, a blood pressure of the patient, a penetration force of a needle of the catheter, and/or the like. As an example, smart device 804 can provide, according to the pressure signal and/or the acoustic signal, an indication of entry of the tip of the needle into a blood vessel or a urinary tract of the patient in real-time.

Referring now to FIG. 28, FIG. 28 is a flowchart of a non-limiting embodiment or aspect of a process 2800 for obtaining VAM data. In some non-limiting embodiments or aspects, one or more of the steps of process 2800 are performed (e.g., completely, partially, etc.) by medication source system 802 (e.g., one or more devices of medication source system 802, etc.). In some non-limiting embodiments or aspects, one or more of the steps of process 2800 are performed (e.g., completely, partially, etc.) by another device or a group of devices separate from or including medication source system 802, such as smart device 804 (e.g., one or more devices of a system of smart device 804, etc.), central computing system 808 (e.g., one or more devices of central computing system 808, etc.), and/or terminal/mobile computing system 810 (e.g., one or more devices of terminal/mobile computing system 810, etc.).

As shown in FIG. 28, at step 2802, process 2800 includes obtaining user input associated with a medication source device. For example, medication source system 802 may obtain user input associated with medication source device 906. As an example, medication source system 802 may obtain (e.g., receive, retrieve, determine, etc.) user input received via a user input component (e.g., via pairing input 908, etc.) of medication source device 906. In such an example, medication source system 802 may receive data associated with the user input from medication source device 806.

Referring also to FIG. 22A, in some non-limiting embodiments or aspects, a plurality of medication source devices 906a, 906b, . . . 906n of a medication source system 802 are connected to a plurality of lumens 912a, 912b, . . . 912n, and each medication source 906 device may include a visual indicator 910, communication circuitry (e.g., communication interface 214, etc.), and a paring input 908. In some non-limiting embodiments or aspects, medication source device 906 receives, via pairing input 908 of medication source device 906, user input. For example, visual indicator 910 may emit a predetermined light pattern (e.g., blink rapidly and/or emit a predetermined color to indicate that medication source device 906 is in a pairing mode, etc.) in response to a predetermined user input to pairing input 908 (e.g., in response to a user pressing and holding a button of pairing input 908, etc.) of medication source device 906.

As shown in FIG. 28, at step 2804, process 2800 includes obtaining user input associated with a smart device. For example, medication source system 802 may obtain user input associated with smart device 804. As an example, medication source system 802 may obtain (e.g., receive, retrieve, determine, etc.) user input received via a user input component (e.g., pairing input 956, etc.) of smart device 804. In such an example, medication source system 802 may receive data associated with the user input from smart device 804 that is received at a same time that medication source device 906 is in the pairing mode.

Referring also to FIGS. 22A and 22B, in some non-limiting embodiments or aspects, a plurality of smart devices 804a, 804b, . . . 804n may be connected (e.g., removably connected, etc.) or configured to be connected to the plurality of lumens 912a, 912b, . . . 912n, and each smart device 804 may include a visual indicator 952, communication circuitry (e.g., communication interface 214, etc.), and a paring input 956. In some non-limiting embodiments or aspects, smart device 804 receives, via pairing input 956 of smart device 804, user input. For example, smart device 804 may establish communication with medication source device 906 (e.g., pair and/or activate/initiate a pairing sequence for pairing smart device 804 with medication source device 906, etc.) in response to a predetermined user input to paring input 956 (e.g., in response to a user pressing and holding a button of pairing input 956, etc.) of smart device 804 at a same time that medication source device 906 is in the pairing mode.

As shown in FIG. 28, at step 2806, process 2800 includes establishing communication between a medication source device and a smart device. For example, medication source system 802 may establish communication between medication source device 906 and smart device 804. As an example, medication source system 802 may establish communication (e.g., an NFC communication connection, an RFID communication connection, a Bluetooth® communication connection, and/or the like) between medication source device 906 and smart device 804. In such an example, the communication circuitry of smart device 804 and the communication circuitry of medication source device 906 may establish the communication between (e.g., pair, etc.) smart device 804 and medication source device 906 based on the user input received by pairing input 908 of the medication source device 906 and the user input received by pairing input 956 of smart device 804. For example, medication source device 906 may establish a short range wireless communication connection (e.g., an NFC communication connection, an RFID communication connection, a Bluetooth® communication connection, etc.) with smart device 804. As an example, visual indicator 910 may be configured to emit a predetermined light pattern (e.g., to blink rapidly to indicate that medication source device 906 is in a pairing mode, etc.) in response to a predetermined user input to pairing input 908 (e.g., in response to a user pressing and holding a button of pairing input 908, etc.) of medication source device 906. In such an example, smart device 804 may be configured to establish communication with medication source device 906 (e.g., pair and/or activate a pairing sequence for pairing smart device 804 with medication source device 906, etc.) in response to a predetermined user input to paring input 956 (e.g., in response to a user pressing and holding a button of pairing input 956, etc.) of smart device 804 at a same time that medication source device 906 is in the pairing mode.

As shown in FIG. 28, at step 2808, process 2800 includes controlling visual indicators of a medication source device and a smart device to produce a same type of visual output. For example, medication source system 802 may control visual indicator 910 of medication source device 906 and visual indicator 952 of smart device 804 to produce a same type of visual output. As an example, medication source system 802 may control visual indicator 910 (e.g., a multi-color LED, etc.) of medication source device 906 and visual indicator 952 (e.g., a multi-color LED, etc.) of smart device 804 to produce a same type of visual output (e.g., a same color of light, etc.) based on the communication established between the medication source device and the smart device.

In some non-limiting embodiments or aspects, when smart device 804 is paired with medication source device 906, medication source device 906 may illuminate visual indicator 910 to a color that has not been previously used in medication source system 802 (e.g., that is not associated with another medication source device 906 and another smart device 804 that are paired in medication source system 802, that is different than each other color of light produced by each other smart device 804 of the plurality of smart devices 804a, 804b, . . . 804n and each other medication source device 906 of the plurality of medication source devices 906a, 906b, . . . 906n in medication source system 802, etc.), and smart device 804 may illuminate visual indicator 952 to the same color as visual indicator 910 (e.g., medication source system 802, medication source device 906, smart device 804, etc. may control visual indicator 952 to illuminate to the same color as visual indictor 910). In some non-limiting embodiments or aspects, smart device 804 may illuminate visual indicator 952 to the same color as visual indicator 910 in response to smart device 804 being connected to a lumen and/or during a period of time at which smart device 804 is connected to the lumen. For example, smart device 804 may automatically stop illumination of visual indicator 952 to the same color as visual indicator 910 (e.g., turn off an LED, set the LED to a default color indicating a non-paired smart device 804, etc.) in response to smart device 804 being disconnected from the lumen. As an example, smart device 804 may include a switch connected to visual indicator 952 that is configured to be activated/deactivated in response to a clamp or other connection means being connected/disconnected to a lumen and/or a needleless connector 914 thereof.

In some non-limiting embodiments or aspects, medication source system 802 determines a color of the same color of light for visual indicator 952 of smart device 804 and visual indicator 910 of medication source device 906 to produce based on at least one of the user input received by pairing input 908 of medication source device 906 and the user input received by pairing input 956 of smart device 804. For example, after smart device 804 is paired with medication source device 906, a user may actuate pairing input 908 and/or pairing input 956 to cycle through colors of light available for the pairing to select a desired (and/or available or previously unused) color of light for the pairing.

As shown in FIG. 28, at step 2810, process 2800 includes associating a same type of visual output with a same lumen. For example, medication source system 802 may associate (e.g., automatically associate, etc.) a same type of visual output with a same lumen. As an example, medication source system 802 may associate (e.g., store in connection with, pair, link, illuminate with, etc.) the same type of visual output (e.g., a same color of light, etc.) with a same lumen (e.g., with a same lumen of a plurality of lumens 912a, 912b, . . . 912n, etc.). In such an example, medication source device 906 and smart device 804 may be connected to the same lumen. Accordingly, a user may more easily identify a lumen or line, a location of the lumen or line, a medication that has been or is being delivered via the lumen or line, which infusion pump or mediation source to which the lumen or line is connected, and/or the like.

In some non-limiting embodiments or aspects, medication source system 802 may obtain user input received by a user input component of another medication source device, obtain user input received by a user input component of another smart device, establish a communication between the another medication source device and the another smart device based on the user input received by the user input component of the another medication source device and the user input received by the user input component of the another smart device, control the visual indicator of the another smart device and the visual indicator of the another medication source device to produce another same type of visual output based on the communication established between the another medication source device and the another smart device, wherein the another same type of visual output is different than the same type of visual output, and/or associate the another same type of visual output with another same lumen of the plurality of lumens, wherein the another medication source device is connected to the another same lumen. For example, and referring again to FIG. 22A, medication source device 906a may be paired with smart device 804n and each of medication source device 906a and smart device 804n may output a first color of light (e.g., red light) associated with lumen 912a, medication source device 906b may be paired with smart device 804a and each of medication source device 906b and smart device 804a may output a second color of light (e.g., green light) associated with lumen 912b, medication source device 906n may be paired with smart device 804b and each of medication source device 906n and smart device 804b may output a third color of light (e.g., blue light) associated with lumen 912n, and/or the like.

As shown in FIG. 28, at step 2812, process 2800 includes obtaining VAM data associated with identifying a lumen (e.g., medical device data, etc.). For example, medication source system 802 may identify a lumen and obtain VAM data associated with the identified lumen. As an example, medication source system 802 may identify the same lumen associated with the same type of visual output and obtain VAM data associated with the same lumen associated with the same type of visual output.

In some non-limiting embodiments or aspects, medication source system 802 identifies a lumen by automatically associating and/or providing medical data or VAM data with the same type of visual output associated with the lumen and/or an identifier of the lumen. For example, medical data or VAM data may include at least one of the following: patient data (e.g., an identifier of a particular patient, information and/or data associated with a patient, etc.); medication source data (e.g., an identifier of a particular medication source device 906, etc.); medication data (e.g., an identifier of a type of a medication, a scheduled delivery of a particular medication, a previous delivery of a particular medication, a lumen associated with a medication, etc.); lumen data (e.g., an identifier of a particular lumen, such as the identifier of the same lumen associated with the same type of visual output, etc.); sensor data (e.g., an identifier of a particular sensor 954, information, data, and/or a signal sensed, measured, and/or detected by one or more sensors 954 in one or more smart devices 804, etc.); compliance data (e.g., information or data associated with a scrubbing event in which a needleless connector 914 and/or a lumen is scrubbed with a disinfectant, information or data associated with a flushing event in which a needleless connector 914 and/or a lumen is flushed with a solution, information or data associated with a connection or capping event in which a needleless connector 914 or a lumen is connected to a medical device, etc.); location data (e.g., a location of a patient, a location a previous or scheduled fluid delivery procedure, a location a lumen, a location of a medication source device, etc.); time data (e.g., a time associated with a previous or scheduled fluid delivery procedure, a time of connection of a lumen to medication source device 906, a time of connection of smart device 804 to a lumen, a time of pairing of medication source device 906 and smart device 804, etc.); a location of a tip of a needle of a catheter of a lumen with respect to a blood vessel or urinary tract of the patient; or any combination thereof. As an example, medication source system 802 may obtain medical data from smart device 804, central computing system 808, terminal/mobile computing system 810, one or more databases connected thereto, and/or one or more sensors (e.g., a barcode sensor for scanning a patient identifier, a fluid flow sensor for sensing a flow a fluid, a medication type sensor for sensing a type of a medication, etc.) connected thereto. In such an example, medication source system 802 may identify lumens with information and/or data associated therewith, as well as provide a visual indication of which lumens of a plurality of lumens 912a, 912b, . . . 912n are connected to which medication source devices of a plurality of medication source devices 906a, 906b, . . . 906n, which can enable a user to more easily trace a lumen from a patient to a particular medication source device to which the lumen is connected; connections between lumens and medication source devices to be removed if the patient is moved (e.g., to a new room, to a new floor, to surgery, to the bathroom, etc.) with the same type of visual indicator on a lumen/medication source device pair used to more easily reattach the correct medication source device channel to the correct (e.g., the same as before) lumen; tracking compliance to best practice protocols, for example, by determining if hub scrubbing has occurred and if hub scrubbing occurred effectively (e.g., sufficient pressure, sufficient time scrubbing, etc.) and/or if a device has been flushed, maintained, and/or the like; providing reminders and prescriptive help for protocol adherence, and/or the like.

In some non-limiting embodiments or aspects, medication source system 802 identifies a lumen by determining and providing, based on the medical data, one or more alerts or reminders associated with the lumen and/or the same type of visual output associated with the lumen, such as a reminder to flush the lumen and/or a needleless connector 914 thereof, a reminder to remove or replace a lumen, BD MedMined™ infection prevention guidance (e.g., identification and reporting healthcare-associated infections (HAIs) and using customized alerts and reports to facilitate timely patient intervention, etc.), an alert to use a different lumen for delivery of a particular medication to reduce a chance of a chemical occlusion forming, an alert indicating whether to treat a lumen for thrombus occlusion or chemical occlusion, an alert that an occlusion is detected in a lumen, an alert that a location of a tip of a needle connected to the lumen is associated with one of a potential or existing infiltration of the fluid and a potential or existing extravasation of the fluid, and/or the like.

In some non-limiting embodiments or aspects, medication source system 802 identifies a lumen by controlling a medication source device 906 or another medical device (e.g., an electronic valve, etc.), based on the medical data, to inhibit or prevent delivery of a fluid (e.g., a particular medication, a type of medication, etc.) via the lumen.

Further details regarding non-limiting embodiments or aspects of step 2812 of process 2800 are provided below with regard to FIG. 29.

Referring now to FIG. 29, FIG. 29 is a flowchart of a non-limiting embodiment or aspect of a process 2900 for identifying a lumen. In some non-limiting embodiments or aspects, one or more of the steps of process 2900 are performed (e.g., completely, partially, etc.) by medication source system 802 (e.g., one or more devices of medication source system 802, etc.). In some non-limiting embodiments or aspects, one or more of the steps of process 2900 are performed (e.g., completely, partially, etc.) by another device or a group of devices separate from or including medication source system 802, such as smart device 804 (e.g., one or more devices of a system of smart device 804, etc.), central computing system 808 (e.g., one or more devices of central computing system 808, etc.), and/or terminal/mobile computing system 810 (e.g., one or more devices of terminal/mobile computing system 810, etc.).

As shown in FIG. 29, at step 2902, process 2900 includes obtaining medication data. For example, medication source system 802 may obtain medication data. As an example, medication source system 802 may obtain medication data associated with a first type of medication delivered or scheduled to be delivered via the same lumen to a patient and a second type of medication delivered or scheduled to be delivered via the same lumen to the patient. In such an example, the first type of medication may be different than the second type of medication.

In some non-limiting embodiments or aspects, medication data is associated with at least one of the following: an identifier of a type of a medication, a scheduled delivery of the medication via a particular medication source device, and/or lumen, a previous delivery of the medication via a particular medication source device and/or lumen, an amount of the medication, an identifier of a patient to which the medication is scheduled to be delivered (or delivered), one or more identifiers of one or more different types of medication that are incompatible for delivery via a same lumen with the medication, and/or the like.

As shown in FIG. 29, at step 2904, process 2900 includes determining compatibility of medications. For example, medication source system 802 may determine compatibility of medications. As an example, medication source system 802 may determine, based on the medication data, a compatibility of the second type of medication for delivery via the same lumen as the first type of medication.

In some non-limiting embodiments or aspects, medication source system 802 may use an identifier of the first type of medication and/or an identifier of the second type of medication to access a look-up table that indicates whether the first type of medication and the second type of medication are compatible or incompatible (e.g., compatible or incompatible for delivery via a same lumen, etc.). In some non-limiting embodiments or aspects, the look-up table maybe be stored in and/or associated with the identifier of the first type of medication and/or the identifier of the second type of medication.

In some non-limiting embodiments or aspects, medication source system 802 may obtain medication data associated with a third type of medication delivered or scheduled to be delivered via another same lumen (e.g., different than the same lumen, etc.) to the patient, and determine, based on the medication data, a compatibility of the second type of medication for delivery via the another same lumen as the third type of medication, wherein the indication further indicates whether the second type of medication is compatible for delivery via the another same lumen associated with the another same type of visual output. For example, and referring again to FIGS. 22A and 22B, if medication source device 906 determines that the second type of medication is incompatible for delivery via a first lumen 912a, medication source device 906 may determine a compatibility of the second type of medication for delivery via an alternative lumen, such as a second lumen 912b based a third type of medication delivered or scheduled to be delivered via the second lumen 912b and, if the second type of medication is compatible for delivery via the same lumen as the third type of medication, provide the indication that the second type of medication is compatible for delivery via the second lumen 912b.

As shown in FIG. 29, at step 2906, process 2900 includes obtaining VAM data associated with an indication of compatibility. For example, medication source system 802 may provide an indication of compatibility (e.g., compatibility data, etc.). As an example, medication source system 802 may provide an indication of whether the second type of medication is compatible for delivery via the same lumen associated with the same type of visual output. As another example, medication source system 802 may provide an indication of whether the third type of medication is compatible for delivery via the another same lumen associated with the another same time of visual output.

In some non-limiting embodiment or aspects, medication source system 802 may provide the indication of the compatibility by controlling medication source device 906 to inhibit or prevent delivery of the second medication via the same lumen associated with the same type of visual output. For example, the first type of medication may be delivered to the patient with the same lumen associated with the same type of visual output, and the second type of medication may be scheduled to be delivered via the same lumen to the patient. As an example, and referring again to FIGS. 22A and 22B, medication source system 802 may determine, based on the medical data including the medication data, that a first type of drug is delivered via lumen 912a to the patient and that a second type of drug that is scheduled for delivery or attempting to be delivered via the same lumen 912a is incompatible with the first type of drug (e.g., likely to cause an occlusion, likely to cause an adverse reaction in the patient, etc.). In such an example, medication source system 802 may control medication source device 906a to inhibit or prevent delivery of the second medication via the same lumen 912a (e.g., by stopping a pump, closing a valve, etc.) and/or providing a prompt to the user to use another lumen (e.g., 912b, . . . 912n, etc.) associated with a different type of visual output than the same type of visual output to deliver the second type of medication to the patient.

In some non-limiting embodiments or aspects, the first type of medication and the second type of medication may be delivered to the patient via the same lumen associated with the same type of visual output, and medication source system 802 may provide a prompt to the user to treat the same lumen associated with the same type of visual output for one of a thrombus occlusion and a chemical occlusion. For example, when an occlusion occurs, which may be detected by medication source system 802 as described herein, a user (e.g., a nurse, etc.) may need to determine if the occlusion is thrombotic or chemical due to drug interactions, and medication source system 802 can determine which medications were delivered via which lumens to inform the user of the lumen history and/or provide an indication of a potential cause of the occlusion, which enables a correct decision of whether the lumen should be treated for thrombus or chemical occlusion. In some non-limiting embodiments or aspects, medication source system 802 may control medication source device 906 to automatically perform a flushing operation to deliver a flushing fluid to a lumen connected to the medication source device 906 in response to a determination that an occlusion of the lumen is a chemical occlusion.

Referring now to FIG. 30, FIG. 30 is a flowchart of a non-limiting embodiment or aspect of a process 3000 obtaining VAM data. In some non-limiting embodiments or aspects, one or more of the steps of process 3000 are performed (e.g., completely, partially, etc.) by smart device 804 (e.g., one or more devices of a system of smart device 804, etc.). In some non-limiting embodiments or aspects, one or more of the steps of process 3000 are performed (e.g., completely, partially, etc.) by another device or a group of devices separate from or including smart device 804, such as medication source system 802 (e.g., one or more devices of medication source system 802, etc.), central computing system 808 (e.g., one or more devices of central computing system 808, etc.), and/or terminal/mobile computing system 810 (e.g., one or more devices of terminal/mobile computing system 810, etc.).

As shown in FIG. 30, at step 3002, process 3000 includes obtaining a signal including at least one of a pressure signal and an acoustic signal. For example, smart device 804 may obtain a signal including at least one of a pressure signal and an acoustic signal from at least one sensor connected to a catheter. As an example, smart device 804 may obtain at least one signal including at least one of a pressure signal and an acoustic signal from sensor 954 (e.g., from a pressure sensor, from an acoustic sensor, etc.) connected to catheter 1402. In some non-limiting embodiments or aspects, and referring also to FIG. 27, catheter 1402 includes a needle having tip 1406 for delivering a fluid to a patient.

In some non-limiting embodiments or aspects, sensor 954 measures at least one signal including at least one of a pressure signal and an acoustic signal. For example, sensor 954 may measure the at least one signal including at least one of a pressure signal and an acoustic signal, and smart device 804 (and/or medication source system 802, central computing system 808, and/or terminal/mobile computing system 810) may obtain the at least one signal including at least one of a pressure signal and an acoustic signal from sensor 954. For example, smart device 804 may include communication circuitry (e.g., communication interface 214, etc.) that wirelessly transmits the at least one signal to a remote computing system. As an example, smart device 804 may process the pressure signal and/or the acoustic signal on a microprocessor within a housing of smart device 804 including sensor 954 and the microprocessor, and/or smart device 804 may wirelessly transmit (and/or transmit via wired connection) the pressure signal and/or the acoustic signal to a remote computer that perform digital signal processing on the pressure signal and/or the acoustic signal, to identify and classify events of interest (e.g., infiltration, extravasation, catheter occlusion, etc.).

As shown in FIG. 30, at step 3004, process 3000 includes determining a location of a tip of a needle of a catheter with respect to a blood vessel or a urinary tract of a patient. For example, smart device 804 may determine a location of a tip of a needle with respect to a blood vessel or a urinary tract of a patient. As an example, smart device 804 may determine, based on a variation in the at least one signal over a period of time, a location of tip 1406 of the needle with respect to a blood vessel or a urinary tract of the patient.

In some non-limiting embodiments or aspects, the location of tip 1406 of the needle is determined as one of: within the blood vessel or the urinary tract; within a wall of the blood vessel or a wall of the urinary tract; and outside the blood vessel or the urinary tract and the wall of the blood vessel or the wall of the urinary tract. In some non-limiting embodiments or aspects, smart device 804 and/or one or more components thereof may be connected to or included in (e.g., be integrated with, etc.) a needleless connector 914 at a catheter hub of catheter 1402 located outside the body of the patient. For example, sensor 954 of smart device 804 (e.g., a pressure sensor, an acoustic sensor, etc.) may measure at least one signal including at least one of a pressure signal and an acoustic signal, wherein the catheter includes a needle having a tip for delivering a fluid to a patient.

In some non-limiting embodiments or aspects, smart device 804 determines that the location of tip 1406 of the needle is associated with one of a potential or existing infiltration of the fluid and a potential or existing extravasation of the fluid. For example, sensor 954 (e.g., one or more pressure sensors, one or more acoustic sensors, etc.) may detect temporal variations in a pressure signal and/or an acoustic signal resulting from tip 1406 of the needle of the catheter 1402 being properly inserted in a blood vessel or urinary tract, being located in a wall of the blood vessel or urinary tract, being located outside the blood vessel or urinary tract, and/or the like. As an example, smart device 804 may compare the variation in the at least one signal over the period of time to a threshold variation associated with a heartbeat of the patient. For example, the variations in a pressure signal and/or an acoustic signal may be associated with variations in pressure and/or acoustics in a blood vessel or urinary tract as a result of a heartbeat of the patient. As an example, smart device 804 may compare the variations in the detected pressure signal and/or the detected acoustic signal to variations in a pressure signal and/or an acoustic associated with a heartbeat of the patient to determine if tip 1406 of the needle of catheter 1402 is properly located within the blood vessel (e.g., artery, vein, etc.) of the patient. In such an example, if tip 1406 of the needle of catheter 1402 overshoots the vessel or urinary tract (e.g., punctures a wall of the blood vessel or urinary tract, is not properly within the blood vessel or urinary tract, etc.) the pressure and/or acoustic signature of the at least one signal measured by sensor 954 changes. In some non-limiting embodiments or aspects, infiltration or extravasation of medication into tissues surrounding the blood vessel or urinary tract (rather than into the blood vessel or urinary tract) may result in distinctive pressure or acoustic signals being detected by sensor 954 depending upon the impact of the infiltration or extravasation on surrounding tissues (e.g., if the extravasating medication is a strong vesicant agent such impacts may be severe, etc.).

In some non-limiting embodiments or aspects, smart device 804 determines, based on the variation in the at least one signal over the period of time, at least one of an occlusion of the catheter and a disconnection of the catheter from a needleless connector. For example, smart device 804 may compare the variation in the at least one signal over the period of time to a threshold period of time associated with formation of an occlusion in a catheter. As an example, smart device 804 may compare a relatively slower change or variation in a pressure signal over time (e.g., a relatively slower decrease in an amplitude of a heart rate and/or a drop in blood pressure as compared to an infiltration or extravasation, etc.) to a threshold level to determine an occlusion event rather than an infiltration event or an extravasation event. For example, an occlusion in a lumen may develop at a relatively slow rate over time (e.g., as compared to an infiltration event, an extravasation even, a disconnection event, etc.), which slowly changes the pressure signal sensed may sensor 954. As an example, smart device 804 may determine an occlusion event and provide an alert and/or automatically flush a lumen associated with the occlusion in response to detection of the occlusion event. In some non-limiting embodiments, smart device 804 may detect a disconnection event in response to detecting a pressure signal substantially equal to an atmospheric pressure by sensor 954, which indicates that a connection of catheter 1402, e.g., needleless connector 914 is disconnected therefrom and provide an alert to a user to address the connection.

As shown in FIG. 30, at step 3006, process 3000 includes providing a location of a tip of a needle (e.g., location data, etc.). For example, smart device 804 may provide a location of a tip of a needle. As an example, smart device 804 may provide the location of tip 1406 of the needle with respect to the blood vessel or urinary tract of the patient.

In some non-limiting embodiments or aspects, smart device 804 controls a warning device to issue a warning associated with the one of the potential or existing infiltration of the fluid and the potential or existing extravasation of the fluid. For example, smart device 804 controls visual indicator 952 of smart device 804 to output a color and/or a pattern of light associated with the one of the potential or existing infiltration of the fluid and the potential or existing extravasation of the fluid. As an example, in response to determining an event as infiltration, extravasation, or catheter occlusion, smart device 804 may flash a warning light to a user (e.g., a clinician, a caregiver, a family member, another patient in a homecare or assisted living environment, etc.) and/or transmit a signal to a remote computing system (e.g., medication source system 802, central computing system 808, terminal/mobile computing system 810, etc.) to control (e.g., trigger) output of an audio and/or visual alarm at the remote computing system to alert appropriate individuals of the determined event.

In some non-limiting embodiments or aspects, smart device 804 controls medication source device 906 or a valve (e.g., a valve controlling fluid delivery to/from catheter 902, etc.) to stop (e.g., inhibit, prevent, etc.) delivery of the fluid to the catheter and/or from the catheter. As an example, in response to determining an event as infiltration, extravasation, catheter occlusion, or catheter disconnection smart device 804 may send a signal to an infusion device to immediately stop medication infusion or send a signal to a valve or mechanical clamp to block further medication from infusing into the catheter and/or the patient.

In some non-limiting embodiments or aspects, smart device 804 and/or needleless connector may include communication circuitry (e.g., communication interface 214, etc.) that wirelessly transmits the at least one signal to a remote computing system. As an example, smart device 804 and/or needleless connector 914 may process the pressure signal and/or the acoustic signal on a microprocessor within housing 950 of smart device 804 and/or within housing 1102 of needleless connector 914 including sensor 954 and the microprocessor, and/or smart device 804 and/or needleless connector 914 may wirelessly transmit (and/or transmit via a wired connection) the pressure signal and/or the acoustic signal to a remote computer that performs digital signal processing on the pressure signal and/or the acoustic signal, to identify and classify events of interest (e.g., infiltration, extravasation, catheter occlusion, catheter disconnection, etc.).

In some non-limiting embodiments or aspects, smart device 804 may provide real-time feedback during catheter insertion (e.g., via visual indicator 952, output component 212, medication source system 802, etc.) such that a clinician or other person may be alerted as to whether catheter 1402 is being properly inserted and/or as to whether tip 1406 of the needle of catheter 1402 has pierced or is in the process of piercing a blood vessel or a urinary tract and/or has been accidentally disconnected or occluded.

Referring now to FIG. 31, FIG. 31 is a flowchart of a non-limiting embodiment or aspect of a process 3100 for obtaining VAM data. In some non-limiting embodiments or aspects, one or more of the steps of process 3100 are performed (e.g., completely, partially, etc.) by smart device 804 (e.g., one or more devices of a system of smart device 804, etc.). In some non-limiting embodiments or aspects, one or more of the steps of process 3100 are performed (e.g., completely, partially, etc.) by another device or a group of devices separate from or including smart device, such as medication source system 802 (e.g., one or more devices of medication source system 802, etc.), central computing system 808 (e.g., one or more devices of central computing system 808, etc.), and/or terminal/mobile computing system 810 (e.g., one or more devices of terminal/mobile computing system 810, etc.).

As shown in FIG. 31, at step 3102, process 3100 includes obtaining a signal. For example, smart device 804 may obtain a signal. As an example, smart device 804 may obtain a signal (e.g., a force signal, a signal other than a force signal, such as an optical signal, a flow signal, an acoustic signal, a sound signature, a signal associated with a septum movement, a pressure signal, and/or the like, etc.) measured by a sensor 954 (e.g., a force sensor, an optical sensor, a flow sensor, an acoustic sensor, a pressure sensor, etc.) connected to a needleless connector 914 including a fluid flow path. In such an example, sensor 954 may measure, with a sensor connected to a needleless connector including a fluid flow path, a signal, and smart device 804 (and/or medication source system 802, central computing system 808, terminal/mobile computing system 810, etc.) may obtain the signal from sensor 954.

In some non-limiting embodiments or aspects, a signal obtained by smart device 804 may include a measurement of a value at an instantaneous, static, or single point in time (e.g., a force, a pressure, a sound, a vibration, a reflectance, and/or the like, at a single point in time, etc.). In some non-limiting embodiments or aspects, a signal obtained by smart device 804 may include a dynamic or time-varying signal (e.g., a measurement of a value over a period of time, etc.). For example, a time varying force, pressure, stress, strain, and/or the like may include low frequency signal, such as a signal that changes in sub-audible frequencies (e.g., below 20 Hz, etc.), and/or the like, and/or may include a signal in the acoustic range that travels as sound waves propagating through solids, liquids, and/or air. As described in more detail herein with respect to sensor 954, a time-varying signal may be measured with a force sensor, a seismograph, a pressure sensor, an optical sensor, a microphone, an acoustic sensor for air waves in the audible range, a hydrophone, an acoustic sensor for liquid waves, a pickup or a transducer that captures or senses mechanical vibrations, or any combination thereof.

As shown in FIG. 31, at step 3104, process 3100 includes determining an event associated with a needleless connector based on a signal. For example, smart device 804 may determine an event associated with a needleless connector 914 based on a signal. As an example, smart device 804 may determine, based on the signal (e.g., a force signal, a signal other than a force signal, such as an optical signal, a flow signal, an acoustic signal, a sound signature, a signal associated with a septum movement, a pressure signal, and/or the like, etc.), at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof.

In some non-limiting embodiments or aspects, sensor 954 may include force sensor 1202. In some non-limiting embodiments or aspects, force sensor 1202 includes at least one of: a piezoelectric element, a force sensitive resistive (FSR) sensor, a strain gauge, or any combination thereof. In some non-limiting embodiments or aspects, force sensor 1202 is positioned between an outer surface of inner wall 1210 (e.g., an inner harder plastic wall) of needleless connector 914 defining the fluid flow path of needleless connector 914 and an inner surface of an outer wall 1212 (e.g., a softer, a more flexible, a more pliable, a rubber, etc. wall) of needleless connector 914 surrounding the inner wall 1210 of needleless connector 914. In some non-limiting embodiments or aspects, an area between an outer surface of inner wall 1210 (e.g., an inner harder plastic wall) of needleless connector 914 defining the fluid flow path of needleless connector 914 and an inner surface of an outer wall 1212 (e.g., a softer, a more flexible, more, a more pliable, a rubber, etc. wall) of needleless connector 914 surrounding the inner wall 1210 of needleless connector 914, which may be held by a user during cleaning and/or connection to another medical device, may be filled with a rubber or other pliable type material 1214 including force sensors 1202 as force sensing films within the material 1214 between the inner wall 1210 and the outer wall 1212. In some non-limiting embodiments or aspects, force sensors 1202 may be located between inner wall 1210 and outer wall 1212 below threading on and/or proximal to inlet 1104 of needleless connector 914.

In some non-limiting embodiments or aspects, force sensor 1202 includes a plurality of force sensors 1202 positioned around the fluid flow path of needleless connector 914 between the outer surface of inner wall 1210 of needleless connector 914 defining the fluid flow path of needleless connector 914 and the inner surface of outer wall 1212 of needleless connector 914 surrounding inner wall 1210 of needleless connector 914. For example, inlet 1104 of needleless connector 914 may include septum 1108 including a surface facing in a first direction, and force sensor 1202 may be configured to detect a force in a second direction perpendicular to the surface of the septum facing in the first direction. As an example, the flushing event, which may include a pulsatile flushing event, may be determined based on the force signal indicating periodic forces in the second direction perpendicular to the surface of the septum facing in the first direction.

In some non-limiting embodiments or aspects, sensor 954 includes a pressure sensor, and the pressure sensor is one of: in direct contact with a fluid in the fluid flow path of the needleless connector; located within an inner wall of the needleless connector defining the fluid flow path of the needleless connector, and located within a wall of a lumen connected to the needleless connector. For example, smart device 804 may determine or detect pulsatile flush, a flush, and or a med-administration by the pressure sensor in contact with the fluid path in the needleless connector 914 and/or a lumen thereof.

In some non-limiting embodiments or aspects, the pressure sensor may be configured to sense a pressure transmitted through at least one of a fluid in a catheter and a material of the catheter. For example, and referring again to FIG. 27, needleless connector 914 may be connected to a catheter hub of a catheter 902 including a catheter lumen 1404 and a needle tip 1406 for delivering fluid to a patient at an opposite end of the catheter lumen 1404 from the catheter hub. As an example, the pressure sensor may be connected to the needleless connector 914 to sense the pressure. In such an example, smart device 804 may receive, from the pressure sensor, a signal associated with the sensed pressure and determine, based on the signal, an event associated with the catheter 1402.

In some non-limiting embodiments or aspects, the event associated with the catheter 1402 includes a time at which the needle tip 1406 of the catheter 1402 enters a blood vessel of the patient. For example, smart device 804 may determine the time at which the needle tip 1406 of the catheter 1402 enters the blood vessel based on at least one: of a heart rate, a respiration rate, a blood pressure, a penetration force of the needle tip 1406, or any combination thereof, determined from the signal associated with the sensed pressure.

In some non-limiting embodiments or aspects, the event associated with the catheter 1402 includes a clamping sequence, and smart device 804 may determine the clamping sequence based on one or more changes over time in the signal associated with the sensed pressure. In such an example, smart device 804 may determine, based on the determined clamping sequence and a type of the needleless connector 914 (e.g., a neutral displacement connector, a positive displacement connector, a negative displacement connector, etc.), whether the determined clamping sequence satisfies a clamping protocol associated with the type of the needleless connector 914. For example, different types of needleless connector 914 (e.g., neutral displacement connectors, positive displacement connectors, negative displacement connectors, etc.) may be associated with different clamping protocols recommended to be performed during connection events and/or disconnection events to reduce or prevent backflow into catheter 1402. As an example, not following a claiming protocol associated with the type of needleless connector 914 connected to the catheter 1402 may result in an occlusion in the catheter 1402 or an infection of the patient due to a backflow into the catheter 1402. Accordingly, smart device 804 may reduce or prevent such occlusions and/or infections by monitoring whether a user performs the recommended clamping protocol associated with the particular type of needleless connector 914 connected to catheter 1402.

In some non-limiting embodiments or aspects, the event associated with the catheter 1402 includes an occlusion of the catheter lumen 1404, and smart device 804 may determine the occlusion of the catheter lumen 1404 based on a rate of change in the sensed pressure included in the signal from the pressure sensor. For example, smart device 804 may be programmed and/or configured to compare a relatively slower change or variation in a pressure signal over time (e.g., a relatively slower decrease in an amplitude of a heart rate and/or a drop in blood pressure, etc.) to a threshold level to determine an occlusion event rather than an infiltration event or an extravasation event. For example, an occlusion in a lumen may be at a relatively slow rate over time (e.g., as compared to an infiltration event, an extravasation even, a disconnection event, etc.), which slowly changes in the pressure signal sensed by sensor 954. As an example, smart device 804 may determine an occlusion event and provide an alert and/or automatically flush a lumen associated with the occlusion in response to detection of the occlusion event. In such an example, smart device 804 may detect a kink in the catheter lumen 1404 in response to detecting a pressure signal associated with an amplitude of a heart rate that suddenly or immediately drops to zero, as opposed to an occlusion in a lumen that may cause the amplitude of the heart rate to drop at relatively slower rate over time.

In some non-limiting embodiments or aspects, sensor 954 includes an optical sensor configured to detect a movement of a septum 1108 of needleless connector 914. For example, the optical sensor may be connected to the needleless connector including septum 1108 to detect a movement of the septum 1108. As an example, smart device 804 may receive, from the optical sensor, a signal associated with the movement of the septum and determine, based on the signal, an event associated with the needleless connector 914. For example, the event associated with the needleless connector may include at least one of: a connection event in which the needleless connector 914 is connected to a medical device (e.g., a syringe, a male luer connection, etc.) causing the movement (e.g., a depression, etc.) of septum 1108, a disconnection event in which the needleless connector is disconnected from the medical device cause the movement (e.g., release, etc.) of septum 1108, or any combination thereof. As an example, septum 1108 may include one or more markings, and the optical sensor may be configured to detect a movement of the one or more markings to detect the movement of the septum 1108.

In some non-limiting embodiments or aspects, sensor 954 includes an optical sensor configured to detect at least one of a color signature and a reflectance of a medical device connected to and/or being connected to needleless connector 914, and smart device 804 may determine a type of the medical device based on the at least one of the color signature and the reflectance of the medical device. For example, a color signature and/or the reflectance of the medical device may be indicative of a syringe, an IV bag, an infusion pump, and/or a particular type thereof.

In some non-limiting embodiments or aspects, sensor 954 includes an acoustic sensor. For example, the acoustic sensor may be connected to needleless connector 914 and configured to measure one or more sounds, vibrations, and/or the like (e.g., a sound signature, etc.). As an example, smart device 804 may receive, from the acoustic sensor, a signal including a sound signature, and determine, based on the signal, an event associated with needleless connector 914.

In some non-limiting embodiments or aspects, the event associated with the needleless connector 914 includes (i) a connection event in which the needleless connector 914 is connected to a medical device (e.g., a syringe, a cap, etc.) and/or (ii) an operation of a medical device connected to the needleless connector 914. In such an example, smart device 804 may determine, based on the sound signature (e.g., a sound signature generated from connecting the needleless connector 914 to the medical device, a sound signature generated from operation of the medical device connected to needleless connector 914, one or more ticking sounds, etc.), a type of the medical device connected to the needleless connector 914 from a plurality of types of medical devices and/or a state of the medical device connected to the needleless connector 914. For example, the plurality of types of medical devices may include two or more of the following: a cap, a syringe, a tubing, a medical device connector, or any combination thereof. In some non-limiting embodiments or aspects, smart device 804 may determine, based on the sound signature, a subtype of the determined type of the medical device connected to the needleless connector from a plurality of subtypes of that type of medical device, such as a subtype of a syringe (e.g., a syringe size, a flush syringe, a medication administration syringe, etc.), a subtype of a cap (e.g., a disinfectant cap, etc.), and/or the like. In some non-limiting embodiments or aspects, a state of a medical device includes an unused state or a used state.

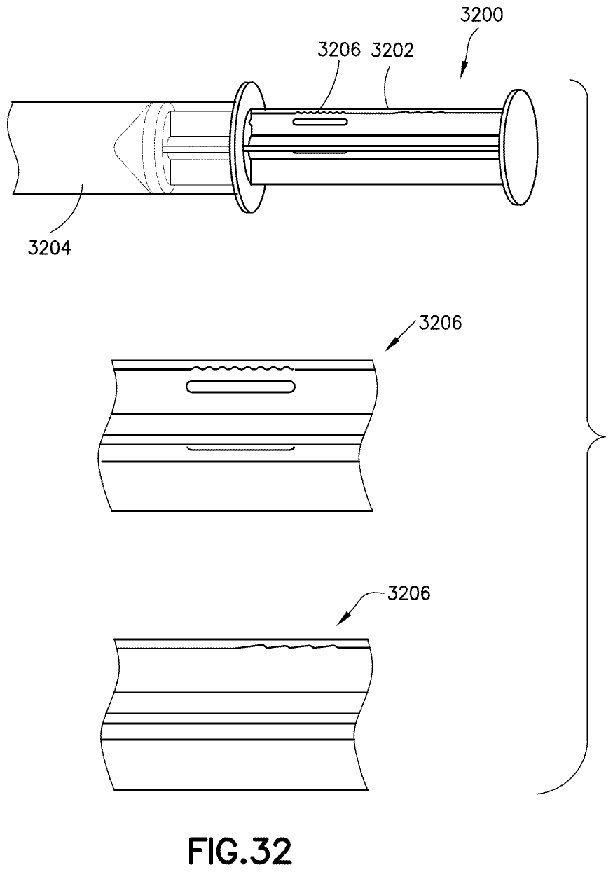

In some non-limiting embodiments or aspects, and referring also to FIG. 32, the medical device includes a syringe 3200. For example, operation of the syringe 3200 may include depression of a plunger 3202 of the syringe 3200 into a barrel 3204 of the syringe 3200, and depression of the plunger 3202 of the syringe 3200 into the barrel 3204 of the syringe 3200 may generate the sound signature (e.g., one or more ticking sounds, etc.). As an example, the plunger 3202 of the syringe 3200 may include one or more extrusions 3206 (e.g., corresponding to the one or more ticking sounds, etc.) that generate the sound signature in combination with the barrel 3204 when the plunger 3202 of the syringe 3200 is depressed into the barrel 3204 of the syringe 3200. In such an example, smart device 804 may differentiate a type and/or state of the syringe 3200 based on the sound signature sensed by the acoustic sensor. For example, the extrusions 3206 may be located or configured to provide an indication of whether a syringe is unused or new (e.g., with plunger 3202 fully extended, which generates a first sound signature in response to depression of plunger 3202 into barrel 3204, etc.) or being re-used (e.g., with plunger 3202 extended half-way, which generates a second sound signature different than the first sound signature (or nor sound signature) in response to further depression of plunger 3202 into barrel 3204, etc.). In some non-limiting embodiments or aspects, a state of the medical device includes a volume of fluid expelled from the syringe when the plunger 3202 of the syringe 3200 is depressed into the barrel 3204 of the syringe 3200. For example, the extrusions 3206 may be located or configured to provide a sound signature associated with an indication of a volume applied by syringe 3200 in response to depression of plunger 3202 within barrel 3204.

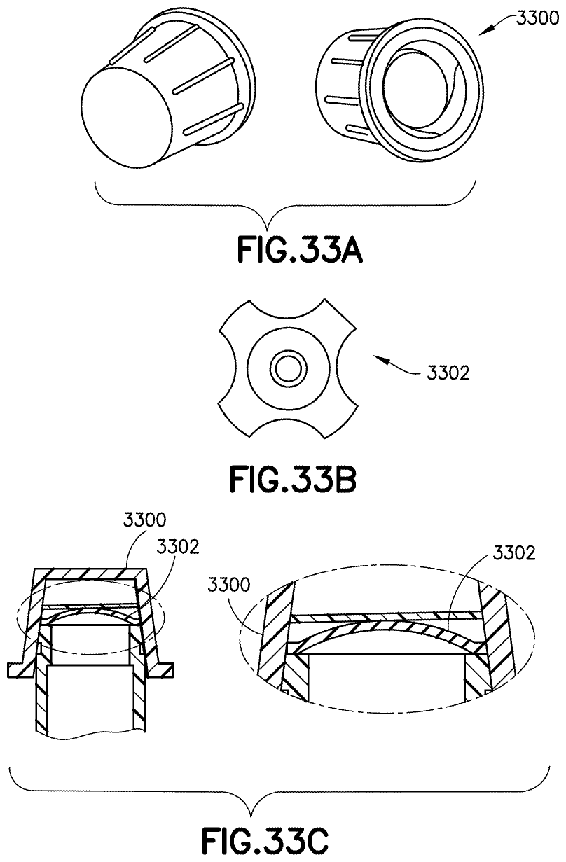

In some non-limiting embodiments or aspects, and referring also to FIGS. 33A-33C, the medical device includes a disinfectant cap 3300. For example, the disinfectant cap 3300 may include a switch 3302 (e.g., a bi-stable metal dome switch, etc.), and the operation of the disinfectant cap 3300 may include a connection of the disinfectant cap 3300 to the needleless connector 914. As an example, connection of the disinfectant cap 3300 to the needleless connector 914 may generate the sound signature when the state of the disinfectant cap 3300 includes the unused state, and, when the state of the disinfectant cap 3300 incudes the used state, the connection of the disinfectant cap 3300 to the needleless connector 914 one of: (i) does not generate the sound signature and (ii) generates another sound signature different than the sound signature generated when the state of the disinfectant cap 3300 includes the unused state. In such an example, a bi-stable metal dome switch incorporated in disinfectant cap 3300 may create a sound signature (e.g., a tick or click sound, etc.) when the disinfectant cap 3300 is attached to a connector, and due to the bi-stable nature, the dome switch stays in position and does not provide a sound signature when the disinfectant cap 3300 is re-used, which may enable detection of disinfectant cap re-use (e.g., if a cap attachment is detected by smart device 804 without the tick or click sound, smart device 804 may determine that the cap is being re-used and/or provide an indication of the re-use, etc.).

In some non-limiting embodiments or aspects, sensor 954 includes an identification sensor configured to detect an identification tag on a medical device connected to or being connected to the needleless connector. For example, the identification sensor may include a magnetometer, and the identification tag may include a magnetic material on and/or integrated with needleless connector 914.

In some non-limiting embodiments or aspects, sensor 954 includes a position sensor configured to detect movement of the needleless connector. For example, a movement of the patient, a fall event of the patient, a movement of a bed of the patient may be determined (e.g., by smart device 804, etc.) based on the detected movement of the needleless connector.

In some non-limiting embodiments or aspects, sensor 954 includes an RGB color sensor configured to detect a color of a fluid in the fluid flow path of the needleless connector. For example, at least one of a blood-draw in the needleless connector and a retention of blood in the needleless connector may be determined (e.g., by smart device 804, etc.) based on the color of the fluid detected in the fluid flow path of the needleless connector.

As shown in FIG. 31, at step 3106, process 3100 includes obtaining VAM data associated with an indication of an event (e.g., event data, etc.). For example, smart device 804 may obtain VAM data associated with an indication of an event. As an example, smart device 804 may provide as the VAM data an indication of the determined event.

In some non-limiting embodiments or aspects, smart device 804 including needleless connector 914 may include visual indicator 952, and visual indicator 952 may be configured to provide a visual indication associated with the at least one of: the scrubbing event in which the needleless connector is scrubbed with the disinfectant, the flushing event in which the needleless connector is flushed with the solution, the connection event in which the needleless connector is connected to the medical device, the disconnection event in which the needleless connector is disconnected from the medical device, or any combination thereof. For example, as shown in an implementation 2600B in FIG. 26B, smart device 804 may provide direct patient-side feedback (e.g., via an LED light to a nurse, etc.) in response to (i) detecting that needleless connector 914 and/or lumen 912 thereof has not been scrubbed for a predetermined period of time and/or before a scheduled use, (ii) detecting that needleless connector 914 and/or lumen 912 thereof has not been scrubbed for a sufficient period of time prior to accessing a catheter line, (iii) detecting that a flush of needleless connector 914 and/or lumen 912 is due, (iv) detecting that a disinfection cap was not attached after a previous access to needleless connector 914 and/or lumen 912, and/or the like. For example, smart device 804 may include needleless connector 914, and needleless connector 914 may be configured to detect at least one of a scrubbing event, a flushing event, a connection or capping event, or any combination thereof. As an example, and needleless connector 914 may be configured to provide information and/or data associated with a detected scrubbing event, a detected flushing event, and/or a detected connection or capping event (e.g., with processor 204, memory 206, storage component 208, input component 210, output component 212, etc.) to store events and report compliance performance for compliance event monitoring.

In some non-limiting embodiments or aspects, smart device 804 may include communication circuitry (e.g., communication interface 214, etc.) that wirelessly transmits the signal (e.g., the force signal, the signal other than the force signal, etc.) and/or an event determined based thereon to a remote computing system. As an example, smart device 804 may process the signal on a microprocessor within a housing of smart device 804 including sensor 954 and the microprocessor, and/or smart device 804 may wirelessly transmit (and/or transmit via wired connection) the signal to a remote computer that performs digital signal processing on the signal, to identify and classify events of interest (e.g., a scrubbing event, a flushing event, a connection event, a disconnection event, a dwell or connection time, etc.).

In some non-limiting embodiments or aspects, a pattern of events including a plurality of the least one of: the scrubbing event in which needleless connector 914 is scrubbed with the disinfectant, the flushing event in which needleless connector 914 is flushed with the solution, connection or capping event in which needleless connector 914 is connected to the medical device, or any combination thereof, may be determined based on the signal (e.g., the force signal, the signal other than a force signal, etc.), and, based on the pattern of events, a medication administration event in which a medication is administered to a patient via needleless connector 914 may be determined.

In some non-limiting embodiments or aspects, smart device 804 may use sensor 954 to detect an identification tag on a medical device connected to or being connected to the needleless connector, movement of the needleless connector, a color of a fluid in the fluid flow path of the needleless connector, or any combination thereof, and provide, with visual indicator 952 visual indication associated with the any information or data sensed and/or measured by sensor 954, such as, a type of the medical device, a medication administration event in which a medication is administered to a patient via the needleless connector, an identification of a medical device, a movement of the patient, a patient fall event, a movement of a bed of the patient, a color of a fluid in the fluid flow path of needleless connector 914, a blood-draw in the needleless connector, a retention of blood in the needleless connector, a scrubbing event in which the needleless connector is scrubbed with a disinfectant, a flushing event in which the needleless connector is flushed with a solution, a connection or capping event in which the needleless connector is connected to a medical device, or any combination thereof.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A system comprising:
at least one processor programmed and/or configured to:
obtain vascular access management (VAM) data associated with a vascular access treatment associated with a patient;
determine an insight associated with the vascular access treatment associated with the patient; and
provide the insight associated with the vascular access treatment.

2. The system of claim 1, wherein the at least one processor is programmed and/or configured to determine the insight associated with the vascular access treatment associated with the patient by:
determining, based on the VAM data, an initial risk prediction for the vascular access treatment associated with the patient, wherein the initial risk prediction includes a probability that the patient experiences at least one complication in response to the vascular access treatment;
determining, based on the VAM data and the initial risk prediction, a recommendation associated with the vascular access treatment associated with the patient, wherein the recommendation includes at least one of a recommended process and a recommend product to be used for the vascular access treatment;
determining, based on the VAM data and the recommendation, an updated risk prediction for the vascular access treatment associated with the patient;
determining, based on the VAM data, the initial risk predication, the recommendation, and the updated risk prediction, a cost prediction associated with the vascular access treatment associated with the patient, wherein the cost prediction includes a predicted savings in terms of a reduced cost of complication from adoption of the at least one of the recommended process and the recommend product.

3. The system of claim 2, wherein the at least one processor provides the insight by providing, to a user device, at least one of the following: the initial risk predication, the recommendation, and the updated risk prediction, the cost prediction, or any combination thereof.

4. The system of claim 1, wherein the at least one processor provides the insight by automatically controlling, based on the insight, at least one medical device to adjust a flow of a fluid to the patient during the vascular access treatment.

5. The system of claim 1, wherein the at least one processor is programmed and/or configured to obtain the VAM data by:
collecting, from a plurality of different data sources, source data;
associating the source data with at least one clinical protocol; and
aggregating the source data associated with the at least one clinical protocol as the VAM data associated with the vascular access treatment associated with the patient.

6. The system of claim 1, wherein the VAM data includes one or more of the following parameters: a patient identifier; a hospital identifier; a patient name; a patient gender; a patient age, a co-morbidity associated with a patient; a medication associated with a patient, a symptom associated with a patient; a reason for admission associated with a patient; an infusion type associated with a patient; an admission date associated with a patient; a readmission indicator associated with a patient; a discharge date associated with a patient; a length of stay associated with a patient; a number of lines used associated with a patient; a type of accessories used associated with a patient; a date of use associated with a medical device; an average dwell time associated with a medical device, an average number of stick attempts associated with a patient, a complication associated with a patient; a department of a hospital; a user or nurse identifier; a user or nurse experience indicator; a question associated with a vascular access treatment; a question identifier associated with a question; an answer associated with a question; a time stamp associated with a usage of a medical device; a device identifier associated with a medical device, a type of a medical device, a device signal associated with a medical device; a number of occlusion cases in a period of time, a number of CRBSI and/or CLABSI cases in a time period; a predicted vascular signal (e.g., CRBSI, phlebitis, etc.); or any combination thereof.

7. The system of claim 1, further comprising:
a plurality of local systems, wherein each local system includes a central computing system, a sensor system including at least one sensor, and a user device; and
a management system configured as a central unit or command center for remotely monitoring line maintenance activities at each local system of the plurality of local systems.

8. The system of claim 1, further comprising:
one or more image capture devices configured to capture, over a period of time, a plurality of images of an environment surrounding the one or more image capture devices; and
wherein the at least one processor is further programmed and/or configured to:
determine, based on the plurality of images, a plurality of locations of a plurality of medical devices within the environment over the period of time and a plurality of types of the plurality of medical devices; and
determine, based on the plurality of locations of the plurality of medical devices within the environment over the period of time and the plurality of types of the plurality of medical devices, at least a portion of the VAM data associated with the vascular access treatment associated with the patient.

9. The system of claim 1, further comprising:
a plurality of identifier elements associated with a plurality of medical devices, wherein the plurality of identifier elements encapsulates a plurality of identifiers associated a plurality of types of the plurality of medical devices; and
one or more image capture devices configured to capture, over a period of time, a plurality of images of an environment surrounding the one or more image capture devices,
wherein the at least one processor is further programmed and/or configured to:
determine, based on the plurality of images, the plurality of identifier elements within the environment over the period of time;
determine, based on the plurality of identifier elements determined in the plurality of images, the plurality of types of the plurality of medical devices and a plurality of locations of the plurality of medical devices within the environment over the period of time; and
determine, based on the plurality of types of the plurality of medical devices and the plurality of locations of the plurality of medical devices within the environment over the period of time, at least a portion of the VAM data associated with the vascular access treatment associated with the patient.

10. The system of claim 9, wherein the plurality of identifier elements includes at least one identifier element including at least one of the following types of identifier elements: a colored pattern, a reflective pattern, a fluorescent pattern, a predetermined shape and color, a LED pattern, a barcode, or any combination thereof.

11. The system of claim 1, further comprising:
one or more image capture devices configured to capture, over a period of time, a plurality of images of an environment surrounding the one or more image capture devices;
wherein the at least one processor is further programmed and/or configured to:
determine, based on the plurality of images, a plurality of locations of a plurality of medical devices within the environment over the period of time and a plurality of types of the plurality of medical devices;
determine, based on the plurality of locations of the plurality of medical devices within the environment over the period of time, a plurality of distances between the plurality of medical devices over the period of time;
determine, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices; and
determine, based on the at least one determined event, at least a portion of the VAM data associated with the vascular access treatment associated with the patient.

12. The system of claim 1, further comprising:
a first identifier element associated with a medical device, wherein the first identifier element encapsulates a first identifier associated with the medical device;
a second identifier element associated with a glove of a caregiver, wherein the second identifier element encapsulates a second identifier associated with the glove of the caregiver;
one or more image capture devices configured to capture, over a period of time, a plurality of images of an environment surrounding the one or more image capture devices; and
wherein the at least one processor is further programmed and/or configured to:
determine, based on the plurality of images, the first identifier element associated with the medical device and the second identifier element associated with the glove of a caregiver;
determine, based on the first identifier element in the plurality of images, the medical device and a location of the medical device within the environment over the period of time;
determine, based on the second identifier element in the plurality of images, the glove of the caregiver and a location of the glove of the caregiver within the environment over the period of time;
determine, based on the location of the medical device within the environment over the period of time and the location of the glove of the caregiver within the environment over the period of time, at least one event associated with the medical device; and
determine, based on the at least one determined event, at least a portion of the VAM data associated with the vascular access treatment associated with the patient.

13. The system of claim 1, further comprising:
one or more image capture devices configured to capture, over a period of time, a plurality of images of an environment surrounding the one or more image capture devices; and
wherein the at least one processor is further programmed and/or configured to:
determine, based on the plurality of images, a location of a plunger of a syringe relative to a barrel of the syringe in the environment over the period of time;
determine, based on the location of the plunger of the syringe relative to the barrel of the syringe over the period of time, at least one fluid delivery from the syringe; and
determine, based on the at least one determined fluid delivery, at least a portion of the VAM data associated with the vascular access treatment associated with the patient.

14. The system of claim 1, further comprising:
a package containing a medical device;
one or more image capture devices configured to capture, over a period of time, a plurality of images of an environment surrounding the one or more image capture devices; and
wherein the at least one processor is further programmed and/or configured to:
determine, based on the plurality of images, a state of the package over the period of time;
determine, based on the state of the package over the period of time, whether the medical device is removed from the package; and
determine, based on a determination that the medical device is removed from the package, at least a portion of the VAM data associated with the vascular access treatment associated with the patient.

15. The system of claim 1, further comprising:
a needleless connector including a fluid flow path; and
a force sensor connected to the needleless connector;
wherein the at least one processor is further programmed and/or configured to:
receive, from the force sensor, a force signal; and
determine, based on the force signal, at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof; and
determine, based on the at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof, at least a portion of the VAM data associated with the vascular access treatment associated with the patient.

16. The system of claim 15, wherein the force sensor is positioned between an outer surface of an inner wall of the needleless connector defining the fluid flow path of the needleless connector and an inner surface of an outer wall of the needleless connector surrounding the inner wall of the needleless connector.

17. The system of claim 15, wherein a first end of the needleless connector includes a septum including a surface facing in a first direction, wherein at least one of the force sensors is configured to detect a force in a second direction perpendicular to the surface of the septum facing in the first direction, and wherein the at least one processor is further programmed and/or configured to:
determine, based on the force signal indicating periodic forces in the second direction perpendicular to the surface of the septum facing in the first direction, the flushing event, wherein the flushing event includes a pulsatile flushing event.

18. The system of claim 1, further comprising:
a needleless connector including a fluid flow path, a force sensor configured to measure a force signal, and a visual indicator,
wherein the at least one processor is further programmed and/or configured to:
receive, from the force sensor, a force signal;
determine, based on the force signal, at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof; and
control the visual indicator to provide a visual indication associated with the at least one of: the scrubbing event in which the needleless connector is scrubbed with the disinfectant, the flushing event in which the needleless connector is flushed with the solution, the connection event in which the needleless connector is connected to the medical device, the disconnection event in which the needleless connector is disconnected from the medical device, or any combination thereof.

19. The system of claim 1, further comprising:
a needleless connector including a fluid flow path;
an acoustic sensor connected to the needleless connector,
wherein the at least one processor is further programmed and/or configured to:
receive, from the acoustic sensor, a signal including a sound signature;
determine, based on the signal, an event associated with the needleless connector; and
determine, based on the determined event associated with the needleless connector, at least a portion of the VAM data associated with the vascular access treatment associated with the patient.

20. The system of claim 1, further comprising:
a needleless connector including a fluid flow path and a septum;
an optical sensor connected to the needleless connector, wherein the optical sensor is configured to detect a movement of the septum,
wherein the at least one processor is further programmed and/or configured to:
receive, from the optical sensor, a signal associated with the movement of the septum;
determine, based on the signal, an event associated with the needleless connector; and
determine, based on the determined event associated with the needleless connector, at least a portion of the VAM data associated with the vascular access treatment associated with the patient.

21. A method comprising:
obtaining, with at least one processor, vascular access management (VAM) data associated with a vascular access treatment associated with a patient;
determining, with the at least one processor, an insight associated with the vascular access treatment associated with the patient; and
providing, with the at least one processor, the insight associated with the vascular access treatment.

22. The method of claim 21, wherein the insight associated with the vascular access treatment associated with the patient is determined by:
determining, based on the VAM data, an initial risk prediction for the vascular access treatment associated with the patient, wherein the initial risk prediction includes a probability that the patient experiences at least one complication in response to the vascular access treatment;
determining, based on the VAM data and the initial risk prediction, a recommendation associated with the vascular access treatment associated with the patient, wherein the recommendation includes at least one of a recommended process and a recommend product to be used for the vascular access treatment;
determining, based on the VAM data and the recommendation, an updated risk prediction for the vascular access treatment associated with the patient;
determining, based on the VAM data, the initial risk predication, the recommendation, and the updated risk prediction, a cost prediction associated with the vascular access treatment associated with the patient, wherein the cost prediction includes a predicted savings in terms of a reduced cost of complication from adoption of the at least one of the recommended process and the recommend product.

23. The method of claim 22, wherein the at least one processor provides the insight by providing, to a user device, at least one of the following: the initial risk predication, the recommendation, and the updated risk prediction, the cost prediction, or any combination thereof.

24. The method of claim 21, wherein the at least one processor provides the insight by automatically controlling, based on the insight, at least one medical device to adjust a flow of a fluid to the patient during the vascular access treatment.

25. The method of claim 21, wherein the at least one processor is obtains the VAM data by:
collecting, from a plurality of different data sources, source data;
associating the source data with at least one clinical protocol; and
aggregating the source data associated with the at least one clinical protocol as the VAM data associated with the vascular access treatment associated with the patient.

26. The method of claim 21, wherein the VAM data includes one or more of the following parameters: a patient identifier; a hospital identifier; a patient name; a patient gender; a patient age, a co-morbidity associated with a patient; a medication associated with a patient, a symptom associated with a patient; a reason for admission associated with a patient; an infusion type associated with a patient; an admission date associated with a patient; a readmission indicator associated with a patient; a discharge date associated with a patient; a length of stay associated with a patient; a number of lines used associated with a patient; a type of accessories used associated with a patient; a date of use associated with a medical device; an average dwell time associated with a medical device, an average number of stick attempts associated with a patient, a complication associated with a patient; a department of a hospital; a user or nurse identifier; a user or nurse experience indicator; a question associated with a vascular access treatment; a question identifier associated with a question; an answer associated with a question; a time stamp associated with a usage of a medical device; a device identifier associated with a medical device, a type of a medical device, a device signal associated with a medical device; a number of occlusion cases in a period of time, a number of CRBSI and/or CLABSI cases in a time period; a predicted vascular signal (e.g., CRBSI, phlebitis, etc.); or any combination thereof.

27. The method of claim 21, further comprising:
remotely monitoring, with a management system configured as a central unit or command center, line maintenance activities at a plurality of local systems, wherein each local system includes a central computing system, a sensor system including at least one sensor, and a user device.

28. The method of claim 21, further comprising:
capturing, with one or more image capture devices, over a period of time, a plurality of images of an environment surrounding the one or more image capture devices; and
determining, with the at least one processor, based on the plurality of images, a plurality of locations of a plurality of medical devices within the environment over the period of time and a plurality of types of the plurality of medical devices; and
determining, with the at least one processor, based on the plurality of locations of the plurality of medical devices within the environment over the period of time and the plurality of types of the plurality of medical devices, at least a portion of the VAM data associated with the vascular access treatment associated with the patient.

29. The method of claim 21, further comprising:
a plurality of identifier elements associated with a plurality of medical devices, wherein the plurality of identifier elements encapsulates a plurality of identifiers associated a plurality of types of the plurality of medical devices; and
capturing, with one or more image capture devices, over a period of time, a plurality of images of an environment surrounding the one or more image capture devices;
determining, with the at least one processor, based on the plurality of images, a plurality of identifier elements within the environment over the period of time, wherein the plurality of identifier elements is associated with a plurality of medical devices, and wherein the plurality of identifier elements encapsulates a plurality of identifiers associated a plurality of types of the plurality of medical devices; and
determining, with the at least one processor, based on the plurality of identifier elements determined in the plurality of images, the plurality of types of the plurality of medical devices and a plurality of locations of the plurality of medical devices within the environment over the period of time.

30. The method of claim 29, wherein the plurality of identifier elements includes at least one identifier element including at least one of the following types of identifier elements: a colored pattern, a reflective pattern, a fluorescent pattern, a predetermined shape and color, a LED pattern, a barcode, or any combination thereof.

31. The method of claim 21, further comprising:
capturing, with one or more image capture devices, over a period of time, a plurality of images of an environment surrounding the one or more image capture devices;
determining, with the at least one processor, based on the plurality of images, a plurality of locations of a plurality of medical devices within the environment over the period of time and a plurality of types of the plurality of medical devices;
determining, with the at least one processor, based on the plurality of locations of the plurality of medical devices within the environment over the period of time, a plurality of distances between the plurality of medical devices over the period of time;
determining, with the at least one processor, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices; and
determining, with the at least one processor, based on the at least one determined event, at least a portion of the VAM data associated with the vascular access treatment associated with the patient.

32. The method of claim 21, further comprising:
capturing, with one or more image capture devices, over a period of time, a plurality of images of an environment surrounding the one or more image capture devices;
determining, with the at least one processor, based on the plurality of images, a first identifier element associated with a medical device and a second identifier element associated with a glove of a caregiver, wherein the first identifier element encapsulates a first identifier associated with the medical device, and wherein the second identifier element encapsulates a second identifier associated with the glove of the caregiver;
determining, with the at least one processor, based on the first identifier element in the plurality of images, the medical device and a location of the medical device within the environment over the period of time;
determining, with the at least one processor, based on the second identifier element in the plurality of images, the glove of the caregiver and a location of the glove of the caregiver within the environment over the period of time;
determining, with the at least one processor, based on the location of the medical device within the environment over the period of time and the location of the glove of the caregiver within the environment over the period of time, at least one event associated with the medical device; and
determining, with the at least one processor, based on the at least one determined event, at least a portion of the VAM data associated with the vascular access treatment associated with the patient.

33. The method of claim 21, further comprising:
capturing, with one or more image capture devices, over a period of time, a plurality of images of an environment surrounding the one or more image capture devices;
determining, with the at least one processor, based on the plurality of images, a location of a plunger of a syringe relative to a barrel of the syringe in the environment over the period of time;
determining, with the at least one processor, based on the location of the plunger of the syringe relative to the barrel of the syringe over the period of time, at least one fluid delivery from the syringe; and
determining, with the at least one processor, based on the at least one determined fluid delivery, at least a portion of the VAM data associated with the vascular access treatment associated with the patient.

34. The method of claim 21, further comprising:
a package containing a medical device;
one or more image capture devices configured to capturing, with one or more image capture devices, over a period of time, a plurality of images of an environment surrounding the one or more image capture devices;
determining, with the at least one processor, based on the plurality of images, a state of a package containing a medical device over the period of time;
determining, with the at least one processor, based on the state of the package over the period of time, whether the medical device is removed from the package; and
determining, with the at least one processor, based on a determination that the medical device is removed from the package, at least a portion of the VAM data associated with the vascular access treatment associated with the patient.

35. The method of claim 21, further comprising:
measuring, with a force sensor connected to a needleless connector including a fluid flow path, a force signal;
receiving, with at least one processor, from the force sensor, the force signal; and
determining, with the at least one processor, based on the force signal, at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof; and
determining, with the at least one processor, based on the at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof, at least a portion of the VAM data associated with the vascular access treatment associated with the patient.

36. The method of claim 35, wherein the force sensor is positioned between an outer surface of an inner wall of the needleless connector defining the fluid flow path of the needleless connector and an inner surface of an outer wall of the needleless connector surrounding the inner wall of the needleless connector.

37. The method of claim 35, wherein a first end of the needleless connector includes a septum including a surface facing in a first direction, wherein at least one of the force sensors is configured to detect a force in a second direction perpendicular to the surface of the septum facing in the first direction, and wherein the method further comprises:
determining, with the at least one processor, based on the force signal indicating periodic forces in the second direction perpendicular to the surface of the septum facing in the first direction, the flushing event, wherein the flushing event includes a pulsatile flushing event.

38. The method of claim 21, further comprising:
measuring, with a force sensor of a needleless connector including a fluid flow path, the force sensor, and a visual indicator, a force signal;
receiving, with the at least one processor, from the force sensor, a force signal;
determining, with the at least one processor, based on the force signal, at least one of: a scrubbing event in which the needleless connector is scrubbed with a disinfectant, a flushing event in which the needleless connector is flushed with a solution, a connection event in which the needleless connector is connected to a medical device, a disconnection event in which the needleless connector is disconnected from the medical device, or any combination thereof; and
controlling, with the at least one processor, the visual indicator to provide a visual indication associated with the at least one of: the scrubbing event in which the needleless connector is scrubbed with the disinfectant, the flushing event in which the needleless connector is flushed with the solution, the connection event in which the needleless connector is connected to the medical device, the disconnection event in which the needleless connector is disconnected from the medical device, or any combination thereof.

39. The method of claim 21, further comprising:
measuring, with an acoustic sensor connected to a needleless connector including a fluid flow path, a signal including a sound signature;
receiving, with the at least one processor, from the acoustic sensor, a signal including a sound signature;
determining, with the at least one processor, based on the signal, an event associated with the needleless connector; and
determining, with the at least one processor, based on the determined event associated with the needleless connector, at least a portion of the VAM data associated with the vascular access treatment associated with the patient.

40. The method of claim 21, further comprising:
measuring, with an optical sensor connected to a needleless connector including a fluid flow path and a septum, a movement of the septum;
receiving, with the at least one processor, from the optical sensor, a signal associated with the movement of the septum;
determining, with the at least one processor, based on the signal, an event associated with the needleless connector; and
determining, with the at least one processor, based on the determined event associated with the needleless connector, at least a portion of the VAM data associated with the vascular access treatment associated with the patient.
